SEQUENCE LISTING (104 sequences)

SEQ ID NO: 1 - protein, 10 aa, synthetic construct
  REGION 1..10: note = Synthetic: 10-MER KRAS G12D-DERIVED PEPTIDE
VVVGADGVGK

SEQ ID NO: 2 - protein, 9 aa, synthetic construct
  REGION 1..9: note = Synthetic: 9-MER KRAS G12D-DERIVED PEPTIDE
VVGADGVGK

SEQ ID NO: 3 - protein, 5 aa, synthetic construct
  REGION 1..5: note = Synthetic: EE209_KRAS_2 / EE209_2 TCR BETA CDR1B
WSHSY

SEQ ID NO: 4 - protein, 6 aa, synthetic construct
  REGION 1..6: note = Synthetic: EE209_2 TCR BETA CDR2B
SAAADI

SEQ ID NO: 5 - skipped

SEQ ID NO: 6 - protein, 14 aa, synthetic construct
  REGION 1..14: note = Synthetic: EE209_KRAS_2 / EE209_2 TCR BETA CDR3B
CASSVGRGGTEAFF

SEQ ID NO: 7 - protein, 6 aa, synthetic construct
  REGION 1..6: note = Synthetic: EE209_KRAS_2 / EE209_2 TCR alpha CDR1a
SSNFYA

SEQ ID NO: 8 - protein, 7 aa, synthetic construct
  REGION 1..7: note = Synthetic: EE209_KRAS_2 / EE209_2 TCR alpha CDR2a
MTLNGDE

SEQ ID NO: 9 - protein, 12 aa, synthetic construct
  REGION 1..12: note = Synthetic: EE209_KRAS_2 / EE209_2 TCR alpha CDR3a
CAFLPGYSTLTF

SEQ ID NO: 10 - protein, 92 aa, synthetic construct
  REGION 1..92: note = Synthetic: EE209_KRAS_2 / EE209_2 TCR alpha variable region
ILNVEQSPQSLHVQEGDSTNFTCSFPSSNFYALHWYRWETAKSPEALFVMTLNGDEKKKGRISATLNTKEGYSYLYIKGSQPEDSATYLCAF

SEQ ID NO: 11 - protein, 95 aa, synthetic construct
  REGION 1..95: note = Synthetic: EE209_KRAS_2 / EE209_2 TCR beta variable region
DAGITQSPRYKITETGRQVTLMCHQTWSHSYMFWYRQDLGHGLRLIYYSAAADITDKGEVPDGYVVSRSKTENFPLTLESATRSQTSVYFCASSV

SEQ ID NO: 12 - protein, 274 aa, synthetic construct
  REGION 1..274: note = Synthetic: EE209_KRAS_2 / EE209_2 TCR alpha
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCAFLPGYSTLTFGKGTMLLVSPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 13 - protein, 311 aa, synthetic construct
  REGION 1..311: note = Synthetic: EE209_KRAS_2 / EE209_2 TCR beta using TRBC2-201 (Bold and underlined portions are differences with SEQ ID NO: 14)
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSVGRGGTEAFFGQGTRLTVVEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 14 - protein, 309 aa, synthetic construct
  REGION 1..309: note = Synthetic: EE209_KRAS_2 / EE209_2 TCR beta using TRBC1-201 (Bold and underlined portions are differences with SEQ ID NOs: 13)
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSVGRGGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 15 - protein, 311 aa, synthetic construct
  REGION 1..311: note = Synthetic: EE209_KRAS_2 / EE209_2 TCR beta using TRBC2-202 (Bold and underlined portions are differences with SEQ ID NO: 13)
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSVGRGGTEAFFGQGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 16 - protein, 5 aa, synthetic construct
  REGION 1..5: note = Synthetic: EE184_KRAS_8A TCR beta CDR1b
LNHDA

SEQ ID NO: 17 - protein, 6 aa, synthetic construct
  REGION 1..6: note = Synthetic: EE184_KRAS_8A TCR beta CDR2b
SQIVND

SEQ ID NO: 18 - protein, 16 aa, synthetic construct
  REGION 1..16: note = Synthetic: EE184_KRAS_8A TCR beta CDR3b
CASSTGAGAFRTEAFF

SEQ ID NO: 19 - protein, 7 aa, synthetic construct
  REGION 1..7: note = Synthetic: EE184_KRAS_8A TCR alpha CDR1a
TSESDYY

SEQ ID NO: 20 - protein, 8 aa, synthetic construct
  REGION 1..8: note = Synthetic: EE184_KRAS_8A TCR alpha CDR2a
QEAYKQQN

SEQ ID NO: 21 - protein, 11 aa, synthetic construct
  REGION 1..11: note = Synthetic: EE184_KRAS_8A TCR alpha CDR3a
CAYKKYNDMRF

SEQ ID NO: 22 - protein, 95 aa, synthetic construct
  REGION 1..95: note = Synthetic: EE184_KRAS_8A TCR alpha variable region
AQTVTQSQPEMSVQEAETVTLSCTYDTSESDYYLFWYKQPPSRQMILVIRQEAYKQQNATENRFSVNFQKAAKSFSLKISDSQLGDAAMYFCAYK

SEQ ID NO: 23 - protein, 95 aa, synthetic construct
  REGION 1..95: note = Synthetic: EE184_KRAS_8A TCR beta variable region
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASST

SEQ ID NO: 24 - protein, 273 aa, synthetic construct
  REGION 1..273: note = Synthetic: EE184_KRAS_8A TCR alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYKKYNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 25 - protein, 311 aa, synthetic construct
  REGION 1..311: note = Synthetic: EE184_KRAS_8A TCR beta chain
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSTGAGAFRTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 26 - protein, 5 aa, synthetic construct
  REGION 1..5: note = Synthetic: PD6_KRASMUT_22 TCR beta CDR1b
SQVTM

SEQ ID NO: 27 - protein, 7 aa, synthetic construct
  REGION 1..7: note = Synthetic: PD6_KRASMUT_22 TCR beta CDR2b
ANQGSEA

SEQ ID NO: 28 - protein, 15 aa, synthetic construct
  REGION 1..15: note = Synthetic: PD6_KRASMUT_22 TCR beta CDR3b
CSVTWAGGWSYGYTF

SEQ ID NO: 29 - protein, 7 aa, synthetic construct
  REGION 1..7: note = Synthetic: PD6_KRASMUT_22 TCR alpha CDR1a
TSESDYY

SEQ ID NO: 30 - protein, 8 aa, synthetic construct
  REGION 1..8: note = Synthetic: PD6_KRASMUT_22 TCR alpha CDR2a
QEAYKQQN

SEQ ID NO: 31 - protein, 14 aa, synthetic construct
  REGION 1..14: note = Synthetic: PD6_KRASMUT_22 TCR alpha CDR3a
CAYYSGGGADGLTF

SEQ ID NO: 32 - protein, 96 aa, synthetic construct
  REGION 1..96: note = Synthetic: PD6_KRASMUT_22 TCR alpha variable region
AQTVTQSQPEMSVQEAETVTLSCTYDTSESDYYLFWYKQPPSRQMILVIRQEAYKQQNATENRFSVNFQKAAKSFSLKISDSQLGDAAMYFCAYYS

SEQ ID NO: 33 - protein, 96 aa, synthetic construct
  REGION 1..96: note = Synthetic: PD6_KRASMUT_22 TCR beta variable region
SAVISQKPSRDICQRGTSLTIQCQVDSQVTMMFWYRQQPGQSLTLIATANQGSEATYESGFVIDKFPISRPNLTFSTLTVSNMSPEDSSIYLCSVT

SEQ ID NO: 34 - protein, 276 aa, synthetic construct
  REGION 1..276: note = Synthetic: PD6_KRASMUT_22 TCR alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 35 - protein, 308 aa, synthetic construct
  REGION 1..308: note = Synthetic: PD6_KRASMUT_22 TCR beta chain
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVTWAGGWSYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 36 - protein, 5 aa, synthetic construct
  REGION 1..5: note = Synthetic: PD6_KRASMUT_9 TCR beta CDR1b
SGHNS

SEQ ID NO: 37 - protein, 6 aa, synthetic construct
  REGION 1..6: note = Synthetic: PD6_KRASMUT_9 TCR beta CDR2b
FNNNVP

SEQ ID NO: 38 - protein, 16 aa, synthetic construct
  REGION 1..16: note = Synthetic: PD6_KRASMUT_9 TCR beta CDR3b
CASSPGPGRHNSPLHF

SEQ ID NO: 39 - protein, 7 aa, synthetic construct
  REGION 1..7: note = Synthetic: PD6_KRASMUT_9 TCR alpha CDR1a
TRDTTYY

SEQ ID NO: 40 - protein, 8 aa, synthetic construct
  REGION 1..8: note = Synthetic: PD6_KRASMUT_9 TCR alpha CDR2a
RNSFDEQN

SEQ ID NO: 41 - protein, 16 aa, synthetic construct
  REGION 1..16: note = Synthetic: PD6_KRASMUT_9 TCR alpha CDR3a
CALSELTTSGTYKYIF

SEQ ID NO: 42 - protein, 96 aa, synthetic construct
  REGION 1..96: note = Synthetic: PD6_KRASMUT_9 TCR alpha variable region
AQKVTQAQTEISVVEKEDVTLDCVYETRDTTYYLFWYKQPPSGELVFLIRRNSFDEQNEISGRYSWNFQKSTSSFNFTITASQVVDSAVYFCALSE

SEQ ID NO: 43 - protein, 96 aa, synthetic construct
  REGION 1..96: note = Synthetic: PD6_KRASMUT_9 TCR beta variable region
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSP

SEQ ID NO: 44 - protein, 278 aa, synthetic construct
  REGION 1..278: note = Synthetic: PD6_KRASMUT_9 TCR alpha chain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSELTTSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 45 - protein, 312 aa, synthetic construct
  REGION 1..312: note = Synthetic: PD6_KRASMUT_9 TCR beta chain
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGPGRHNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 46 - protein, 5 aa, synthetic construct
  REGION 1..5: note = Synthetic: PD22_KRASMUTWT_1 TCR beta CDR1b
MDHEN

SEQ ID NO: 47 - protein, 6 aa, synthetic construct
  REGION 1..6: note = Synthetic: PD22_KRASMUTWT_1 TCR beta CDR2b
SYDVKM

SEQ ID NO: 48 - protein, 16 aa, synthetic construct
  REGION 1..16: note = Synthetic: PD22_KRASMUTWT_1 TCR beta CDR3b
CASSLGPGQHNSPLHF

SEQ ID NO: 49 - protein, 7 aa, synthetic construct
  REGION 1..7: note = Synthetic: PD22_KRASMUTWT_1 TCR alpha CDR1a
TRDTTYY

SEQ ID NO: 50 - protein, 8 aa, synthetic construct
  REGION 1..8: note = Synthetic: PD22_KRASMUTWT_1 TCR alpha CDR2a
RNSFDEQN

SEQ ID NO: 51 - protein, 16 aa, synthetic construct
  REGION 1..16: note = Synthetic: PD22_KRASMUTWT_1 TCR alpha CDR3a
CALSEALAGGSYIPTF

SEQ ID NO: 52 - protein, 90 aa, synthetic construct
  REGION 1..90: note = Synthetic: PD22_KRASMUTWT_1 TCR alpha variable region
AQTEISVVEKEDVTLDCVYETRDTTYYLFWYKQPPSGELVFLIRRNSFDEQNEISGRYSWNFQKSTSSFNFTITASQVVDSAVYFCALSE

SEQ ID NO: 53 - protein, 95 aa, synthetic construct
  REGION 1..95: note = Synthetic: PD22_KRASMUTWT_1 TCR beta variable region
DVKVTQSSRYLVKRTGEKVFLECVQDMDHENMFWYRQDPGLGLRLIYFSYDVKMKEKGDIPEGYSVSREKKERFSLILESASTNQTSMYLCASSL

SEQ ID NO: 54 - protein, 278 aa, synthetic construct
  REGION 1..278: note = Synthetic: PD22_KRASMUTWT_1 TCR alpha chain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEALAGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 55 - protein, 311 aa, synthetic construct
  REGION 1..311: note = Synthetic: PD22_KRASMUTWT_1 TCR beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLGPGQHNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 56 - protein, 5 aa, synthetic construct
  REGION 1..5: note = Synthetic: PD45_KRASMUTWT_4 TCR beta CDR1b
SNHLY

SEQ ID NO: 57 - protein, 6 aa, synthetic construct
  REGION 1..6: note = Synthetic: PD45_KRASMUTWT_4 TCR beta CDR2b
FYNNEI

SEQ ID NO: 58 - protein, 12 aa, synthetic construct
  REGION 1..12: note = Synthetic: PD45_KRASMUTWT_4 TCR beta CDR3b
CASSAGSYEQYF

SEQ ID NO: 59 - protein, 6 aa, synthetic construct
  REGION 1..6: note = Synthetic: PD45_KRASMUTWT_4 TCR alpha CDR1a
DSAIYN

SEQ ID NO: 60 - protein, 7 aa, synthetic construct
  REGION 1..7: note = Synthetic: PD45_KRASMUTWT_4 TCR alpha CDR2a
IQSSQRE

SEQ ID NO: 61 - protein, 16 aa, synthetic construct
  REGION 1..16: note = Synthetic: PD45_KRASMUTWT_4 TCR alpha CDR3a
CAVNSWSGAGSYQLTF

SEQ ID NO: 62 - protein, 92 aa, synthetic construct
  REGION 1..92: note = Synthetic: PD45_KRASMUTWT_4 TCR alpha variable region
KQEVTQIPAALSVPEGENLVLNCSFTDSAIYNLQWFRQDPGKGLTSLLLIQSSQREQTSGRLNASLDKSSGRSTLYIAASQPGDSATYLCAV

SEQ ID NO: 63 - protein, 96 aa, synthetic construct
  REGION 1..96: note = Synthetic: PD45_KRASMUTWT_4 TCR beta variable region
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSA

SEQ ID NO: 64 - protein, 275 aa, synthetic construct
  REGION 1..275: note = Synthetic: PD45_KRASMUTWT_4 TCR alpha chain
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVNSWSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 65 - protein, 310 aa, synthetic construct
  REGION 1..310: note = Synthetic: PD45_KRASMUTWT_4 TCR beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSAGSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 66 - protein, 5 aa, synthetic construct
  REGION 1..5: note = Synthetic: EE208_KRAS_1 TCR beta CDR1b
MGHDK

SEQ ID NO: 67 - protein, 6 aa, synthetic construct
  REGION 1..6: note = Synthetic: EE208_KRAS_1 TCR beta CDR2b
SYGVNS

SEQ ID NO: 68 - protein, 13 aa, synthetic construct
  REGION 1..13: note = Synthetic: EE208_KRAS_1 TCR beta CDR3b
CASSESLYNEQFF

SEQ ID NO: 69 - protein, 6 aa, synthetic construct
  REGION 1..6: note = Synthetic: EE208_KRAS_1 TCR alpha CDR1a
ATGYPS

SEQ ID NO: 70 - protein, 7 aa, synthetic construct
  REGION 1..7: note = Synthetic: EE208_KRAS_1 TCR alpha CDR2a
ATKADDK

SEQ ID NO: 71 - protein, 14 aa, synthetic construct
  REGION 1..14: note = Synthetic: EE208_KRAS_1 TCR alpha CDR3a
CALSDGAGGFKTIF

SEQ ID NO: 72 - protein, 93 aa, synthetic construct
  REGION 1..93: note = Synthetic: EE208_KRAS_1 TCR alpha variable region
GNSVTQMEGPVTLSEEAFLTINCTYTATGYPSLFWYVQYPGEGLQLLLKATKADDKGSNKGFEATYRKETTSFHLEKGSVQVSDSAVYFCALS

SEQ ID NO: 73 - protein, 95 aa, synthetic construct
  REGION 1..95: note = Synthetic: EE208_KRAS_1 TCR beta variable region
EADIYQTPRYLVIGTGKKITLECSQTMGHDKMYWYQQDPGMELHLIHYSYGVNSTEKGDLSSESTVSRIRTEHFPLTLESARPSHTSQYLCASSE

SEQ ID NO: 74 - protein, 273 aa, synthetic construct
  REGION 1..273: note = Synthetic: EE208_KRAS_1 TCR alpha chain
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSDGAGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 75 - protein, 310 aa, synthetic construct
  REGION 1..310: note = Synthetic: EE208_KRAS_1 TCR beta chain
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSESLYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 76 - protein, 5 aa, synthetic construct
  REGION 1..5: note = Synthetic: OX92_KRAS_36 TCR beta CDR1b
SGHVS

SEQ ID NO: 77 - protein, 6 aa, synthetic construct
  REGION 1..6: note = Synthetic: OX92_KRAS_36 TCR beta CDR2b
FQNEAQ

SEQ ID NO: 78 - protein, 13 aa, synthetic construct
  REGION 1..13: note = Synthetic: OX92_KRAS_36 TCR beta CDR3b
CASSLTGAGEQYF

SEQ ID NO: 79 - protein, 6 aa, synthetic construct
  REGION 1..6: note = Synthetic: OX92_KRAS_36 TCR alpha CDR1a
DSSSTY

SEQ ID NO: 80 - protein, 7 aa, synthetic construct
  REGION 1..7: note = Synthetic: OX92_KRAS_36 TCR alpha CDR2a
IFSNMDM

SEQ ID NO: 81 - protein, 14 aa, synthetic construct
  REGION 1..14: note = Synthetic: OX92_KRAS_36 TCR alpha CDR3a
CAERDPGNNRKLIW

SEQ ID NO: 82 - protein, 91 aa, synthetic construct
  REGION 1..91: note = Synthetic: OX92_KRAS_36 TCR alpha variable region
GEDVEQSLFLSVREGDSSVINCTYTDSSSTYLYWYKQEPGAGLQLLTYIFSNMDMKQDQRLTVLLNKKDKHLSLRIADTQTGDSAIYFCAE

SEQ ID NO: 83 - protein, 96 aa, synthetic construct
  REGION 1..96: note = Synthetic: OX92_KRAS_36 TCR beta variable region
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASSL

SEQ ID NO: 84 - protein, 274 aa, synthetic construct
  REGION 1..274: note = Synthetic: OX92_KRAS_36 TCR alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAERDPGNNRKLIWGLGTSLAVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 85 - protein, 311 aa, synthetic construct
  REGION 1..311: note = Synthetic: OX92_KRAS_36 TCR beta chain
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLTGAGEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 86 - protein, 5 aa, synthetic construct
  REGION 1..5: note = Synthetic: EE231_KRAS_52 TCR beta CDR1b
MDHEN

SEQ ID NO: 87 - protein, 6 aa, synthetic construct
  REGION 1..6: note = Synthetic: EE231_KRAS_52 TCR beta CDR2b
SYDVKM

SEQ ID NO: 88 - protein, 16 aa, synthetic construct
  REGION 1..16: note = Synthetic: EE231_KRAS_52 TCR beta CDR3b
CASSFGPGQHNSPLHF

SEQ ID NO: 89 - protein, 6 aa, synthetic construct
  REGION 1..6: note = Synthetic: EE231_KRAS_52 TCR alpha CDR1a
RDTTYY

SEQ ID NO: 90 - protein, 8 aa, synthetic construct
  REGION 1..8: note = Synthetic: EE231_KRAS_52 TCR alpha CDR2a
RNSFDEQN

SEQ ID NO: 91 - protein, 16 aa, synthetic construct
  REGION 1..16: note = Synthetic: EE231_KRAS_52 TCR alpha CDR3a
CALSEARTGGSYIPTF

SEQ ID NO: 92 - protein, 96 aa, synthetic construct; identical to SEQ ID NO: 42
  REGION 1..96: note = Synthetic: EE231_KRAS_52 TCR alpha variable region

SEQ ID NO: 93 - protein, 95 aa, synthetic construct
  REGION 1..95: note = Synthetic: EE231_KRAS_52 TCR beta variable region
DVKVTQSSRYLVKRTGEKVFLECVQDMDHENMFWYRQDPGLGLRLIYFSYDVKMKEKGDIPEGYSVSREKKERFSLILESASTNQTSMYLCASSF

SEQ ID NO: 94 - protein, 278 aa, synthetic construct
  REGION 1..278: note = Synthetic: EE231_KRAS_52 TCR alpha chain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEARTGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 95 - protein, 311 aa, synthetic construct
  REGION 1..311: note = Synthetic: EE231_KRAS_52 TCR beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFGPGQHNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 96 - DNA, 20 bp, synthetic construct
  misc_feature 1..20: note = Synthetic: sgTRAC
  source 1..20: mol_type = other DNA
agagtctctcagctggtaca

SEQ ID NO: 97 - DNA, 20 bp, synthetic construct
  misc_feature 1..20: note = Synthetic: sgTRBC
  source 1..20: mol_type = other DNA
ggagaatgacgagtggaccc

SEQ ID NO: 98 - DNA, 20 bp, synthetic construct
  misc_feature 1..20: note = Synthetic: sgRAB11A
  source 1..20: mol_type = other DNA
ggtagtcgtactcgtcgtcg

SEQ ID NO: 99 - DNA, 20 bp, synthetic construct
  misc_feature 1..20: note = Synthetic: sgCD4
  source 1..20: mol_type = other DNA
ggcaggtcttcttctcactg

SEQ ID NO: 100 - DNA, 20 bp, synthetic construct
  misc_feature 1..20: note = Synthetic: sgTNFRSF9
  source 1..20: mol_type = other DNA
gttgaggaccagcaacagag

SEQ ID NO: 101 - DNA, 20 bp, synthetic construct
  misc_feature 1..20: note = Synthetic: sgAAVS1
  source 1..20: mol_type = other DNA
gggaccaccttatattccca

SEQ ID NO: 102 - protein, 10 aa, synthetic construct
  REGION 1..10: note = Synthetic: wild type KRAS peptide
VVVGAGGVGK

SEQ ID NO: 103 - protein, 10 aa, synthetic construct
  REGION 1..10: note = Synthetic: KRAS-p.G12V peptide
VVVGAVGVGK

SEQ ID NO: 104 - protein, 9 aa, synthetic construct
  REGION 1..9: note = Synthetic: 9-mer wildtype KRAS peptide
VVGAGGVGK

(19) United States
(12) Patent Application Publication
(10) Pub. No.: US 2026/0232734 A1
(43) Pub. Date: Aug. 13, 2026

(54) Therapeutic T-Cell Receptors Targeting KRAS G12D

(71) Applicant: Adaptive Biotechnologies Corporation, Seattle, WA (US)

(72) Inventors: Mark Klinger, Seattle, WA (US); Soyoung Oh, South San Francisco, CA (US); Sascha Rutz, South San Francisco, CA (US); Rena Whei-Ming Yang, South San Francisco, CA (US); Erik Yusko, Seattle, WA (US); Peter Ebert, Seattle, WA (US); Kate Halliwell Senger, South San Francisco, CA (US)

(21) Appl. No.: 19/159,272

(22) PCT Filed: Feb. 23, 2024
(86) PCT No.: PCT/US2024/016986

§ 371 Date: Aug. 22, 2025

Related U.S. Application Data
(60) Provisional application No. 63/487,081, filed on Feb. 27, 2023.

(51) Int. Cl. A61K 35/17; A61K 40/11; A61K 40/24; A61K 40/32; A61K 40/42; A61P 35/00; C07K 16/28

(52) U.S. Cl. CPC A61K 35/17; A61K 40/11; A61K 40/24; A61K 40/32; A61K 40/42; A61P 35/00; C07K 16/2827; A61K 2239/28; A61K 2239/54

(57) ABSTRACT

Disclosed herein are therapeutic T cell receptors (TCRs) that bind to a KRAS G12D neoantigen, engineered cells expressing such TCRs, pharmaceutical compositions comprising such cells, and methods of treatment using such cells or pharmaceutical compositions.

### CROSS-REFERENCE TO RELATED APPLICATIONS

This application claims the benefit of priority of U.S. Provisional Application No. 63/487,081, filed Feb. 27, 2023, which is incorporated by reference herein in its entirety for any purpose.

### REFERENCE TO ELECTRONIC SEQUENCE LISTING

The application contains a Sequence Listing which has been submitted electronically in XML format. Said .XML copy, created on Feb. 20, 2024, is named “01146-0115-00PCT.xml” and is 139,083 bytes in size. The sequence listing contained in this .XML file is part of the specification and is hereby incorporated by reference herein in its entirety.

### DESCRIPTION

### Field

Compositions and methods for treating cancer using novel T cell receptors (TCRs), administered in some instances alone and in some instances in a combination therapy with an anti-PD-L1 antibody.

### Background of the Invention

It is believed that several important diseases arise from aberrant T cell function: For example, cancers are thought to arise from a failure of immune surveillance, that is, the T cell function of detecting and destroying clones of transformed cells before they grow into tumors; and autoimmune diseases are thought to arise from an over active or aberrant response of T cells to self-antigens (Finn, et al, “Immuno-oncology: understanding the function and dysfunction of the immune system in cancer,” Annals of Oncology, vol. 23, supp. 8: viii6-viii9 (2012)).

T cell receptor (TCR) T cell therapies introduce cytotoxic T cells engineered to express pre-selected potent TCRs targeting unique tumor antigens in patients. Engineered T cells may overcome limitations from therapeutic vaccines or checkpoint inhibitors that rely on the activity of endogenous T cells to mediate anti-tumor responses, with clinical benefits limited to a subset of patients thus far.

The antigen-specificity of T cells is mediated by the heterodimeric TCR, which consists of α and β membrane-bound subunits that couple with CD3 complexes, leading to intracellular signal transduction when presented with major histocompatibility complex (MHC)-bound antigens. Progress in identifying and sequencing TCRs with desired specificity as well as advances in gene delivery have made it possible to engineer large populations of T cells to express a recombinant tumor antigen-specific TCR. Most current approaches in the clinic use readily accessible autologous T cells derived from peripheral blood and transduce them with retro- or lentiviruses encoding the α and β chains of the desired TCR without extensive in vitro expansion, likely improving the fitness of the cell product.

KRAS is the most frequently mutated oncogene with mutations found in approximately 14% of all cancers (Lee et al, “Comprehensive pan-cancer genomic landscape of KRAS altered cancers and real-world outcomes in solid tumors,” NPJ Precis Oncol 2022; 6:91). A mutation at amino acid position glycine 12 (G12) is commonly found in solid tumors and associated with tumorigenesis and aggressive tumor growth. (Der et al, “Transforming genes of human bladder and lung carcinoma cell lines are homologous to the ras genes of Harvey and Kirsten sarcoma viruses,” PNAS Vol. 79(11) 3637-3640 (1982); Parada et al, “Human EJ bladder carcinoma oncogene is homologue of Harvey sarcoma virus ras gene,” Nature, 197, 474 (1982); Santos et al, “T24 human bladder carcinoma oncogene is an activated form of the normal human homologue of BALB, and Harvey-MSV transforming genes,” Nature, 298, 343 (1982); Taparowsky et al, “Activation of the T24 bladder carcinoma transforming gene is linked to a single amino acid change,” Nature, 300, 762 (1982); Capon et al, “Activation of Ki-ras2 gene in human colon and lung carcinomas by two different point mutations,” Nature, 304, 507-513 (1983).) Because the peptides derived from these cancer-specific alterations can be presented by HLA receptors on the surface of cancer cells, they are attractive as mutant cancer neoantigen targets for T cell therapies with applicability in a number of indications. Specifically, oncogenic KRAS mutations that result in the change from G12 to aspartic acid (G12D) are prevalent in pancreatic ductal adenocarcinoma (PDAC) (40% of tumors), colorectal cancer (CRC) (15% of tumors), non-small cell lung cancer (NSCLC) (5% of tumors) as well as in other tumor types (Lee et al. 2022).

Pancreatic cancer is the seventh leading cause of cancer deaths worldwide and the fourth leading cause of cancer deaths in the United States (Dalmartello et al, “European cancer mortality predictions for the year 2022 with focus on ovarian cancer,” Ann Oncol. Mar; 33(3): 330-339 (2022); Siegel et al. 2022; American Cancer Society 2022). Pancreatic cancer is predicted to become the second leading cause of cancer deaths by 2030 (Rahib et al, “Projecting cancer incidence and deaths to 2030: the unexpected burden of thyroid, liver, and pancreas cancers in the United States,” Cancer Res. 2014 Jun. 1; 74(11):2913-21 (2014), Erratum in: Cancer Res. July 15; 74(14): 4006 (2014); Siegel et al, “Cancer Statistics, 2022,” CA vol. 72:1 (2022).) Pancreatic ductal adenocarcinoma (PDAC), which develops in the exocrine tissue of the pancreas, is responsible for approximately 90% of pancreatic cancer cases. Overall, PDAC has a 5-year survival rate under 10% (Haeberle et al, “Pathology of pancreatic cancer,” Transl Gastroenterol Hepatol. 2019 Jun. 27; 4:5 (2019).) For patients who have locally advanced and unresectable disease, radiochemotherapy is often utilized, resulting in a median overall survival (OS) of 9-13 months, but rarely offering long-term survival (Czito et al, “Current perspectives on locally advanced pancreatic cancer,” Oncology 2000; 14:1535-45). For patients with metastatic disease at initial diagnosis, prognosis is worse, with a median OS of 8.5-11.1 months with first-line treatment of FOLFIRINOX (leucovorin, 5-fluorouracil [5-FU], irinotecan, and oxaliplatin) or gemcitabine and nab-paclitaxel (Conroy et al, “FOLFIRINOX versus gemcitabine for metastatic pancreatic cancer,” N Engl J Med 2011; 364:1817-25; Von Hoff et al, “Increased survival in pancreatic cancer with nab-paclitaxel plus gemcitabine,” N Engl J Med 2013; 369:1691). Immunotherapies (pembrolizumab) have shown activity in PDAC but only benefit the less than 2% of PDAC patients with high microsatellite instability (MSI-H) or mismatch repair deficiency (dMMR) tumors. Therefore, there is a high unmet need for improved therapeutic options for patients with PDAC.

Colorectal cancer is the third leading cause of death in the United States (American Cancer Society, Key statistics for Colorectal Cancer, Jan. 13, 2023). There remains a high unmet need for this patient population for more effective treatment options with better safety profiles. In the majority of patients with mCRC, systemic cytotoxic chemotherapy is the mainstay of treatment with median overall survival (OS) of approximately 30 months in the first-line treatment setting. Second-line therapy is usually directed by using an agent not used in the first-line treatment, but treatment remains palliative rather than curative. Once standard chemotherapy regimens have been exhausted, patient survival is usually less than 6 months. Immunotherapies have demonstrated durable response but only in a small subset of patients (3%-5%) with microsatellite instability-high (MSI-H) or mismatch-repair deficiency (dMMR) (Lee et al, “Mismatch repair deficiency and response to immune-checkpoint blockade,” Oncologist 21:1200-11 (2016)). To date, checkpoint inhibitors offer little to no clinical benefit for the approximately 95% of patients with mCRC that are microsatellite stable (MSS) or microsatellite instability-low (MSI-L). Therefore, there remains a high unmet medical need for the development of new effective therapies for the vast majority of patients with MSS/MSI-L mCRC. KRAS mutations are associated with resistance to and lack of patient benefit from anti EGFR monoclonal antibody therapies (Lièvre et al, “KRAS mutation status is predictive of response to cetuximab therapy in colorectal cancer,” Cancer Res 2006; 66:3992-5; Karapetis et al., “K-ras mutations and benefit from cetuximab in advanced colorectal cancer,” N Engl J Med 2008; 359:1757-65; Van Cutsem, et al, “Cetuximab and chemotherapy as initial treatment for metastatic colorectal cancer,” N Engl J Med 2009; 360:1408-17; Misale, “Emergence of KRAS mutations and acquired resistance to anti-EGFR therapy in colorectal cancer,” Nature 2012; 486:532-36). Therefore, patients whose cancers harbor KRAS mutations, such as KRAS G12D, are not eligible for treatment with cetuximab or panitumumab (NCCN 2020b) and have even more limited treatment options.

Lung cancer is the leading cause of cancer deaths worldwide. NSCLC is one of the two major types of lung cancer, accounting for approximately 85% of all lung cancer cases (Molina et al, “Non-small cell lung cancer: epidemiology, risk factors, treatment, and survivorship,” Mayo Clin Proc 83:584-94 (2008)) with the majority of patients diagnosed with metastatic disease (Aupérin et al, “Meta-analysis of concomitant versus sequential radiochemotherapy in locally advanced non-small-cell lung cancer,” J Clin Oncol 28:2181090 (2010)). Although a minority of patients achieve long-term disease control with earlier lines of treatment in general very few effective treatments exist beyond the second line for patients with advanced stage or metastatic NSCLC. Patients with KRAS G12C mutations can be treated with newly approved small molecule inhibitors (sotorasib, adagrasib) in the second line that have shown clinical benefit in trials (Janne et al, “Adagrasib in Non-Small-Cell Lung Cancer Harboring a KRASG12C Mutation,” N Engl J Med 387:120-131 (2022); Skoulidis et al, “Sotorasib for lung cancers with KRAS p.G12C mutation,” N Engl J Med 384:2371-81 (2021)). Unfortunately, no targeted agents are currently approved for patients with KRAS G12D mutations.

Patients with advanced-stage KRAS G12D-positive cancers may derive limited benefit from select chemotherapies and targeted therapies, thus restricting available effective treatment options (Román et al, “KRAS oncogene in non-small cell lung cancer: clinical perspectives on the treatment of an old target,” Mol Cancer 17:33 (2018); Wang et al, “KRAS Mutant Allele Fraction in Circulating Cell-Free DNA Correlates With Clinical Stage in Pancreatic Cancer Patients,” Front. Oncol. 9:1295 (2019)). Despite KRAS G12D having a well-established role in cancer, approved therapies targeting tumors with the mutation remain unavailable. A T cell therapy that specifically recognizes the KRAS G12D neoantigen and can be used to treat KRAS G12D-positive cancers thus represents a novel approach that can potentially benefit patients with a high unmet need.

### SUMMARY OF THE INVENTION

The present disclosure provides recombinant T cell receptors (TCRs) that specifically bind to a Kirsten rat sarcoma viral oncogene homolog (KRAS) G12D neoantigen, engineered cells expressing such TCRs, pharmaceutical compositions comprising such engineered cells, methods for treating cancer using the same. In some embodiments, the present disclosure provides one or more of the CDRs, variable regions, or alpha (α) chain and the beta (β) chain sequences of antigen-specific TCRs (e.g., KRAS G12D neoantigen specific TCR alpha and/or beta chains), and engineered cells comprising such sequences. In some embodiments, the present disclosure provides TCRs and T cells exogenously expressing TCRs specific for KRAS G12D neoantigen that are useful in therapeutic and/or diagnostic methods for KRAS G12D-expressing cancers.

Embodiment 1. A recombinant T cell receptor (TCR) that binds to a Kirsten rat sarcoma viral oncogene homolog (KRAS) G12D neoantigen comprising:
a TCR-alpha chain comprising a TCR-alpha variable region; and
a TCR-beta chain comprising a TCR-beta variable region;
wherein the TCR-alpha variable region comprises the following:
   a CDR1 comprising the amino acid sequence SEQ ID NO: 7,
   a CDR2 comprising the amino acid sequence SEQ ID NO: 8, and
   a CDR3 comprising amino the acid sequence SEQ ID NO: 9; and
wherein the TCR-beta variable region comprises the following:
   a CDR1 comprising the amino acid sequence SEQ ID NO: 3,
   a CDR2 comprising the amino acid sequence SEQ ID NO: 4, and
   a CDR3 comprising the amino acid sequence SEQ ID NO: 6.

Embodiment 2. The recombinant TCR of embodiment 1, wherein the TCR-alpha chain variable region comprises at least 95%, at least 96%, at least 97%, at least 98%, at least 99%, or 100% sequence identity to the amino acid sequence set forth in SEQ ID NO: 10 and the TCR-beta chain variable region comprises at least 95%, 96%, 97%, 98%, 99%, or 100% sequence identity to the amino acid sequence set forth in SEQ ID NO: 11.

Embodiment 3. The recombinant TCR of embodiment 1 or 2, wherein the TCR-alpha chain comprises at least 95%, at least 96%, at least 97%, at least 98%, at least 99%, or 100% sequence identity to the amino acid sequence set forth in SEQ ID NO: 12.

Embodiment 4. The recombinant TCR of any one of embodiments 1-3, wherein the TCR-beta chain comprises at least 95%, at least 96%, at least 97%, at least 98%, at least 99%, or 100% sequence identity to the amino acid sequence set forth in SEQ ID NO: 13, SEQ ID NO: 14, or 15.

Embodiment 5. The recombinant TCR of any one of embodiments 1-4, wherein the TCR binds the KRAS G12D neoantigen in a subject who is HLA-A*11:01 positive.

Embodiment 6. The recombinant TCR of any one of embodiments 1-5, wherein the TCR is HLA 11:01 restricted.

Embodiment 7. The recombinant TCR of any one of embodiments 1-6, wherein the TCR activates a T cell upon binding to the KRAS G12D neoantigen.

Embodiment 8. The recombinant TCR of any one of embodiments 1-7, wherein the neoantigen comprises SEQ ID NO: 1 or SEQ ID NO: 2.

Embodiment 9. An engineered cell that expresses the recombinant TCR of any one of embodiments 1-8.

Embodiment 10. The engineered cell of embodiment 9 wherein the cell is an iPSC-derived T cell, a patient-derived autologous T cell, a donor-derived T cell, or an iPSC cell, optionally wherein the patient-derived autologous T cell, donor-derived T cell, or iPSC-derived T cell is a CD8+ T cell.

Embodiment 11. The engineered cell of embodiment 9 or 10 wherein the cell is an iPSC-derived T cell.

Embodiment 12. The engineered cell of embodiment 9 or 10 wherein the cell is a patient-derived autologous T cell.

Embodiment 13. The engineered cell of any one of embodiments 9-12, wherein the T cell, which is optionally an iPSC-derived T cell, or iPSC cell comprises at least one nucleic acid sequence comprising at least one heterologous gene inserted into one or both of:
a. an endogenous T cell receptor alpha subunit constant gene (TRAC), and
b. an endogenous T cell receptor beta subunit constant gene (TRBC),
   wherein the at least one heterologous gene comprises at least one of:
a. a variable region of a heterologous human TCR-α chain gene, and
b. a variable region of a heterologous human TCR-β chain gene.

Embodiment 14. The engineered cell of any one of embodiments 9-13, wherein the at least one nucleic acid sequence does not comprise a viral vector.

Embodiment 15. The engineered cell of any one of embodiments 9-14, wherein the T cell, which is optionally an iPSC-derived T cell, or iPSC cell comprises at least one nucleic acid sequence comprising at least one heterologous gene non-virally inserted into one or both of:

a an endogenous T cell receptor alpha subunit constant gene (TRAC), and
b. an endogenous T cell receptor beta subunit constant gene (TRBC),
   wherein the at least one heterologous gene comprises at least one of:
a. a variable region of a heterologous human TCR-α chain gene, and
b. a variable region of a heterologous human TCR-β chain gene.

Embodiment 16. The engineered cell of any one of embodiments 9-15, wherein the nucleic acid comprises a heterologous TCR-alpha subunit chain and a heterologous TCR-beta subunit chain.

Embodiment 17. The engineered cell of any one of embodiments 9-16, wherein the nucleic acid is inserted into the endogenous TRAC and the endogenous TRBC is deleted.

Embodiment 18. The engineered cell of any one of embodiments 9-17, wherein the nucleic acid comprises the variable region and constant region of a heterologous human TCR-β chain gene and the variable region of a heterologous human TCR-α chain gene.

Embodiment 19. The engineered cell of any one of embodiments 9-18, wherein the nucleic acid comprises, from N-terminus to C-terminus:
a. a first self-cleaving peptide sequence;
b. the variable region and constant region of a heterologous human TCR-β chain gene;
c. a second self-cleaving peptide sequence;
d. the variable region of a heterologous human TCR-α chain gene; and
e. a portion of the N-terminus of the endogenous TRAC.

Embodiment 20. The engineered cell of any one of embodiments 9-19, wherein the at least one heterologous gene replaces a placeholder TCR variable region.

Embodiment 21. A pharmaceutical composition comprising the engineered T cells of any one of embodiments 9-20.

Embodiment 22. A method for treating cancer in a subject in need thereof, comprising administering to the subject a therapeutically effective amount of the engineered T cell of any one of embodiments 9-20 or the pharmaceutical composition of embodiment 21.

Embodiment 23. The method of embodiment 22, wherein the subject is HLA-A*11:01 positive.

Embodiment 24. The method of embodiment 22 or 23, wherein the subject received prior therapy for treating the cancer.

Embodiment 25. The method of any one of embodiments 22-24, wherein the cancer is locally advanced, unresectable, metastatic, refractory, or recurrent cancer.

Embodiment 26. The method of any one of embodiments 22-25, wherein engineered T cells are administered at a dose of ≥7.5×10⁸ cells and ≤4.5×10¹⁰ cells.

Embodiment 27. The method of any one of embodiments 22-26, wherein the engineered T cells are administered via intravenous infusion.

Embodiment 28. The method of any one of embodiments 22-27 wherein the cancer is selected from the group consisting of: pancreatic cancer (e.g., pancreatic ductal adenocarcinoma (PDAC)), colorectal cancer (CRC), lung cancer (e.g., non-small cell lung cancer).

Embodiment 29. The method of any one of embodiments 22-28, further comprising administering an anti-PD-L1 antibody.

Embodiment 30. The method of embodiment 29, wherein the anti-PD-L1 antibody is atezolizumab.

Embodiment 31. The method of any one of embodiments 22-30, wherein the method further comprises administering to the subject a lymphodepleting chemotherapy regimen prior to administration of the engineered T cells.

Embodiment 32. The method of embodiment 31, wherein the lymphodepleting chemotherapy regimen comprises fludarabine and cyclophosphamide.

Embodiment 33. A recombinant T cell receptor (TCR) that binds to a KRAS G12D neoantigen, comprising a TCR-alpha chain variable region and a TCR-beta chain variable region, wherein
the TCR-alpha chain variable region comprises:
a. a CDR1 sequence comprising an amino acid sequence set forth in SEQ ID NOs: 19, 29, 39, 49, 59, 69, 79, or 89; and
b. a CDR2 sequence comprising an amino acid sequence set forth in SEQ ID NOs: 20, 30, 40, 50, 60, 70, 80, or 90; and
c. a CDR3 sequence comprising an amino acid sequence set forth in SEQ ID NOs: 21, 31, 41, 51, 61, 71, 81, or 91; and
the TCR-beta chain variable region comprises:
a. a CDR1 sequence comprising an amino acid sequence set forth in SEQ ID NOs: 16, 26, 36, 46, 56, 66, 76, or 86; and
b. a CDR2 sequence comprising an amino acid sequence set forth in SEQ ID NOs: 17, 27, 37, 47, 57, 67, 77, or 87; and
c. a CDR3 sequence comprising an amino acid sequence set forth in SEQ ID NOs: 18, 28, 38, 48, 58, 68, 78, or 88.

Embodiment 34. The recombinant TCR of embodiment 33, wherein the TCR comprises:
a. a TCR-alpha chain variable region comprising a CDR1, a CDR2, and a CDR3 comprising amino acid sequences SEQ ID NOs: 19, 20, and 21, respectively, and a TCR-beta chain variable region comprising a CDR1, a CDR2, and a CDR3 comprising amino acid sequences SEQ ID NOs: 16, 17, and 18, respectively; or
b. a TCR-alpha chain variable region comprising a CDR1, a CDR2, and a CDR3 comprising amino acid sequences SEQ ID NOs: 29, 30, and 31, respectively, and a TCR-beta chain variable region comprising a CDR1, a CDR2, and a CDR3 comprising amino acid sequences SEQ ID NOs: 26, 27, and 28, respectively; or
c. a TCR-alpha chain variable region comprising a CDR1, a CDR2, and a CDR3 comprising amino acid sequences SEQ ID NOs: 39, 40, and 41, respectively, and a TCR-beta chain variable region comprising a CDR1, a CDR2, and a CDR3 comprising amino acid sequences SEQ ID NOs: 36, 37, and 38, respectively; or
d. a TCR-alpha chain variable region comprising a CDR1, a CDR2, and a CDR3 comprising amino acid sequences SEQ ID NOs: 49, 50, and 51, respectively, and a TCR-beta chain variable region comprising a CDR1, a CDR2, and a CDR3 comprising amino acid sequences SEQ ID NOs: 46, 47, and 48, respectively; or
e. a TCR-alpha chain variable region comprising a CDR1, a CDR2, and a CDR3 comprising amino acid sequences SEQ ID NOs: 59, 60, and 61, respectively, and a TCR-beta chain variable region comprising a CDR1, a CDR2, and a CDR3 comprising amino acid sequences SEQ ID NOs: 56, 57, and 58, respectively; or
f. a TCR-alpha chain variable region comprising a CDR1, a CDR2, and a CDR3 comprising amino acid sequences SEQ ID NOs: 69, 70, and 71, respectively, and a TCR-beta chain variable region comprising a CDR1, a CDR2, and a CDR3 comprising amino acid sequences SEQ ID NOs: 66, 67, and 68, respectively; or
g. a TCR-alpha chain variable region comprising a CDR1, a CDR2, and a CDR3 comprising amino acid sequences SEQ ID NOs: 79, 80, and 81, respectively, and a TCR-beta chain variable region comprising a CDR1, a CDR2, and a CDR3 comprising amino acid sequences SEQ ID NOs: 76, 77, and 78, respectively; or
h. a TCR-alpha chain variable region comprising a CDR1, a CDR2, and a CDR3 comprising amino acid sequences SEQ ID NOs: 89, 90, and 91, respectively, and a TCR-beta chain variable region comprising a CDR1, a CDR2, and a CDR3 comprising amino acid sequences SEQ ID NOs: 86, 87, and 88, respectively.

Embodiment 35. The recombinant TCR of embodiment 33 or 34, wherein the TCR-alpha chain variable region comprises at least 95%, at least 96%, at least 97%, at least 98%, at least 99%, or 100% sequence identity to the amino acid sequence set forth in SEQ ID NO: 22, 32, 42, 52, 62, 72, 82, or 92.

Embodiment 36. The recombinant TCR of any one of embodiments 33-35, wherein the TCR-beta chain variable region comprises at least 95%, at least 96%, at least 97%, at least 98%, at least 99%, or 100% sequence identity to the amino acid sequence set forth in SEQ ID NO: 23, 33, 43, 53, 63, 73, 83, or 93.

Embodiment 37. The recombinant TCR of any one of embodiments 33-36, comprising:
a. a TCR-alpha chain variable region and a TCR-beta chain variable region having at least 95%, at least 96%, at least 97%, at least 98%, at least 99%, or 100% sequence identity to SEQ ID NO: 22 and SEQ ID: 23, respectively; or
b. a TCR-alpha chain variable region and a TCR-beta chain variable region having at least 95%, at least 96%, at least 97%, at least 98%, at least 99%, or 100% sequence identity to SEQ ID NO: 32 and SEQ ID: 33, respectively; or
c. a TCR-alpha chain variable region and a TCR-beta chain variable region having at least 95%, at least 96%, at least 97%, at least 98%, at least 99%, or 100% sequence identity to SEQ ID NO: 42 and SEQ ID: 43, respectively; or
d. a TCR-alpha chain variable region and a TCR-beta chain variable region having at least 95%, at least 96%, at least 97%, at least 98%, at least 99%, or 100% sequence identity to SEQ ID NO: 52 and SEQ ID: 53, respectively; or
e. a TCR-alpha chain variable region and a TCR-beta chain variable region having at least 95%, at least 96%, at least 97%, at least 98%, at least 99%, or 100% sequence identity to SEQ ID NO: 62 and SEQ ID: 63, respectively; or
f. a TCR-alpha chain variable region and a TCR-beta chain variable region having at least 95%, at least 96%, at least 97%, at least 98%, at least 99%, or 100% sequence identity to SEQ ID NO: 72 and SEQ ID: 73, respectively; or
g. a TCR-alpha chain variable region and a TCR-beta chain variable region having at least 95%, at least 96%, at least 97%, at least 98%, at least 99%, or 100% sequence identity to SEQ ID NO: 82 and SEQ ID: 83, respectively; or
h. a TCR-alpha chain variable region and a TCR-beta chain variable region having at least 95%, at least 96%, at least 97%, at least 98%, at least 99%, or 100% sequence identity to SEQ ID NO: 92 and SEQ ID: 93, respectively.

Embodiment 38. The recombinant TCR of any one of embodiments 33-37, comprising:
a. a TCR-alpha chain and a TCR-beta chain having at least 95%, at least 96%, at least 97%, at least 98%, at least 99%, or 100% sequence identity to SEQ ID NO: 24 and SEQ ID: 25, respectively; or
b. a TCR-alpha chain and a TCR-beta chain having at least 95%, at least 96%, at least 97%, at least 98%, at least 99%, or 100% sequence identity to SEQ ID NO: 34 and SEQ ID: 35, respectively; or
c. a TCR-alpha chain and a TCR-beta chain having at least 95%, at least 96%, at least 97%, at least 98%, at least 99%, or 100% sequence identity to SEQ ID NO: 44 and SEQ ID: 45, respectively; or
d. a TCR-alpha chain and a TCR-beta chain having at least 95%, at least 96%, at least 97%, at least 98%, at least 99%, or 100% sequence identity to SEQ ID NO: 54 and SEQ ID: 55, respectively; or
e. a TCR-alpha chain and a TCR-beta chain having at least 95%, at least 96%, at least 97%, at least 98%, at least 99%, or 100% sequence identity to SEQ ID NO: 64 and SEQ ID: 65, respectively; or
f. a TCR-alpha chain and a TCR-beta chain having at least 95%, at least 96%, at least 97%, at least 98%, at least 99%, or 100% sequence identity to SEQ ID NO: 74 and SEQ ID: 75, respectively; or
g. a TCR-alpha chain and a TCR-beta chain having at least 95%, at least 96%, at least 97%, at least 98%, at least 99%, or 100% sequence identity to SEQ ID NO: 84 and SEQ ID: 85, respectively; or
h. a TCR-alpha chain and a TCR-beta chain having at least 95%, at least 96%, at least 97%, at least 98%, at least 99%, or 100% sequence identity to SEQ ID NO: 94 and SEQ ID: 95, respectively.

Embodiment 39. The recombinant TCR of any one of embodiments 33-38, wherein the TCR binds the KRAS G12D neoantigen in a subject who is HLA-A*11:01 positive.

Embodiment 40. The recombinant TCR of any one of embodiments 33-39, wherein the TCR is HLA 11:01 restricted.

Embodiment 41. The recombinant TCR of any one of embodiments 33-40,

wherein the TCR activates a T cell upon binding to the KRAS G12D neoantigen.

Embodiment 42. The recombinant TCR of any one of embodiments 33-41, wherein the neoantigen comprises SEQ ID NO: 1 or SEQ ID NO: 2.

Embodiment 43. An engineered cell that expresses the recombinant TCR of any one of embodiments 33-42.

Embodiment 44. The engineered cell of embodiment 43 wherein the cell is an iPSC-derived T cell, a patient-derived autologous T cell, a donor-derived T cell, or an iPSC cell, optionally wherein the patient-derived autologous T cell, donor-derived T cell, or iPSC-derived T cell is a CD8+ T cell.

Embodiment 45. The engineered cell of embodiment 43 or 44 wherein the cell is an iPSC-derived T cell.

Embodiment 46. The engineered cell of embodiment 43 or 44 wherein the cell is a patient-derived autologous T cell.

Embodiment 47. The engineered cell of any one of embodiments 43-46, wherein the T cell, which is optionally an iPSC-derived T cell, or iPSC cell comprises at least one nucleic acid sequence comprising at least one heterologous gene inserted into one or both of:
a. an endogenous T cell receptor alpha subunit constant gene (TRAC), and
b. an endogenous T cell receptor beta subunit constant gene (TRBC),
   wherein the at least one heterologous gene comprises at least one of:
a. a variable region of a heterologous human TCR-α chain gene, and
b. a variable region of a heterologous human TCR-β chain gene.

Embodiment 48. The engineered cell of embodiment 43-47, wherein the at least one nucleic acid sequence does not comprise a viral vector.

Embodiment 49. The engineered cell of embodiment 43-48, wherein the T cell, which is optionally an iPSC-derived T cell, or iPSC cell comprises at least one nucleic acid sequence comprising at least one heterologous gene non-virally inserted into one or both of:
a. an endogenous T cell receptor alpha subunit constant gene (TRAC), and
b. an endogenous T cell receptor beta subunit constant gene (TRBC),
   wherein the at least one heterologous gene comprises at least one of:
a. a variable region of a heterologous human TCR-α chain gene, and
b. a variable region of a heterologous human TCR-β chain gene.

Embodiment 50. The engineered cell of embodiment 43-49, wherein the nucleic acid comprises a heterologous TCR-alpha subunit chain and a heterologous TCR-beta subunit chain.

Embodiment 51. The engineered cell of embodiment 43-50, wherein the nucleic acid is inserted into the endogenous TRAC and the endogenous TRBC is deleted.

Embodiment 52. The engineered cell of embodiment 43-51, wherein the nucleic acid comprises the variable region and constant region of a heterologous human TCR-β chain gene and the variable region of a heterologous human TCR-α chain gene.

Embodiment 53. The engineered cell of embodiment 43-52, wherein the nucleic acid comprises, from N-terminus to C-terminus:
a. a first self-cleaving peptide sequence;
b. the variable region and constant region of a heterologous human TCR-β chain gene;
C. a second self-cleaving peptide sequence;
d. the variable region of a heterologous human TCR-α chain gene; and
e. a portion of the N-terminus of the endogenous TRAC.

Embodiment 54. The engineered cell of embodiment 43-53, wherein the at least one heterologous gene replaces a placeholder TCR variable region.

Embodiment 55. A pharmaceutical composition comprising the engineered T cells of any one of embodiments 43-54.

Embodiment 56. A method for treating cancer in a subject in need thereof, comprising administering to the subject a therapeutically effective amount of the engineered T cell of any one of embodiments 43-54 or the pharmaceutical composition of embodiment 55.

Embodiment 57. The method of embodiment 56, wherein the subject is HLA-A*11:01 positive.

Embodiment 58. The method of embodiment 56 or 57, wherein the subject received prior therapy for treating the cancer.

Embodiment 59. The method of any one of embodiments 56-58, wherein the cancer is locally advanced, unresectable, metastatic, refractory, or recurrent cancer.

Embodiment 60. The method of any one of embodiments 56-59, wherein engineered T cells are administered at a dose of ≥7.5×10⁸ cells and ≤4.5×10¹⁰ cells.

Embodiment 61. The method of any one of embodiments 56-60, wherein the engineered T cells are administered via intravenous infusion.

Embodiment 62. The method of any one of embodiments 56-61 wherein the cancer is selected from the group consisting of: pancreatic cancer (e.g., pancreatic ductal adenocarcinoma (PDAC)), colorectal cancer (CRC), lung cancer (e.g., non-small cell lung cancer).

Embodiment 63. The method of any one of embodiments 56-62, further comprising administering an anti-PD-L1 antibody.

Embodiment 64. The method of embodiment 62, wherein the anti-PD-L1 antibody is atezolizumab.

Embodiment 65. The method of any one of embodiments 56-64, wherein the method further comprises administering to the subject a lymphodepleting chemotherapy regimen prior to administration of the engineered T cells.

Embodiment 66. The method of embodiment 65, wherein the lymphodepleting chemotherapy regimen comprises fludarabine and cyclophosphamide.

Embodiment 67. An engineered cell that expresses a recombinant TCR comprising:
a TCR-alpha chain comprising a TCR-alpha variable region; and
a TCR-beta chain comprising a TCR-beta variable region;
wherein the TCR-alpha variable region comprises the following:
   a CDR1 comprising the amino acid sequence SEQ ID NO: 7,
   a CDR2 comprising the amino acid sequence SEQ ID NO: 8, and
   a CDR3 comprising amino the acid sequence SEQ ID NO: 9; and
wherein the TCR-beta variable region comprises the following:
   a CDR1 comprising the amino acid sequence SEQ ID NO: 3,
   a CDR2 comprising the amino acid sequence SEQ ID NO: 4, and
   a CDR3 comprising the amino acid sequence SEQ ID NO: 6; and
wherein the T cell, which is optionally an iPSC-derived T cell, or iPSC cell comprises at least one nucleic acid sequence comprising at least one heterologous gene inserted into one or both of:
   a. an endogenous T cell receptor alpha subunit constant gene (TRAC), and
   b. an endogenous T cell receptor beta subunit constant gene (TRBC),
wherein the at least one heterologous gene comprises at least one of:
   a. a variable region of a heterologous human TCR-α chain gene, and
   b. a variable region of a heterologous human TCR-β chain gene; and
wherein intracellular delivery of the at least one heterologous gene comprises introducing the at least one heterologous gene using a viral vector.

Embodiment 68. An engineered cell that expresses a recombinant TCR comprising:
a TCR-alpha chain comprising a TCR-alpha variable region; and
a TCR-beta chain comprising a TCR-beta variable region;
wherein the TCR-alpha chain variable region comprises at least 95%, at least 96%, at least 97%, at least 98%, at least 99%, or 100% sequence identity to the amino acid sequence set forth in SEQ ID NO: 10 and the TCR-beta chain variable region comprises at least 95%, 96%, 97%, 98%, 99%, or 100% sequence identity to the amino acid sequence set forth in SEQ ID NO: 11; and
wherein the T cell, which is optionally an iPSC-derived T cell, or iPSC cell comprises at least one nucleic acid sequence comprising at least one heterologous gene inserted into one or both of:
   a. an endogenous T cell receptor alpha subunit constant gene (TRAC), and
   b. an endogenous T cell receptor beta subunit constant gene (TRBC),
wherein the at least one heterologous gene comprises at least one of:
   a. a variable region of a heterologous human TCR-α chain gene, and
   b. a variable region of a heterologous human TCR-β chain gene; and
wherein intracellular delivery of the at least one heterologous gene comprises introducing the at least one heterologous gene using a viral vector.

Embodiment 69. An engineered cell that expresses a recombinant TCR comprising:
a TCR-alpha chain comprising a TCR-alpha variable region; and
a TCR-beta chain comprising a TCR-beta variable region;
wherein the TCR-alpha chain comprises at least 95%, at least 96%, at least 97%, at least 98%, at least 99%, or 100% sequence identity to the amino acid sequence set forth in SEQ ID NO: 12; and
wherein the TCR-beta chain comprises at least 95%, at least 96%, at least 97%, at least 98%, at least 99%, or 100% sequence identity to the amino acid sequence set forth in SEQ ID NO: 13, SEQ ID NO: 14, or 15; and
wherein the T cell, which is optionally an iPSC-derived T cell, or iPSC cell comprises at least one nucleic acid sequence comprising at least one heterologous gene inserted into one or both of:
   a. an endogenous T cell receptor alpha subunit constant gene (TRAC), and
   b. an endogenous T cell receptor beta subunit constant gene (TRBC),
wherein the at least one heterologous gene comprises at least one of:
   a. a variable region of a heterologous human TCR-α chain gene, and
   b. a variable region of a heterologous human TCR-β chain gene; and
wherein intracellular delivery of the at least one heterologous gene comprises introducing the at least one heterologous gene using a viral vector.

Embodiment 70. A method of preparing the engineered cell of any one of embodiments 67-69 comprising
a. providing a cell, and
b. introducing at least one heterologous gene in a viral vector into the cell,
wherein the viral vector comprises a nucleic acid sequence encoding at least the TCR alpha subunit and/or the TCR beta subunit,
wherein optionally the TCR alpha and/or TCR beta subunits are inserted into the host cell at the TCR alpha locus and/or TCR beta locus, respectively, and/or at a locus outside of the endogenous TCR alpha locus and/or TCR beta locus.

Additional objects and advantages will be set forth in part in the description which follows, and in part will be understood from the description, or may be learned by practice.

The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

The novel features of the invention are set forth with particularity in the appended claims. A better understanding of the features and advantages of the present invention is obtained by reference to the following detailed description that sets forth illustrative embodiments, in which the principles of the invention are utilized, and the accompanying drawings of which:

FIG. 1 shows T cell activation, specifically the percent of T cells expressing CD137 as a marker of T cell activation after incubation with T2/A11 cells that have been pulsed with the KRAS 10-mer mutant peptide (VVVGADGVGK) (SEQ ID NO: 1).

FIG. 2 shows killing of peptide cytolytic activity (as percent lysis), specifically the percent T2/A11 cells lysed by T cells activated after exposure to the KRAS 10-mer mutant peptide (VVVGADGVGK) (SEQ ID NO: 1).

FIG. 3 shows T cell activation measured by percent expressing CD137 (a measure of T cell activation), specifically the percent of T cells expressing CD137 as a marker of T cell activation after incubation with T2/A11 cells that have been pulsed with the KRAS 9-mer mutant peptide (VVGADGVGK) (SEQ ID NO: 2).

FIG. 4 shows killing of peptide loaded targets measured by cytolytic activity (as percent lysis), specifically the percent T2/A11 cells lysed by T cells activated after exposure to the KRAS 9-mer mutant peptide (VVGADGVGK) (SEQ ID NO: 2).

FIG. 5 shows IFN gamma cytokine secretion (another marker of T cell activation) by T cells expressing KRAS-specific TCRs after incubation with T2/A11 cells that have been pulsed with the KRAS 10-mer mutant peptide (VVVGADGVGK) (SEQ ID NO: 1).

FIG. 6 shows IFN gamma secretion by T cells expressing KRAS-specific TCRs after incubation with T2/A11 cells that have been pulsed with the KRAS 9-mer mutant peptide (VVGADGVGK) (SEQ ID NO: 2).

FIG. 7 shows the percent of T cells expressing CD137 as a marker of T cell activation after exposure to SU8686 pancreatic carcinoma cells expressing 10 mer mutant peptide (VVVGADGVGK) (SEQ ID NO: 1). SU8686 cells do not naturally express HLA A*11:01 and FIG. 7 shows cells without added HLA A*11:01 and cells transduced with a viral vector carrying A*11:01.

FIG. 8 shows IFN gamma secretion by T cells expressing KRAS-specific TCRs after incubation with tumor cell lines that were positive for the KRAS G12D mutation and either did or did not express HLA A*11:01. SNU-1 and SU8686 cells express the KRAS G12D mutation.

FIG. 9 shows IFN gamma secretion by T cells expressing KRAS-specific TCRs after incubation with tumor cell lines that either were or were not positive for the KRAS G12D mutation and either did not express HLA A*11:01. FIG. 8 shows cells without added HLA A*11:01 and cells transduced with a viral vector carrying A*11:01.SNU1 expresses the KRAS G12D mutation, while the following tumor cell lines do not: NCIH1373, RKN, CFPAC1, NCIH727, and SW527.

FIG. 10 shows T cell activation in response to gastric carcinoma cells harboring the KRAS-p.G12D mutation (+/−HLA A*11:01) that were not pulsed with peptide, were pulsed with a 10-mer wild-type KRAS peptide, or were pulsed with the 10-mer mutant KRAS G12D peptide (VVVGADGVGK) (SEQ ID NO: 1).

FIG. 11 shows T cell activation in response to gastric carcinoma cells harboring the KRAS-p.G12D mutation (+/−HLA A*11:01) that were not pulsed with peptide, were pulsed with a 9-mer wild-type KRAS peptide, or were pulsed with the 9-mer mutant KRAS G12D peptide (VVGADGVGK) (SEQ ID NO: 2).

FIG. 12A-B show the percentage of CD137 upregulation by CD8+ T cells expressing the KRAS G12D-specific EE209_2 TCR following stimulation with increasing concentrations of the FIG. 12A 10-mer or FIG. 12B 9-mer KRAS G12D peptide. The frequency of CD137+ cells was normalized to the frequency of cells expressing the EE209_2 TCR. Circles with different grayscale represent individual donors.

FIG. 13A-B show the percent specific lysis of HLA-A*11:01+target B cells by CD8+ T cells expressing the KRAS G12D-specific EE209_2 TCR in the presence of increasing concentrations of the FIG. 13A 10-mer or FIG. 13B 9-mer KRAS G12D peptide. Circles with different grayscale represent individual donors.

FIG. 14A-D shows the production of FIG. 14A-B Granzyme B and FIG. 14C-FIG. 14D IFNγ by CD8+ T cells expressing the KRAS G12D-specific EE209_2 TCR following stimulation with increasing concentrations of the FIG. 14A, FIG. 14C 10-mer or FIG. 14B, FIG. 14D 9-mer KRAS G12D peptides. *=data points outside of the linear range of the standard curves. Circles with different grayscale represent individual donors.

FIG. 15A-D shows FIG. 15A target cell lysis, FIG. 15B upregulation of CD137, normalized to TCR knock-in efficiency, FIG. 15C Granzyme B production, and FIG. 15D IFNγ production by CD8+ T cells expressing the KRAS G12D-specific EE209_2 TCR following stimulation with increasing concentrations of the 10-mer wildtype KRAS peptide. Circles with different grayscale represent individual donors.

FIG. 16A-D shows FIG. 16A target cell lysis, FIG. 16B upregulation of CD137, normalized to TCR knock-in efficiency, FIG. 16C granzyme B production, and FIG. 16D IFNγ production by CD8+ T cells expressing the KRAS G12D-specific EE209_2 TCR following stimulation with increasing concentrations of the 9-mer wildtype KRAS peptide. Circles with different grayscale represent individual donors.

FIG. 17A-B show killing of HLA-A*11:01+KRAS G12D-mutant tumor cell lines by KRAS G12D TCR KI T cells. FIG. 17A showing percentage killing at varying E:T ratios. The number of effector cells were not adjusted for TCR KI. Negative values represent increased growth of tumor cells in coculture conditions relative to tumor-only control wells. FIG. 17B shows percentage killing of HLA-A*11:01+KRAS G12D-mutant tumor cell lines pulsed with 10 μM 10-mer KRAS G12D peptide at E:T ratio=9:1.

FIG. 18A-C shows T cell activation and cytokine secretion in the presence of HLA-A*11:01+KRAS G12D-mutant tumor cell lines with FIG. 18A showing the percentage of CD137 upregulation by KRAS G12D TCR KI T cells (values are normalized to the percentage of TCR KI cells), FIG. 18B showing granzyme B levels in culture supernatants, and FIG. 18C showing IFNγ levels detected in culture supernatants.

FIG. 19A-C shows T cell activation and cytokine secretion in the presence of HLA-A*11:01+KRAS G12D-mutant tumor cell lines with FIG. 19A showing the percentage of CD137 upregulation by KRAS G12D TCR KI T cells (values are normalized to the percentage of TCR KI cells), FIG. 19B showing granzyme B levels in culture supernatants, and (C) IFNγ levels detected in culture supernatants.

FIG. 20 shows the study schema for Phase Ia (dose escalation and expansion as a monotherapy) and Phase Ib (safety run-in and expansion in combination with atezolizumab) of a Phase I clinical trial.

FIG. 21A-B shows a T cell engineering process with FIG. 21A showing knock-in of the Neo TCR (KRAS G12D-specific TCR) into an endogenous TCR-locus locus, and FIG. 21B showing knock-out of an endogenous TCR-beta locus.

### DETAILED DESCRIPTION

The practice of the present invention may employ, unless otherwise indicated, conventional techniques and descriptions of molecular biology, bioinformatics, cell biology, and biochemistry, which are within the skill of the art. Such conventional techniques include, but are not limited to, sampling and analysis of blood cells, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals. All references, articles, publications, patents, patent publications, and patent applications cited herein are incorporated by reference in their entireties for all purposes as though fully set forth herein.

The present invention includes, in some embodiments, a recombinant T cell receptor (TCR) that binds to a Kirsten rat sarcoma viral oncogene homolog (KRAS) G12D neoantigen comprising: a TCR-alpha chain comprising a TCR-alpha variable region; and a TCR-beta chain comprising a TCR-beta variable region; wherein the TCR-alpha variable region comprises the following: a CDR1 comprising the amino acid sequence SEQ ID NO: 7, a CDR2 comprising the amino acid sequence SEQ ID NO: 8, and a CDR3 comprising amino the acid sequence SEQ ID NO: 9; and wherein the TCR-beta variable region comprises the following: a CDR1 comprising the amino acid sequence SEQ ID NO: 3, a CDR2 comprising the amino acid sequence SEQ ID NO: 4, and a CDR3 comprising the amino acid sequence SEQ ID NO: 6. In some embodiments, the TCR-alpha chain variable region comprises at least 95%, at least 96%, at least 97%, at least 98%, at least 99%, or 100% sequence identity to the amino acid sequence set forth in SEQ ID NO: 10 and the TCR-beta chain variable region comprises at least 95%, 96%, 97%, 98%, 99%, or 100% sequence identity to the amino acid sequence set forth in SEQ ID NO: 11. In some embodiments, the TCR-alpha chain comprises at least 95%, at least 96%, at least 97%, at least 98%, at least 99%, or 100% sequence identity to the amino acid sequence set forth in SEQ ID NO: 12. In some embodiments, the TCR-beta chain comprises at least 95%, at least 96%, at least 97%, at least 98%, at least 99%, or 100% sequence identity to the amino acid sequence set forth in SEQ ID NO: 13, SEQ ID NO: 14, or 15.

The present invention includes, in some embodiments, a recombinant T cell receptor (TCR) that binds to a KRAS G12D neoantigen, comprising a TCR-alpha chain variable region and a TCR-beta chain variable region, wherein the TCR-alpha chain variable region comprises: a CDR1 sequence comprising an amino acid sequence set forth in SEQ ID NOs: 19, 29, 39, 49, 59, 69, 79, or 89; and a CDR2 sequence comprising an amino acid sequence set forth in SEQ ID NOs: 20, 30, 40, 50, 60, 70, 80, or 90; and a CDR3 sequence comprising an amino acid sequence set forth in SEQ ID NOs: 21, 31, 41, 51, 61, 71, 81, or 91; and the beta chain variable region comprises: a CDR1 sequence comprising an amino acid sequence set forth in SEQ ID NOs: 16, 26, 36, 46, 56, 66, 76, or 86; and a CDR2 sequence comprising an amino acid sequence set forth in SEQ ID NOs: 17, 27, 37, 47, 57, 67, 77, or 87; and a CDR3 sequence comprising an amino acid sequence set forth in SEQ ID NOs: 18, 28, 38, 48, 58, 68, 78, or 88. In some embodiments, the TCR comprises: a TCR-alpha chain variable region comprising a CDR1, a CDR2, and a CDR3 comprising amino acid sequences SEQ ID NOs: 19, 20, and 21, respectively, and a TCR-beta chain variable region comprising a CDR1, a CDR2, and a CDR3 comprising amino acid sequences SEQ ID NOs: 16, 17, and 18, respectively; or a TCR-alpha chain variable region comprising a CDR1, a CDR2, and a CDR3 comprising amino acid sequences SEQ ID NOs: 29, 30, and 31, respectively, and a TCR-beta chain variable region comprising a CDR1, a CDR2, and a CDR3 comprising amino acid sequences SEQ ID NOs: 26, 27, and 28, respectively; or a TCR-alpha chain variable region comprising a CDR1, a CDR2, and a CDR3 comprising amino acid sequences SEQ ID NOs: 39, 40, and 41, respectively, and a TCR-beta chain variable region comprising a CDR1, a CDR2, and a CDR3 comprising amino acid sequences SEQ ID NOs: 36, 37, and 38, respectively; or a TCR-alpha chain variable region comprising a CDR1, a CDR2, and a CDR3 comprising amino acid sequences SEQ ID NOs: 49, 50, and 51, respectively, and a TCR-beta chain variable region comprising a CDR1, a CDR2, and a CDR3 comprising amino acid sequences SEQ ID NOs: 46, 47, and 48, respectively; or a TCR-alpha chain variable region comprising a CDR1, a CDR2, and a CDR3 comprising amino acid sequences SEQ ID NOs: 59, 60, and 61, respectively, and a TCR-beta chain variable region comprising a CDR1, a CDR2, and a CDR3 comprising amino acid sequences SEQ ID NOs: 56, 57, and 58, respectively; or a TCR-alpha chain variable region comprising a CDR1, a CDR2, and a CDR3 comprising amino acid sequences SEQ ID NOs: 69, 70, and 71, respectively, and a TCR-beta chain variable region comprising a CDR1, a CDR2, and a CDR3 comprising amino acid sequences SEQ ID NOs: 66, 67, and 68, respectively; or a TCR-alpha chain variable region comprising a CDR1, a CDR2, and a CDR3 comprising amino acid sequences SEQ ID NOs: 79, 80, and 81, respectively, and a TCR-beta chain variable region comprising a CDR1, a CDR2, and a CDR3 comprising amino acid sequences SEQ ID NOs: 76, 77, and 78, respectively; or a TCR-alpha chain variable region comprising a CDR1, a CDR2, and a CDR3 comprising amino acid sequences SEQ ID NOs: 89, 90, and 91, respectively, and a TCR-beta chain variable region comprising a CDR1, a CDR2, and a CDR3 comprising amino acid sequences SEQ ID NOs: 86, 87, and 88, respectively. In some embodiments, the TCR-alpha chain variable region comprises at least 95%, at least 96%, at least 97%, at least 98%, at least 99%, or 100% sequence identity to the amino acid sequence set forth in SEQ ID NO: 22, 32, 42, 52, 62, 72, 82, or 92. In some embodiments, the TCR-beta chain variable region comprises at least 95%, at least 96%, at least 97%, at least 98%, at least 99%, or 100% sequence identity to the amino acid sequence set forth in SEQ ID NO: 23, 33, 43, 53, 63, 73, 83, or 93.

In some embodiments, the recombinant TCR comprises a TCR-alpha chain variable region and a TCR-beta chain variable region having at least 95%, at least 96%, at least 97%, at least 98%, at least 99%, or 100% sequence identity to SEQ ID NO: 22 and SEQ ID: 23, respectively; or a TCR-alpha chain variable region and a TCR-beta chain variable region having at least 95%, at least 96%, at least 97%, at least 98%, at least 99%, or 100% sequence identity to SEQ ID NO: 32 and SEQ ID: 33, respectively; or a TCR-alpha chain variable region and a TCR-beta chain variable region having at least 95%, at least 96%, at least 97%, at least 98%, at least 99%, or 100% sequence identity to SEQ ID NO: 42 and SEQ ID: 43, respectively; or a TCR-alpha chain variable region and a TCR-beta chain variable region having at least 95%, at least 96%, at least 97%, at least 98%, at least 99%, or 100% sequence identity to SEQ ID NO: 52 and SEQ ID: 53, respectively; or a TCR-alpha chain variable region and a TCR-beta chain variable region having at least 95%, at least 96%, at least 97%, at least 98%, at least 99%, or 100% sequence identity to SEQ ID NO: 62 and SEQ ID: 63, respectively; or a TCR-alpha chain variable region and a TCR-beta chain variable region having at least 95%, at least 96%, at least 97%, at least 98%, at least 99%, or 100% sequence identity to SEQ ID NO: 72 and SEQ ID: 73, respectively; or a TCR-alpha chain variable region and a TCR-beta chain variable region having at least 95%, at least 96%, at least 97%, at least 98%, at least 99%, or 100% sequence identity to SEQ ID NO: 82 and SEQ ID: 83, respectively; or a TCR-alpha chain variable region and a TCR-beta chain variable region having at least 95%, at least 96%, at least 97%, at least 98%, at least 99%, or 100% sequence identity to SEQ ID NO: 92 and SEQ ID: 93, respectively. The recombinant TCR of some embodiments comprises: a TCR-alpha chain and a TCR-beta chain having at least 95%, at least 96%, at least 97%, at least 98%, at least 99%, or 100% sequence identity to SEQ ID NO: 24 and SEQ ID: 25, respectively; or a TCR-alpha chain and a TCR-beta chain having at least 95%, at least 96%, at least 97%, at least 98%, at least 99%, or 100% sequence identity to SEQ ID NO: 34 and SEQ ID: 35, respectively; or a TCR-alpha chain and a TCR-beta chain having at least 95%, at least 96%, at least 97%, at least 98%, at least 99%, or 100% sequence identity to SEQ ID NO: 44 and SEQ ID: 45, respectively; or a TCR-alpha chain and a TCR-beta chain having at least 95%, at least 96%, at least 97%, at least 98%, at least 99%, or 100% sequence identity to SEQ ID NO: 54 and SEQ ID: 55, respectively; or a TCR-alpha chain and a TCR-beta chain having at least 95%, at least 96%, at least 97%, at least 98%, at least 99%, or 100% sequence identity to SEQ ID NO: 64 and SEQ ID: 65, respectively; or a TCR-alpha chain and a TCR-beta chain having at least 95%, at least 96%, at least 97%, at least 98%, at least 99%, or 100% sequence identity to SEQ ID NO: 74 and SEQ ID: 75, respectively; or a TCR-alpha chain and a TCR-beta chain having at least 95%, at least 96%, at least 97%, at least 98%, at least 99%, or 100% sequence identity to SEQ ID NO: 84 and SEQ ID: 85, respectively; or a TCR-alpha chain and a TCR-beta chain having at least 95%, at least 96%, at least 97%, at least 98%, at least 99%, or 100% sequence identity to SEQ ID NO: 94 and SEQ ID: 95, respectively.

In some embodiments, the recombinant TCR binds the KRAS G12D neoantigen in a subject who is HLA-A*11:01 positive. In some embodiments, the TCR is HLA 11:01 restricted. In some embodiments, the TCR activates a T cell upon binding to (i.e., recognition of) the KRAS G12D neoantigen. In some embodiments of the invention, the neoantigen to which the recombinant TCR binds comprises the amino acid sequence SEQ ID NO: 1 or SEQ ID NO: 2.

Some embodiments of the present include an engineered cell that expresses any of the recombinant TCRs of the invention. In some embodiments, the cell is an iPSC-derived T cell, a patient-derived autologous T cell, a donor-derived T cell, or an iPSC cell, optionally wherein the patient-derived autologous T cell, donor-derived T cell, or iPSC-derived T cell is a CD8+ T cell. In some embodiments, the cell is an iPSC-derived T cell. In some embodiments, the cell is a patient-derived autologous T cell. In some embodiments, the T cell, which is optionally an iPSC-derived T cell, or iPSC cell comprises at least one nucleic acid sequence comprising at least one heterologous gene inserted into one or both of: an endogenous T cell receptor alpha subunit constant gene (TRAC), and an endogenous T cell receptor beta subunit constant gene (TRBC), wherein the at least one heterologous gene comprises at least one of: a variable region of a heterologous human TCR-α chain gene, and a variable region of a heterologous human TCR-β chain gene. In some embodiments, the at least one nucleic acid sequence does not comprise a viral vector. In some embodiments, the T cell, which is optionally an iPSC-derived T cell, or iPSC cell comprises at least one nucleic acid sequence comprising at least one heterologous gene non-virally inserted into one or both of: an endogenous T cell receptor alpha subunit constant gene (TRAC), and an endogenous T cell receptor beta subunit constant gene (TRBC), wherein the at least one heterologous gene comprises at least one of: a variable region of a heterologous human TCR-α chain gene, and a variable region of a heterologous human TCR-β chain gene. In some embodiments, the nucleic acid comprises a heterologous TCR-alpha subunit chain and a heterologous TCR-beta subunit chain. In some embodiments, the nucleic acid is inserted into the endogenous TRAC and the endogenous TRBC is deleted. In some embodiments, the nucleic acid is inserted into an endogenous TRBC gene and the endogenous TRAC is deleted. In some embodiments, the nucleic acid comprises the variable region and constant region of a heterologous human TCR-β chain gene and the variable region of a heterologous human TCR-α chain gene. In some embodiments, the nucleic acid comprises, from N-terminus to C-terminus: a first self-cleaving peptide sequence; the variable region and constant region of a heterologous human TCR-β chain gene; a second self-cleaving peptide sequence; the variable region of a heterologous human TCR-α chain gene; and a portion of the N-terminus of the endogenous TRAC. In some embodiments, the at least one heterologous gene replaces a placeholder TCR variable region. In some embodiments, the nucleic acid comprises any DNA sequence that encodes the recited amino acid sequences.

In some embodiments, the recombinant TCR is inserted into a T cell through use of a viral vector encoding the recombinant TCR. In some embodiments, the engineered cell that expresses the recombinant TCR comprises a TCR-alpha chain comprising a TCR-alpha variable region; and a TCR-beta chain comprising a TCR-beta variable region; wherein the TCR-alpha variable region comprises the following: a CDR1 comprising the amino acid sequence SEQ ID NO: 7, a CDR2 comprising the amino acid sequence SEQ ID NO: 8, and a CDR3 comprising amino the acid sequence SEQ ID NO: 9; and wherein the TCR-beta variable region comprises the following: a CDR1 comprising the amino acid sequence SEQ ID NO: 3, a CDR2 comprising the amino acid sequence SEQ ID NO: 4, and a CDR3 comprising the amino acid sequence SEQ ID NO: 6; and wherein the T cell, which is optionally an iPSC-derived T cell, or iPSC cell comprises at least one nucleic acid sequence comprising at least one heterologous gene inserted into one or both of: an endogenous T cell receptor alpha subunit constant gene (TRAC), and an endogenous T cell receptor beta subunit constant gene (TRBC), wherein the at least one heterologous gene comprises at least one of: a variable region of a heterologous human TCR-α chain gene, and a variable region of a heterologous human TCR-chain gene; and wherein intracellular delivery of the at least one heterologous gene comprises introducing the at least one heterologous gene using a viral vector. In some embodiments, the engineered cell that expresses the recombinant TCR comprises a TCR-alpha chain comprising a TCR-alpha variable region; and a TCR-beta chain comprising a TCR-beta variable region; wherein the TCR-alpha chain variable region comprises at least 95%, at least 96%, at least 97%, at least 98%, at least 99%, or 100% sequence identity to the amino acid sequence set forth in SEQ ID NO: 10 and the TCR-beta chain variable region comprises at least 95%, 96%, 97%, 98%, 99%, or 100% sequence identity to the amino acid sequence set forth in SEQ ID NO: 11; and wherein the T cell, which is optionally an iPSC-derived T cell, or iPSC cell comprises at least one nucleic acid sequence comprising at least one heterologous gene inserted into one or both of: an endogenous T cell receptor alpha subunit constant gene (TRAC), and an endogenous T cell receptor beta subunit constant gene (TRBC), wherein the at least one heterologous gene comprises at least one of: a variable region of a heterologous human TCR-α chain gene, and a variable region of a heterologous human TCR-β chain gene; and wherein intracellular delivery of the at least one heterologous gene comprises introducing the at least one heterologous gene using a viral vector. In some embodiments, the engineered cell that expresses the recombinant TCR comprises a TCR-alpha chain comprising a TCR-alpha variable region; and a TCR-beta chain comprising a TCR-beta variable region; wherein the TCR-alpha chain comprises at least 95%, at least 96%, at least 97%, at least 98%, at least 99%, or 100% sequence identity to the amino acid sequence set forth in SEQ ID NO: 12; and wherein the TCR-beta chain comprises at least 95%, at least 96%, at least 97%, at least 98%, at least 99%, or 100% sequence identity to the amino acid sequence set forth in SEQ ID NO: 13, SEQ ID NO: 14, or 15; and wherein the T cell, which is optionally an iPSC-derived T cell, or iPSC cell comprises at least one nucleic acid sequence comprising at least one heterologous gene inserted into one or both of: an endogenous T cell receptor alpha subunit constant gene (TRAC), and an endogenous T cell receptor beta subunit constant gene (TRBC), wherein the at least one heterologous gene comprises at least one of: a variable region of a heterologous human TCR-α chain gene, and a variable region of a heterologous human TCR-β chain gene; and wherein intracellular delivery of the at least one heterologous gene comprises introducing the at least one heterologous gene using a viral vector. In some embodiments, the engineered cell is prepared using a method comprising (a) providing a cell, and (b) introducing at least one heterologous gene in a viral vector into the cell, wherein the viral vector comprises a nucleic acid sequence encoding at least the TCR alpha subunit and/or the TCR beta subunit, wherein optionally the TCR alpha and/or TCR beta subunits are inserted into the host cell at the TCR alpha locus and/or TCR beta locus, respectively, and/or at a locus outside of the endogenous TCR alpha locus and/or TCR beta locus. In some embodiments, the viral vector comprising the nucleic acid encoding at least the TCR alpha subunit and/or the TCR beta subunit is introduced to the cell through adenoviral, retroviral, or lentiviral transduction. In some embodiments, the engineered cell is prepared using a method comprising (1) providing a cell, and (b) introducing the at least one heterologous gene to the cell using transposase-based engineering, wherein the heterologous gene is inserted into a transposon vector. In some embodiments, the nucleic acid comprises any DNA sequence that encodes the recited amino acid sequences.

Some embodiments of the present invention include a pharmaceutical composition comprising the engineered T cells of any one of embodiments of the present invention.

Some embodiments of the present invention include a method for treating cancer in a subject in need thereof, comprising administering to the subject a therapeutically effective amount of an engineered T cell or the pharmaceutical composition of any embodiment of the invention. In some embodiments, the subject is HLA-A*11:01 positive. In some embodiment, the subject received prior therapy for treating the cancer. In some embodiments, the cancer is locally advanced, unresectable, metastatic, refractory, or recurrent cancer. In some embodiments, engineered T cells are administered at a dose of ≥7.5×10⁸ cells and ≤4.5×10¹⁰ cells. In some embodiments, the engineered T cells are administered via intravenous infusion. In some embodiments, the cancer is selected from the group consisting of: pancreatic cancer (e.g., pancreatic ductal adenocarcinoma (PDAC)), colorectal cancer (CRC), lung cancer (e.g., non-small cell lung cancer). In some embodiments, the method of treating cancer further comprises administering an anti-PD-L1 antibody. In some embodiments, the anti-PD-L1 antibody is atezolizumab. In some embodiments, the method further comprises administering to the subject a lymphodepleting chemotherapy regimen prior to administration of the engineered T cells. In some embodiments, the lymphodepleting chemotherapy regimen comprises fludarabine and cyclophosphamide.

### I. Definitions

“Activation” or “immune activation” or “activated”, especially in reference to T cells, means a phase of an adaptive immune response that follows the antigen recognition phase (during which antigen-specific receptors expressed on the surface of lymphocytes bind to specific antigens or neoantigens) and is characterized by proliferation of lymphocytes and their differentiation into effector cells, e.g. Abbas et al, Cellular and Molecular Immunology, Fourth Edition, (W. B. Saunders Company, 2000). Activation of T cells may be associated with secretion of certain cytokines that are detectable using conventional assays, such as an ELISPOT assay, and may be associated with the expression of characteristic cell surface markers, such as CD25, CD134, CD69, CD137, CD154, or the like, e.g. Gratama et al, Cytometry A, 73A: 971-974 (2008).

“Amplicon” means the product of a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. Preferably, amplicons are formed by the amplification of a single starting sequence. Amplicons may be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26:2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Autologous cell” means a cell that originates from the same subject or patient as the one to which is ultimately transferred in the context of T cell therapy.

“Allogeneic cell” means a cell that originates from a different subject or patient as the one to which it is ultimately transferred in the context of T cell therapy.

The terms “bind,” “binds,” or “binding,” in the context of TCR binding to a neoantigen presented on MCH class I, means “recognizes” and is synonymous with “recognizes.”

“Clonotype” means a recombined nucleotide sequence of a lymphocyte which encodes an immune receptor or a portion thereof. More particularly, clonotype means a recombined nucleotide sequence of a T cell which encodes a T cell receptor (TCR) or a portion thereof. In various embodiments, clonotypes may encode all or a portion of a VDJ rearrangement of TCR beta, a DJ rearrangement of TCR beta, a VJ rearrangement of TCR alpha, a VJ rearrangement of TCR gamma, a VDJ rearrangement of TCR delta, a VD rearrangement of TCR delta, a Kde-V rearrangement, or the like. In one aspect, clonotypes have sequences that are sufficiently long to represent or reflect the diversity of the immune molecules that they are derived from; consequently, clonotypes may vary widely in length. In some embodiments, clonotypes have lengths in the range of from 25 to 400 nucleotides; in other embodiments, clonotypes have lengths in the range of from 25 to 200 nucleotides.

“Clonotype profile” means a listing of distinct clonotypes and their relative abundances that are derived from a population of lymphocytes. Typically, the population of lymphocytes are obtained from a tissue sample. The term “clonotype profile” is related to, but more general than, the immunology concept of immune “repertoire” as described in references, such as the following: Arstila et al, Science, 286:958-961 (1999); Yassai et al, Immunogenetics, 61:493-502 (2009); Kedzierska et al, Mol. Immunol., 45 (3): 607-618 (2008); and the like. The term “clonotype profile” includes a wide variety of lists and abundances of rearranged immune receptor-encoding nucleic acids, which may be derived from selected subsets of lymphocytes (e.g. tissue-infiltrating lymphocytes, immunophenotypic subsets, or the like), or which may encode portions of immune receptors that have reduced diversity as compared to full immune receptors. In some embodiments, clonotype profiles may comprise at least 103 distinct clonotypes; in other embodiments, clonotype profiles may comprise at least 104 distinct clonotypes; in other embodiments, clonotype profiles may comprise at least 105 distinct clonotypes; in other embodiments, clonotype profiles may comprise at least 106 distinct clonotypes. In such embodiments, such clonotype profiles may further comprise abundances or relative frequencies of each of the distinct clonotypes.

In one aspect, a clonotype profile is a set of distinct recombined nucleotide sequences (with their abundances) that encode T cell receptors (TCRs), or fragments thereof, respectively, in a population of lymphocytes of an individual, wherein the nucleotide sequences of the set have a one-to-one correspondence with distinct lymphocytes or their clonal subpopulations for substantially all of the lymphocytes of the population. In one aspect, nucleic acid segments defining clonotypes are selected so that their diversity (i.e. the number of distinct nucleic acid sequences in the set) is large enough so that substantially every T cell or clone thereof in an individual carries a unique nucleic acid sequence of such repertoire. That is, preferably each different clone of a sample has different clonotype. In other aspects of the invention, the population of lymphocytes corresponding to a repertoire may be circulating T cells, or may be subpopulations of either of the foregoing populations, including but not limited to, CD4+ T cells, or CD8+ T cells, or other subpopulations defined by cell surface markers, or the like. Such subpopulations may be acquired by taking samples from particular tissues, e.g. bone marrow, or lymph nodes, or the like, or by sorting or enriching cells from a sample (such as peripheral blood) based on one or more cell surface markers, size, morphology, or the like. In still other aspects, the population of lymphocytes corresponding to a repertoire may be derived from disease tissues, such as a tumor tissue, an infected tissue, or the like. In one embodiment, a clonotype profile comprising human TCR beta chains or fragments thereof comprises a number of distinct nucleotide sequences in the range of from 0.1×10⁶ to 1.8×10⁶, or in the range of from 0.5×10⁶ to 1.5×10⁶, or in the range of from 0.8×10⁶ to 1.2×10⁶.

In a particular embodiment, a clonotype profile of the invention comprises a set of nucleotide sequences that encodes substantially all segments of the V (D) J region of a TCR beta chain. In another embodiment, a clonotype profile of the invention comprises a set of nucleotide sequences having lengths in the range of from 25-200 nucleotides and including segments of the V, D, and J regions of a TCR beta chain. In another embodiment, a clonotype profile of the invention comprises a number of distinct nucleotide sequences that is substantially equivalent to the number of lymphocytes expressing a distinct TCR beta chain. In still another embodiment, “substantially equivalent” means that with ninety-nine percent probability a clonotype profile will include a nucleotide sequence encoding a TCR beta chain or portion thereof carried or expressed by every lymphocyte of a population of an individual at a frequency of 0.001 percent or greater. In still another embodiment, “substantially equivalent” means that with ninety-nine percent probability a repertoire of nucleotide sequences will include a nucleotide sequence encoding a TCR beta chain or portion thereof carried or expressed by every lymphocyte present at a frequency of 0.0001 percent or greater. In some embodiments, clonotype profiles are derived from samples comprising from 103 to 107 lymphocytes. Such numbers of lymphocytes may be obtained from peripheral blood samples of from 1-10 mL.

“Coalescing” means treating two candidate clonotypes with sequence differences as the same by determining that such differences are due to experimental or measurement error and not due to genuine biological differences. In one aspect, a sequence of a higher frequency candidate clonotype is compared to that of a lower frequency candidate clonotype and if predetermined criteria are satisfied then the number of lower frequency candidate clonotypes is added to that of the higher frequency candidate clonotype and the lower frequency candidate clonotype is thereafter disregarded. That is, the read counts associated with the lower frequency candidate clonotype are added to those of the higher frequency candidate clonotype.

“Complementarity determining regions” (CDRs) means regions of an T cell receptor where the molecule complements an antigen's conformation, thereby determining the molecule's specificity and contact with a specific antigen. T cell receptors have three CDRs: CDR1 and CDR2 are encoded by the variable (V) genes, while CDR3 is encoded by the region between the variable (V) and joint (J) or diverse (D) and joint (J) genes and is highly variable.

“Epitope”/“neoepitope” refers to antigenic determinants, regions of proteins that can trigger a cellular immune response mediated by immune cells, such as T cells. T cell epitopes are usually protein antigen-derived peptides presented by MHC molecules on antigen-presenting cells and recognized by T cell receptors. Neoepitopes are those peptides that arise from somatic mutations, such as those that occur in cancer, and recognized as different from self and presented by antigen-presenting cells (APCs), such as, DCs and the tumor cells itself.

As used herein, the term “exogenous” refers to a molecule, nucleic acid, protein, or structure that is introduced into the cell by genetic or biochemical means. By contrast, an “endogenous” molecule, nucleic acid, protein, or structure is one that is present in the particular cell and/or in the particular cell at its developmental stage. An exogenous molecule, nucleic acid, protein, or structure can be the same type as an endogenous molecule, nucleic acid, protein, or structure found within the cell, or may be a type of molecule, nucleic acid, protein, or structure that is not normally found in the cell.

The term “exogenous TCR,” as used herein, refers to a recombinant TCR expressed in a cell via introduction of exogenous coding sequences for a TCR. Thus, the cell comprising an exogenous TCR is capable of expressing a TCR that is not natively expressed in that cell.

“Gene editing reagents” mean macromolecules designed to be used in methods of gene editing, and includes, but is not limited to, CRISPR/Cas9, cas-clover, MAD7 (cas12a/Cpf1), zinc finger nucleases (ZFNs), transcription activator-like (TAL) effector nucleases (TALENs).

“Human leukocyte antigen” or “HLA” refers to genes in major histocompatibility complexes (MHC) in humans that encode proteins that differentiate between self and non-self. They play a significant role in disease and immune defense. The HLA complex is synonymous with the human MHC. Human leukocyte antigens are of three main types. Class I HLA antigens include HLA-A, B, and C molecules; class II, which includes HLA-DR, -DQ, and -DP loci, are on antigen-presenting cells; and class III contains genes for proteins that have immune functionality. In the context of this invention, TCRs recognize antigens/neoantigens that are presented by a specific “self” HLA (HLA-A*11:01).

“Human T cell” refers to a T cell of any kind from a human that is an autologous (i.e., patient derived) or an allogeneic T cell. The T cell may, for example, be an allogeneic donor derived T cell or an iPSC-derived T cell.

The term “isolated,” as used herein, refers to a biological component such as a nucleic acid, peptide, protein, or cell that has been substantially separated, produced apart from, or purified away from other biological components of the organism in which the component naturally occurs. Nucleic acids, peptides, proteins, and cells that have been isolated thus include nucleic acids, peptides proteins, and cells that are purified by standard purification methods, or that are prepared by expression, for example expression in a host cell, or that are chemically synthesized. In some embodiments, the isolated cell is an autologous cell, meaning that it may is derived from the subject that will receive the resultant transduced or transformed cell. For example, in some embodiments, the isolated cells are derived from the PBMC and/or hematopoietic stem cells of the subject being treated.

“Knock in” means a type of genetic engineering in which an exogenous/heterologous gene construct is inserted into the genome of a cell. A gene “knock in” can be targeted or non-targeted. A targeted knock in means that the exogenous/heterologous gene construct is inserted at a defined location in the genome. A targeted knock in can be accomplished through a variety of gene manipulation techniques widely familiar to those of ordinary skill in the art, such as, by way of non-limiting example, through homology directed recombination (HDR), non-homologous end joining (NHEJ). Often, a gene editing reagent such as, by way of non-limiting examples, CRISPR-Cas9, cas-clover, MAD7, zinc fingers (ZFNs) or Transcription activator-like effector nucleases (TALENs), can be used to facilitate targeted knock in of an exogenous/heterologous gene construct. A non-targeted knock in means that the exogenous/heterologous gene construct is inserted a random/unspecified locus in the genome.

“Knock out” means a type of genetic engineering in which an endogenous gene or genes are deleted or suppressed. A gene “knock out” can be accomplished through various techniques, such as through homologous recombination or CRISPR-Cas9.

“Neoantigen”, as used herein, means a newly arising antigen to which the immune system is naïve. Neoantigens result from mutations in genes encoding endogenous proteins and arise during the development and progression of tumors/cancers. Targeting neoantigens provides a powerful new therapeutic modality to treat cancers and can be adapted to develop personalized cancer treatments by targeting the unique repertoire of neoantigens found in an individual patient's tumor(s).

“Non-virally inserted/delivered”, “non-viral insertion/delivery”, and the like, mean intracellular delivery of an exogenous/heterologous gene for the purpose of gene engineering/modification using delivery modalities that do not include the use of a viral or retroviral vector. Examples of non-viral insertion include, though are not limited to, electroporation (such as, for example, NUCLEOFECTOR® technology (Lonza, Basel, CH), or the NEON™ or XENON™ Electroporation Systems (Thermo Fisher Scientific, Waltham, MA), cationic lipids, chemical transfection.

“MHC” as used herein means a large locus on vertebrate DNA containing closely related polymorphic genes that encode molecules that bind peptide fragments derived from pathogens and display them on the cell surface for recognition by the appropriate T cells. Antigen presentation by major histocompatibility complex (MHC) proteins is essential for adaptive immunity. The MHC is located on chromosome 6 in humans and contains more than 200 genes. MHC genes are divided into three classes of MHC genes, the two major classes of which are MHC class I and MHC class II genes. MHC class I (pMHCI) complexes are presented on nucleated cells and are recognized by cytotoxic CD8+ T cells. The presentation of pMHCII by antigen-presenting cells (e.g., dendritic cells (DCs), macrophages, or B cells), on the other hand, can activate CD4+ T cells. In humans, MHC genes are called human leukocyte antigen (“HLA”) genes (i.e., “HLA I” and “MHC I” are synonymous in humans and used interchangeably).

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional

PCR using Taq DNA polymerase, a double stranded target nucleic acid may be denatured at a temperature>90° C., primers annealed at a temperature in the range 50-75° C., and primers extended at a temperature in the range 72-78° C.

The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL.

“Placeholder TCR” means a non-functional TCR alpha and/or TCR beta variable region that does not recognize any antigen/neoantigen and which is designed to be replaced by a therapeutically functional TCR alpha and/or TCR beta variable region that specifically binds a target antigen/neoantigen, such that after replacement of the nonfunctional variable region, cells expressing the TCR containing the functional variable regions can be administered as part of TCR cell therapy (TCT).

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references: Dieffenbach, editor, PCR Primer: A Laboratory Manual, 2nd Edition (Cold Spring Harbor Press, New York, 2003).

“Sequence read” means a sequence of nucleotides determined from a sequence or stream of data generated by a sequencing technique, which determination is made, for example, by means of base-calling software associated with the technique, e.g. base-calling software from a commercial provider of a DNA sequencing platform. A sequence read usually includes quality scores for each nucleotide in the sequence. Typically, sequence reads are made by extending a primer along a template nucleic acid, e.g. with a DNA polymerase or a DNA ligase. Data is generated by recording signals, such as optical, chemical (e.g. pH change), or electrical signals, associated with such extension. Such initial data is converted into a sequence read.

The term “subject” is herein defined as vertebrate, particularly mammal, more particularly human. For purposes of research, the subject may particularly be at least one animal model, e.g., a mouse, rat, pig, dog, non-human primate, and the like.

“T cell” as used herein refers to a diverse and important group of lymphocytes that mature and undergo a positive and negative selection processes in the thymus. These cells play a vital role in both components of active immunity, including cell-mediated and to some extent humoral immunity. There are several types of T cells; the most common and well-known are the CD4+ T cells (also known as helper T cells) and CD8+ T Cells (also known as cytotoxic T cells, cytolytic T cells, or killer T cells). T cells cannot recognize soluble, free antigens. T cells can only recognize protein-based, receptor-bound antigens. This recognition occurs via the use of the MHC (also known as HLA) I and II receptors, which along with the T cell receptors (TCRs) bind the antigen in question and form a complex that allows the T cell to recognize the antigen. CD4+ T cells recognize MHC II bound antigens, while CD8+ T cells recognize MHC I bound antigens. Both CD4+ T cells and CD8+ T cells have the TCR (and the co-receptor CD3), but (as evidenced by their name) their other co-receptors vary. CD4+ T cells have CD4, whereas CD8+ T cells have CD8 as an added co-receptor. a human T cell is autologous T cell, or allogeneic donor derived T cells, iPSC derived T cells etc, as stated above.

The term “therapeutically effective amount,” as used herein, refers to an amount that elicits an immune-mediated therapeutic effect in the subject. A therapeutic effect may include treatment of symptoms of a disease or disorder, or treatment of the underlying condition, and/or prophylaxis against development or worsening of a disease or disorder. In some embodiments, a “therapeutic vaccine” or “method of vaccination” and the like refers to a composition or method for eliciting an immune response against a pathogen or a component of a pathogen, such as to produce protective immunity (i.e., immunity that prevents or reduces severity of the disease associated with the pathogen).

### II. Antigen-Specific TCRs

In some embodiments, the present disclosure provides antigen/neoantigen-specific TCRs (e.g., antigen/neoantigen-specific TCR alpha and/or TCR beta chains). In some embodiments, such TCRs are specific for a G12D epitope/neoepitope on the KRAS oncoprotein.

### A. KRAS G12D

The Kirsten rat sarcoma viral oncogene homolog (KRAS) encodes a small GTPase that is a central component of the RAS/mitogen-activated protein kinase (RAS/MAPK) signal transduction pathway. This pathway is composed of an intracellular network of proteins that transmit extracellular growth factor signals to regulate cell proliferation, differentiation, and survival. KRAS is the most frequently mutated oncogene with mutations found in approximately 14% of all cancers. Mutations in the KRAS gene can result in alterations at several amino acid positions, including glycine 12 (G12), glycine 13, and glutamine 61, which are commonly found in solid tumors and associated with tumorigenesis and aggressive tumor growth. Because the peptides derived from these cancer-specific alterations can be presented by HLA receptors on the surface of cancer cells, they are attractive as mutant cancer neoantigen targets for T cell therapies with applicability in a number of indications. Specifically, oncogenic KRAS mutations that result in the change from G12 to aspartic acid (G12D) are prevalent in pancreatic ductal adenocarcinoma (PDAC) (40% of tumors), colorectal cancer (CRC) (15% of tumors), non-small cell lung cancer (NSCLC) (5% of tumors) as well as in other tumor types. Advanced stage tumors harboring the KRAS G12D mutation (hereafter referred to as KRAS G12D-positive cancers), include PDAC, CRC, NSCLC, and other solid tumors that carry a poor prognosis.

### B. TCR Cell Therapy (TCT)

TCR cell therapy is an autologous or allogeneic (such as an donor or iPSC-derived) T cell therapy containing CD8+ T cells engineered through non-viral delivery gene editing reagents to express the α/βT cell receptor EE209_2 (also referred to as EE209_KRAS_2) that is specific for the KRAS G12D-derived peptide VVVGADGVGK (SEQ ID NO: 1) in the context of HLA-A*11:01 presentation. The EE209_2 TCR comprises a TCR-alpha chain comprising a TCR-alpha variable region and a TCR-beta chain comprising a TCR-beta variable region, wherein the TCR-alpha variable region comprises a CDR1 comprising the amino acid sequence SEQ ID NO: 7, a CDR2 comprising the amino acid sequence SEQ ID NO: 8, and a CDR3 comprising amino the acid sequence SEQ ID NO: 9, and wherein the TCR-beta variable region comprises a CDR1 comprising the amino acid sequence SEQ ID NO: 3, a CDR2 comprising the amino acid sequence SEQ ID NO: 4, and a CDR3 comprising the amino acid sequence SEQ ID NO: 6. In some embodiments, the TCR-alpha chain variable region of the EE209_2 TCR comprises SEQ ID NO: 10 and the TCR-beta chain variable region of the EE209_2 TCR comprises SEQ ID NO: 11. In some embodiments, the TCR-alpha chain of the EE209_2 TCR comprises SEQ ID NO: 12 and the TCR-beta chain of the EE209_2 TCR comprises SEQ ID NO: 13, 14, or 15, which are TRBC2-201, TRBC1-201, and TRBC2-202, respectively. TRBC2-201, TRBC1-201, and TRBC2-202 represent three different isoforms of the TCR beta constant region (TRBC) gene.

In some embodiments, a manufactured engineered T cell product for a patient meets a minimum total cell dose requirement (at least 7.5×10⁸ total cells). The EE209_2 TCR is a TCR that was selected through a process involving peptide stimulation of peripheral T cells from a healthy donor, identification of KRAS G12D-specific TCR beta chains, matching of cognate alpha and beta chains, and extensive functional assessments. The EE209_2 TCR is integrated into the TRAC region and is expressed under control of the endogenous TCR promoter. Expression of endogenous TCR a and B chains is disrupted by CRISPR/Cas9-mediated gene knockout, which avoids mispairing of transgenic and endogenous TCR α and β chains.

Nonclinical studies have demonstrated that CD8⁺ T cells expressing the EE209_2 TCR upregulate the activation marker CD137 and mediate specific target cell lysis in response to T2 cells loaded with peptide VVVGADGVGK (SEQ ID NO: 1), as well as various tumor cell lines expressing KRAS G12D and HLA-A*11:01.

In some embodiments of the present invention, the KRAS G12-derived neoantigen to which the recombinant TCR binds comprises the 10-mer peptide VVVGADGVGK (SEQ ID NO: 1). In some embodiments, the KRAS G12-derived neoantigen to which the recombinant TCR binds comprises the 9-mer peptide VVGADGVGK (SEQ ID NO: 2). In some embodiments, the TCR binds the KRAS neoantigen in a subject who is HLA-A*11:01 positive. In some embodiments, the TCR activates a T cell upon binding to the KRAS G12D neoantigen.

### III. Cell Engineering

The invention provides engineered cells that express the recombinant TCR that specifically binds to the KRAS G12D neoantigen. As described below, the engineered cells may be of any of several cell types and the recombinant TCR may be introduced by any of several methods.

### A. Cell Types

A variety of cell types expressing the recombinant TCR may be used in the embodiments of the invention. In some embodiments, the engineered cell are T cells. In some embodiments, the engineered cells are autologous (i.e., patient derived) T cells. In some embodiments, the engineered cells are allogeneic T cells. In some embodiments, the engineered cells are allogeneic donor-derived T cells. In some embodiments, the donor-derived T cells are derived from healthy donors having the same or different HLA haplotype as a patient to whom the engineered T cells will ultimately be administered.

In some embodiments, the engineered cells are autologous or allogeneic iPSC cells. In some embodiments, the engineered cells are autologous or allogeneic iPSC-derived T cells. In some embodiments, the T cells are CD8+ T cells. In some embodiments, the iPSC cells or T cells are stored in a cell bank for future use or further editing.

In some embodiments, the recombinant TCRs are “knocked in” to T cells, which may in some embodiments be autologous T cells and may in other embodiments be allogeneic T cells. In some embodiments, the recombinant TCRs may be knocked in to iPSCs, which are subsequently differentiated into T cells. In some embodiments, allogeneic T cells are engineered with recombinant TCRs, which may be a “placeholder TCR variable region” or the recombinant TCR of this invention. In the recombinant TCR is a “placeholder TCR,” the alpha and beta variable regions is subsequently replaced by the KRAS G12D neoantigen-specific recombinant TCR of the invention.

### B. Methods of Cell Engineering

The present invention provides methods of producing the engineered cells comprising the recombinant TCRs. The recombinant TCRs of the invention may be transfected or introduced to host cells through various methods of cell engineering. In some embodiments, the resulting host cells, such as T cells, may express the recombinant TCRs. In some embodiments, host cells expressing the recombinant TCRs are used in methods of adoptive immunotherapy.

The recombinant TCRs may be introduced to host cells, such as T cells, through the methods of editing the genome of a cell, for example, as taught in Oh, et al, J Exp Med. 2022 May 2; 219(5) and in PCT Publ. No. WO2022/204443A1.

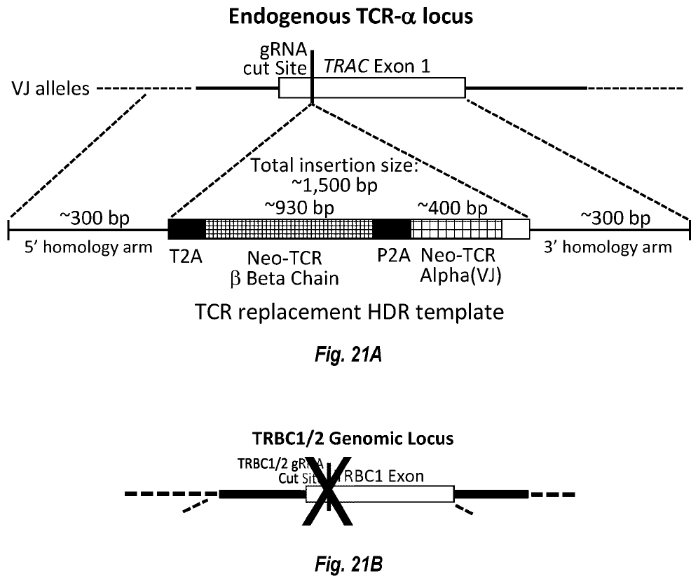

In some embodiments, the T cell engineering method involves knock-in of the recombinant TCR into the endogenous TCR-alpha chain (TRAC) locus as shown in FIG. 21A. In some embodiments, the construct to be knocked in comprises a nucleotide sequence encoding, from N-terminus to C-terminus, a T2A self-cleaving peptide, a heterologous TCR beta chain, a P2A self-cleaving peptide, and a heterologous TCR alpha variable region, and a portion of the N-terminus of the endogenous TCR alpha constant region. In some embodiments, the T cell engineering method involves knock-out of the endogenous TCR beta locus as shown in FIG. 21B. In some embodiments, both copies of the endogenous TCR beta locus are knocked out. In some embodiments, only one copy of the endogenous TCR beta locus is knocked out. In some embodiments, the T cell engineering method involves non-viral mediated transfer of a nucleic acid encoding the recombinant TCR into the TCR-alpha chain (TRAC) locus of the genome. In some embodiments, the insertion comprises homologous recombination within the TRAC locus, optionally within the TCR-alpha constant region. In some embodiments, the nucleic acid encodes the desired TCR-beta chain and part of the TCR-alpha chain (the N terminal portion that encodes the desired TCR-alpha). In some embodiments, the 3′-flanking arm of the nucleic acid is homologous with the TCR-alpha constant region such that it recombines with the endogenous genomic TCR-alpha constant region. In some embodiments, the method of engineering the T cell further comprises knocking out both copies of the endogenous TCR-beta gene to prevent any alpha/beta chain mispairing in the engineered cells. In some embodiments, the T cell engineering method comprises using CRISPR/Cas9 and sgRNAs to target exon 1 of the endogenous TRAC and TCR-beta chain (TRBC) loci to knock out the endogenous TCR. In some embodiments, the T cell engineering method involves homology-directed repair using a construct containing sequences encoding the desired TCR-beta chain and TCR-alpha chain.

In some embodiments, the genomes of host cells are edited through non-viral T cell engineering methods as taught by Roth, et al, WO2018/232356 and WO2019/084552, U.S. Pat. Nos. 11,033,584, 11,083,753, and 11,331,346. In some embodiments, the recombinant TCR is introduced by a method of editing the genome of a T cell, the method comprising inserting into a target region in exon 1 of a T cell receptor (TCR)-subunit constant gene in the human T cell a nucleic acid sequence encoding, from the N-terminus to the C-terminus, (i) a first self-cleaving peptide sequence; (ii) a first heterologous TCR subunit chain, wherein the TCR subunit chain comprises the variable region and the constant region of the TCR subunit; (iii) a second self-cleaving peptide sequence; (iv) a variable region of a second heterologous TCR subunit chain; and (v) a portion of the N-terminus of the endogenous TCR subunit, wherein, if the endogenous TCR subunit is a TCR-alpha (TCR-a) subunit, the first heterologous TCR subunit chain is a heterologous TCR-beta (TCR-β) subunit chain and the second heterologous TCR subunit chain is a heterologous TCR-a subunit chain, and wherein if the endogenous TCR subunit is a TCR-β subunit, the first heterologous TCR subunit chain is a heterologous TCR-a subunit chain and the second heterologous TCR subunit chain is a heterologous TCR-β subunit chain. In some embodiments of the method of editing the genome, a nucleic acid sequence encoding, from the N-terminus to the C-terminus, (i) a first self-cleaving peptide sequence; (ii) a heterologous TCR-β chain; (iii) a second self-cleaving peptide sequence; (iv) a variable region of a heterologous TCR-α chain; and (v) a portion of the N-terminus of the endogenous TCR-a subunit, is inserted into exon 1 of a TCR-alpha subunit constant gene (TRAC) in the human T cell.

In some embodiments, the recombinant TCR is introduced by a method of editing the genome of a cell, the method comprising a gene editing reagent wherein the gene editing reagent or components thereof specifically hybridize to a target genome of the genome of the cell, a nuclease cleaves the target region to create an insertion site in the genome of the cell, and a double-stranded or single-stranded DNA template comprising sequences homologous to genomic sequences flanking the insertion site are introduced to the cell. In some embodiments, the recombinant TCR is introduced by a method of editing the genome of a cell, the method comprising: a) providing a Cas9 ribonucleoprotein complex (RNP)-DNA template complex comprising: (i) the RNP, wherein the RNP comprises a Cas9 nuclease domain and a guide RNA, wherein the guide RNA specifically hybridizes to a target region of the genome of the cell, and wherein the Cas9 nuclease domain cleaves the target region to create an insertion site in the genome of the cell; and (ii) a double-stranded or single-stranded DNA template, wherein the size of the DNA template is greater than about 200 nucleotides, wherein the 5′ and 3′ ends of the DNA template comprise nucleotide sequences that are homologous to genomic sequences flanking the insertion site, and wherein the molar ratio of RNP to DNA template in the complex is from about 3:1 to about 100:1; and b) introducing the RNP-DNA template complex into the cell.

In some embodiments, the engineered T cell is a human T cell human T cell comprising: at least one nucleic acid sequence comprising at least one heterologous gene non-virally inserted into one or both of: an endogenous T cell receptor alpha subunit constant gene (TRAC), and an endogenous T cell receptor beta subunit constant gene (TRBC), wherein the at least one heterologous gene comprises at least one of: (1) a variable region of a heterologous human T cell receptor alpha (TCR-α) chain gene and (2) a variable region of a heterologous human T cell receptor beta (TCR-β) chain gene. In some embodiments, the heterologous gene comprises (1) a TCR alpha variable region encoding SEQ ID NO: 10 and (2) a TCR beta variable region encoding SEQ ID NO: 11. In some embodiments, the at least one heterologous gene comprises (1) a portion of a TCR alpha region encoding SEQ ID NO: 12 and (2) a TCR beta region encoding SEQ ID NO: 13, 14, or 15. In some embodiments, the engineered T cell is a human T cell comprising: at least one nucleic acid sequence comprising at least one heterologous gene inserted into one or both of: an endogenous T cell receptor alpha subunit constant gene (TRAC), and an endogenous T cell receptor beta subunit constant gene (TRBC), wherein the at least one heterologous gene comprises at least one of: (1) a variable region of a heterologous human T cell receptor alpha (TCR-α) chain gene and (2) a variable region of a heterologous human T cell receptor beta (TCR-β) chain gene, wherein the T cell does not comprise a viral vector for introducing the at least one nucleic acid sequence to the T cell. In some embodiments, the heterologous gene comprises (1) a TCR alpha variable region encoding SEQ ID NO: 10 and (2) a TCR beta variable region encoding SEQ ID NO: 11. In some embodiments, the at least one heterologous gene comprises (1) a portion of a TCR alpha region encoding SEQ ID NO: 12 and (2) a TCR beta region encoding SEQ ID NO: 13, 14, or 15. In some embodiments, the engineered T cell does not comprise any exogenously introduced viral sequences.

In some embodiments, the recombinant TCR is introduced to the host cell using methods of nuclease-mediated gene editing as taught by Jacoby, et al, U.S. Pat. No. 10,550,406. In some embodiments, the method comprises modifying a patient-derived T cell by a nuclease-mediated introduction of a non-viral polynucleotide into the T cell, wherein the non-viral polynucleotide comprises: i. first and second homology arms homologous to first and second endogenous sequences of the cell; ii. a TCR gene sequence positioned between the first and second homology arms; iii. a first P2A-coding sequence positioned upstream of the TCR gene sequence and a second P2A-coding sequence positioned downstream of the TCR gene sequence, wherein the first and second P2A-coding sequences code for the same amino acid sequence that are codon-diverged relative to each other; iv. a sequence coding for the amino acid sequence Gly Ser Gly positioned immediately upstream of the P2A-coding sequences; and v. a sequence coding for a Furin cleavage site positioned upstream of the second P2A-coding sequence; b. recombining the non-viral polynucleotide into an endogenous locus of the T cell, wherein the endogenous locus comprises the first and second endogenous sequences homologous to the first and second homology arms of the non-viral polynucleotide; c. culturing the modified T cell to produce a population of T cells; and d. administering a therapeutically effective number of the modified T cells to the human patient to thereby treat the cancer. P2A refers to a member of the 2A self-cleaving peptide family. In some embodiments, the method further comprises recombination of the non-viral polynucleotide into the endogenous locus by homology directed repair.

In some embodiments, T cells are engineered using a method for efficient TCR gene editing as taught in WO2022/204443A1 Such methods address the low efficiency and low numbers of engineered T cells that are generally produced using known non-viral methods. In some embodiments, the method for making an engineered T cell comprises contacting a T cell with a first ribonucleoprotein particle (RNP) and a donor DNA, wherein the first RNP comprises a first guide RNA that targets an endogenous TCR-locus, and wherein the donor DNA comprises a nucleic acid sequence comprising a gene encoding a polypeptide comprising an exogenous TCR-beta and an exogenous TCR-alpha or portion thereof, under conditions to allow the RNP and the donor DNA to enter the cell, incubating the T cell for a period of time, and culturing the cell in a medium for a period of time to allow the donor DNA to be inserted into the endogenous TCR-alpha locus, thereby forming an engineered cell. In some embodiments, the exogenous TCR-alpha comprise full-length TCR-alpha. In some embodiments, the exogenous TCR-alpha or portion thereof comprises the TCR-alpha (VJ) domain. In some embodiments, the TCR locus is a TCR-alpha locus, and the T cell is contacted with a second RNP comprising a second guide RNA that targets an endogenous TCR-beta locus.

In some embodiments, a nucleic acid sequence encoding one or more subunits of the TCR of the invention is introduced to host cells using viral transduction systems, such as an adenoviral, retroviral, or lentiviral vector. In some embodiments, the nucleic acid sequence encoding one or more subunits of the TCR of the invention is introduced to host cells using transposase-based genome engineering, which can either comprise a retrotransposon or a “cut and paste” transposable element. In some embodiments, the TCR is introduced to host cells via targeted replacement of the TCR alpha locus, the TCR beta locus, or both with a nucleic acid sequence encoding one or more subunits of the TCR. In some embodiments, the TCR is introduced to host cells via targeted replacement of a non-TCR alpha or TCR-beta locus with a nucleic acid sequence encoding one or more subunits of the TCR. In some embodiments, the TCR is introduced to host cells via non-targeted replacement methods. In some embodiments in which the TCR is introduced to host cells at a locus outside the endogenous TCR-alpha or TCR-beta locus, both the endogenous TCR-alpha and endogenous TCR-beta genes must be knocked out.

### IV. Amplification of Nucleic Acid Populations

In some embodiments, nucleic acids encoding immune cell receptors specific to KRAS G12D are sequenced, a process which includes nucleic acid extraction, amplification, and sequencing. In some embodiments, this process is used to identify nucleic acids encoding KRAS G12D specific TCRs during TCR discovery.

In some embodiments, amplicons of target populations of nucleic acids may be generated by a variety of amplification techniques. In one aspect of the invention, multiplex PCR is used to amplify members of a mixture of nucleic acids, particularly mixtures comprising recombined immune molecules such as T cell receptors, or portions thereof. Guidance for carrying out multiplex PCRs of such immune molecules is found in the literature, including the following references, which are incorporated by reference: U.S. Pat. Nos. 8,236,503; 8,628,927; 5,296,351; 5,837,447; 6,087,096; 8,859,748; European Patent EP1544308.

After amplification of DNA from the genome (or amplification of nucleic acid in the form of cDNA by reverse transcribing RNA), the individual nucleic acid molecules can be isolated, optionally re-amplified, and then sequenced individually. Exemplary amplification protocols may be found in van Dongen et al, Leukemia, 17: 2257-2317 (2003) or van Dongen et al, US Patent Application Publication No. 2006/0234234. Briefly, an exemplary protocol is as follows: Reaction buffer: ABI Buffer II or ABI Gold Buffer (Life Technologies, San Diego, Calif.); 50 μL final reaction volume; 100 ng sample DNA; 10 μmol of each primer (subject to adjustments to balance amplification as described below); dNTPs at 200 μM final concentration; MgCl₂ at 1.5 mM final concentration (subject to optimization depending on target sequences and polymerase); Taq polymerase (1-2 U/tube); cycling conditions: pre-activation 7 min at 95° C.; annealing at 60° C.; cycling times: 30 s denaturation; 30 s annealing; 30s extension. Polymerases that can be used for amplification in the methods of the invention are commercially available and include, for example, Taq polymerase, AccuPrime polymerase, or Pfu. The choice of polymerase to use can be based on whether fidelity or efficiency is preferred.

Real time PCR, picogreen staining, nanofluidic electrophoresis (e.g. LabChip) or UV absorption measurements can be used in an initial step to judge the functional amount of amplifiable material.

In one aspect, multiplex amplifications are carried out so that relative amounts of sequences in a starting population are substantially the same as those in the amplified population, or amplicon. That is, multiplex amplifications are carried out with minimal amplification bias among member sequences of a sample population. In one embodiment, such relative amounts are substantially the same if each relative amount in an amplicon is within five fold of its value in the starting sample. In another embodiment, such relative amounts are substantially the same if each relative amount in an amplicon is within two fold of its value in the starting sample. As discussed more fully below, amplification bias in PCR may be detected and corrected using conventional techniques so that a set of PCR primers may be selected for a predetermined repertoire that provide unbiased amplification of any sample.

In one embodiment, amplification bias may be avoided by carrying out a two-stage amplification (as described in U.S. Pat. No. 8,691,510 wherein a small number of amplification cycles are implemented in a first, or primary, stage using primers having tails non-complementary with the target sequences. The tails include primer binding sites that are added to the ends of the sequences of the primary amplicon so that such sites are used in a second stage amplification using only a single forward primer and a single reverse primer, thereby eliminating a primary cause of amplification bias. Preferably, the primary PCR will have a small enough number of cycles (e.g. 5-10) to minimize the differential amplification by the different primers. The secondary amplification is done with one pair of primers and hence the issue of differential amplification is minimal. One percent of the primary PCR is taken directly to the secondary PCR. Thirty-five cycles (equivalent to ~28 cycles without the 100 fold dilution step) used between the two amplifications were sufficient to show a robust amplification irrespective of whether the breakdown of cycles were: one cycle primary and 34 secondary or 25 primary and 10 secondary. Even though ideally doing only 1 cycle in the primary PCR may decrease the amplification bias, there are other considerations. One aspect of this is representation. This plays a role when the starting input amount is not in excess to the number of reads ultimately obtained. For example, if 1,000,000 reads are obtained and starting with 1,000,000 input molecules then taking only representation from 100,000 molecules to the secondary amplification would degrade the precision of estimating the relative abundance of the different species in the original sample. The 100 fold dilution between the 2 steps means that the representation is reduced unless the primary PCR amplification generated significantly more than 100 molecules. This indicates that a minimum 8 cycles (256 fold), but more comfortably 10 cycle (~1,000 fold), may be used. The alternative to that is to take more than 1% of the primary PCR into the secondary but because of the high concentration of primer used in the primary PCR, a big dilution factor can be used to ensure these primers do not interfere in the amplification and worsen the amplification bias between sequences. Another alternative is to add a purification or enzymatic step to eliminate the primers from the primary PCR to allow a smaller dilution of it. In this example, the primary PCR was 10 cycles and the second 25 cycles.

### V. Generating Sequence Reads for Clonotypes

Any high-throughput technique for sequencing nucleic acids can be used in the methods of the invention. Preferably, such technique has a capability of generating in a cost-effective manner a volume of sequence data from which at least 1000 clonotypes can be determined, and preferably, from which at least 10,000 to 1,000,000 clonotypes can be determined. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLID sequencing. Sequencing of the separated molecules has been carried out by sequential or single extension reactions using polymerases or ligases as well as by single or sequential differential hybridizations with libraries of probes. These reactions have been performed on many clonal sequences in parallel including demonstrations in current commercial applications of over 100 million sequences in parallel. These sequencing approaches can thus be used to study the repertoire of T cell receptors (TCRs).

In one aspect of the invention, high-throughput methods of sequencing are employed that comprise a step of spatially isolating individual molecules on a solid surface where they are sequenced in parallel. Such solid surfaces may include nonporous surfaces (such as in “Solexa sequencing”, e.g. Bentley et al, Nature, 456:53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, US patent publication 2010/0137143 or 2010/0304982), micromachined membranes (such as with SMRT sequencing, e.g. Eid et al, Science, 323: 133-138 (2009)), or bead arrays (as with SOLID sequencing or polony sequencing, e.g. Kim et al, Science, 316: 1481-1414 (2007)).

In another aspect, such methods comprise amplifying the isolated molecules either before or after they are spatially isolated on a solid surface. Prior amplification may comprise emulsion-based amplification, such as emulsion PCR, or rolling circle amplification. Of particular interest is Solexa-based sequencing where individual template molecules are spatially isolated on a solid surface, after which they are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010); and further in the following references: U.S. Pat. Nos. 6,090,592; 6,300,070; 7,115,400; and EP0972081. In one embodiment, individual molecules disposed and amplified on a solid surface form clusters in a density of at least 105 clusters per cm2; or in a density of at least 5×10⁵ per cm2; or in a density of at least 106 clusters per cm2. In one embodiment, sequencing chemistries are employed having relatively high error rates. In such embodiments, the average quality scores produced by such chemistries are monotonically declining functions of sequence read lengths.

In one aspect, a sequence-based clonotype profile of an individual is obtained using the following steps: (a) obtaining a nucleic acid sample, for example, a sample containing T cells of the individual; (b) spatially isolating individual molecules derived from such nucleic acid sample, the individual molecules comprising at least one template generated from a nucleic acid in the sample, which template comprises a somatically rearranged region or a portion thereof, each individual molecule being capable of producing at least one sequence read; (c) sequencing said spatially isolated individual molecules to provide sequence reads; and (d) determining abundances of different sequences of the nucleic acid molecules from the nucleic acid sample to generate the clonotype profile. In some embodiments, the step of sequencing includes coalescing at least a plurality of sequence reads to form each clonotype. As described more fully below, such a step of coalescing is a process of combining sequence reads with error rates (for example, from sequencing and/or amplification errors) to produce clonotypes that are correct with a high degree of likelihood, such as with a 99% confidence level.

In one aspect, for each sample from an individual, the sequencing technique used in the methods of the invention generates sequences of least 1000 sequence reads per run; in another aspect, such technique generates sequences of at least 10,000 sequence reads per run; in another aspect, such technique generates sequences of at least 100,000 sequence reads per run; in another aspect, such technique generates sequences of at least 500,000 sequence reads per run; and in another aspect, such technique generates sequences of at least 1,000,000 sequence reads per run. From such sequence reads clonotypes are determined, for example, as described below, or as disclosed in U.S. Pat. No. 8,691,510 (described above).

The sequencing techniques used in the methods generate sequence reads having lengths of at least 30 nucleotides. In some embodiments, a step of sequencing generates sequence reads having lengths of at least 50 nucleotides; and in some embodiments, a step of sequencing generates sequence reads having lengths of at least 100 nucleotides.

A. Clonotype Determination from Sequence Data

In another aspect of the invention, sequences of clonotypes are determined in part by aligning sequence reads to one or more V region reference sequences and one or more J region reference sequences, and in part by base determination without alignment to reference sequences, such as in the highly variable NDN region. A variety of alignment algorithms may be applied to the sequence reads and reference sequences. For example, guidance for selecting alignment methods is available in Batzoglou, Briefings in Bioinformatics, 6: 6-22 (2005). In one aspect, whenever V reads (as mentioned above) are aligned to V and J region reference sequences, a tree search algorithm may be employed, e.g. as described generally in Gusfield (cited above) and Cormen et al, Introduction to Algorithms, Third Edition (The MIT Press, 2009).

### VI. Methods of Determining Antigen-Specific T Cell Receptors

In some embodiments, the present disclosure provides methods of determining KRAS G12D neoantigen-specific T cell receptors from a sample containing T cells.

In some embodiments, methods for determining KRAS G12D neoantigen-specific TCRs from a sample containing T cells are used as taught by Klinger et al, U.S. Pat. No. 10,066,265, which is incorporated by reference. In some embodiments, the method used for determining KRAS G12D neoantigen-specific TCRs from a sample containing T cells is Multiplex Identification of Antigen-Specific T Cell Receptors Using a Combination of Immune Assays and Immune Receptor Sequencing or MIRA, as taught by in Klinger, et al, PLOS One. 2015 Oct. 28; 10(10). In some embodiments, methods of determining antigen-specific T cell receptors from a sample containing T cells comprise the following steps: (a) sequencing recombined nucleic acids encoding one or more TCR chain(s), or a portion thereof, from a first portion of the sample to generate a first multiplicity (number greater than 2) of sequence reads obtained from unstimulated T cells; (b) partitioning a second portion of the sample into a plurality (number equal or greater than 2) of reaction mixtures and exposing each reaction mixture of the plurality of reaction mixtures to antigens; (c) for each reaction mixture in the plurality of reaction mixtures, separating T cells that interact with one or more antigens in the reaction mixture to obtain a subset of antigen-specific T cells, wherein each of the subsets of antigen-specific T cells corresponds to one reaction mixture in the plurality of reaction mixtures; (d) for each of the subsets of antigen-specific T cells separated in step (c), sequencing recombined nucleic acids encoding one or more TCR chain(s), or a portion thereof, to generate sequence reads obtained from each of the subsets of antigen-specific T cells; (e) for each reaction mixture in the plurality of reaction mixtures, identifying a plurality of antigen-specific TCR chains, or portion thereof, by comparing the sequence reads obtained from each of the subsets of antigen-specific T cells in step (d) to the sequence reads obtained from unstimulated T cells in step (a), wherein the frequency of the sequence reads for the KRAS G12D neoantigen-specific TCR chains, or portion thereof, is increased in the sequence reads obtained from the subsets of antigen-specific T cells compared to the frequency of sequence reads for the antigen-specific TCR chains, or portion thereof, in the sequence reads obtained from unstimulated T cells; and (f) identifying one or more TCR chains, or portion thereof, specific for one of the antigens from the one or more TCR chains, or a portion thereof, identified in step (e), wherein the frequency of the sequence reads for the one or more TCR chains, or portion thereof, specific for the antigen is increased in the sequence reads obtained from each of the subsets of antigen-specific T cells in which the antigen was present in the corresponding reaction mixture.

In some embodiments, the present disclosure provides a T cell receptor (TCR) that binds to a KRAS G12D neoantigen comprising a TCR-alpha chain and a TCR-beta chain, the TCR-alpha variable region comprises a CDR1 comprising the amino acid sequence SEQ ID NO: 7, a CDR2 comprising the amino acid sequence SEQ ID NO: 8, and a CDR3 comprising amino the acid sequence SEQ ID NO: 9; and wherein the TCR-beta variable region comprises the following: a CDR1 comprising the amino acid sequence SEQ ID NO: 3, a CDR2 comprising the amino acid sequence SEQ ID NO: 4, and a CDR3 comprising the amino acid sequence SEQ ID NO: 6, and wherein the antigen specificity of the TCR or portion thereof is determined by a method comprising the steps of: (a) sequencing recombined nucleic acids encoding one or more TCR chain(s), or a portion thereof, from a first portion of a sample comprising T cells to generate a first multiplicity of sequence reads obtained from T cells prior to KRAS G12D neoantigen exposure of the sample, wherein the sequencing is high-throughput sequencing; (b) partitioning a second portion of the sample comprising T cells into a plurality of reaction mixtures and exposing each reaction mixture of the plurality of reaction mixtures to KRAS G12D neoantigens; (c) for each reaction mixture in the plurality of reaction mixtures, separating T cells that interact with one or more KRAS G12D neoantigens in the reaction mixture to obtain a subset of KRAS G12D neoantigen-specific T cells, wherein each of the subsets of antigen-specific T cells corresponds to one reaction mixture in the plurality of reaction mixtures; (d) for each of the subsets of KRAS G12D neoantigen-specific T cells separated in step (c), sequencing recombined nucleic acids encoding one or more TCR chain(s), or a portion thereof, to generate a multiplicity of sequence reads obtained from each of the subsets of antigen-specific T cells, wherein the sequencing is high-throughput sequencing; (e) for each reaction mixture in the plurality of reaction mixtures, identifying a plurality of antigen-specific TCR chains, or portion thereof, by comparing the multiplicity of sequence reads obtained from each of the subsets of KRAS G12D neoantigen-specific T cells in step (d) to the first multiplicity of sequence reads obtained from unstimulated T cells in step (a), wherein the frequency of the sequence reads for the KRAS G12D neoantigen-specific TCR chains, or portion thereof, is increased in the multiplicity of sequence reads obtained from the subsets of antigen-specific T cells compared to the frequency of sequence reads for the antigen-specific TCR chains, or portion thereof, in the first multiplicity of sequence reads obtained from unstimulated T cells; and (f) identifying one or more TCR chains, or portion thereof, specific for the KRAS G12D neoantigen from the one or more TCR chains, or a portion thereof, identified in step (e), wherein the frequency of the sequence reads for the one or more TCR chains, or portion thereof, specific for the antigen is increased in the multiplicity of sequence reads obtained from each of the subsets of antigen-specific T cells in which the antigen was present in the corresponding reaction mixture.

In some embodiments, the present disclosure provides a T cell receptor (TCR) that binds to a KRAS G12D neoantigen comprising a TCR-alpha chain and a TCR-beta chain, the TCR-alpha variable region comprises a TCR-alpha chain variable region and a TCR-beta chain variable region wherein the TCR comprises (a) a TCR-alpha chain variable region comprising a CDR1, a CDR2, and a CDR3 comprising amino acid sequences SEQ ID NOs: 19, 20, and 21, respectively, and a TCR-beta chain variable region comprising a CDR1, a CDR2, and a CDR3 comprising amino acid sequences SEQ ID NOs: 16, 17, and 18, respectively; or (b) a TCR-alpha chain variable region comprising a CDR1, a CDR2, and a CDR3 comprising amino acid sequences SEQ ID NOs: 29, 30, and 31, respectively, and a TCR-beta chain variable region comprising a CDR1, a CDR2, and a CDR3 comprising amino acid sequences SEQ ID NOs: 26, 27, and 28, respectively; or (c) a TCR-alpha chain variable region comprising a CDR1, a CDR2, and a CDR3 comprising amino acid sequences SEQ ID NOs: 39, 40, and 41, respectively, and a TCR-beta chain variable region comprising a CDR1, a CDR2, and a CDR3 comprising amino acid sequences SEQ ID NOs: 36, 37, and 38, respectively; or (d) a TCR-alpha chain variable region comprising a CDR1, a CDR2, and a CDR3 comprising amino acid sequences SEQ ID NOs: 49, 50, and 51, respectively, and a TCR-beta chain variable region comprising a CDR1, a CDR2, and a CDR3 comprising amino acid sequences SEQ ID NOs: 46, 47, and 48, respectively; or (e) a TCR-alpha chain variable region comprising a CDR1, a CDR2, and a CDR3 comprising amino acid sequences SEQ ID NOs: 59, 60, and 61, respectively, and a TCR-beta chain variable region comprising a CDR1, a CDR2, and a CDR3 comprising amino acid sequences SEQ ID NOs: 56, 57, and 58, respectively; or (f) a TCR-alpha chain variable region comprising a CDR1, a CDR2, and a CDR3 comprising amino acid sequences SEQ ID NOs: 69, 70, and 71, respectively, and a TCR-beta chain variable region comprising a CDR1, a CDR2, and a CDR3 comprising amino acid sequences SEQ ID NOs: 66, 67, and 68, respectively; or (g) a TCR-alpha chain variable region comprising a CDR1, a CDR2, and a CDR3 comprising amino acid sequences SEQ ID NOs: 79, 80, and 81, respectively, and a TCR-beta chain variable region comprising a CDR1, a CDR2, and a CDR3 comprising amino acid sequences SEQ ID NOs: 76, 77, and 78, respectively; or (h) a TCR-alpha chain variable region comprising a CDR1, a CDR2, and a CDR3 comprising amino acid sequences SEQ ID NOs: 89, 90, and 91, respectively, and a TCR-beta chain variable region comprising a CDR1, a CDR2, and a CDR3 comprising amino acid sequences SEQ ID NOs: 86, 87, and 88, respectively, wherein the antigen specificity of the TCR or portion thereof is determined by a method comprising the steps of: (a) sequencing recombined nucleic acids encoding one or more TCR chain(s), or a portion thereof, from a first portion of a sample comprising T cells to generate a first multiplicity of sequence reads obtained from T cells prior to KRAS G12D neoantigen exposure of the sample, wherein the sequencing is high-throughput sequencing; (b) partitioning a second portion of the sample comprising T cells into a plurality of reaction mixtures and exposing each reaction mixture of the plurality of reaction mixtures to KRAS G12D neoantigens; (c) for each reaction mixture in the plurality of reaction mixtures, separating T cells that interact with one or more KRAS G12D neoantigens in the reaction mixture to obtain a subset of KRAS G12D neoantigen-specific T cells, wherein each of the subsets of antigen-specific T cells corresponds to one reaction mixture in the plurality of reaction mixtures; (d) for each of the subsets of KRAS G12D neoantigen-specific T cells separated in step (c), sequencing recombined nucleic acids encoding one or more TCR chain(s), or a portion thereof, to generate a multiplicity of sequence reads obtained from each of the subsets of antigen-specific T cells, wherein the sequencing is high-throughput sequencing; (e) for each reaction mixture in the plurality of reaction mixtures, identifying a plurality of antigen-specific TCR chains, or portion thereof, by comparing the multiplicity of sequence reads obtained from each of the subsets of KRAS G12D neoantigen-specific T cells in step (d) to the first multiplicity of sequence reads obtained from unstimulated T cells in step (a), wherein the frequency of the sequence reads for the KRAS G12D neoantigen-specific TCR chains, or portion thereof, is increased in the multiplicity of sequence reads obtained from the subsets of antigen-specific T cells compared to the frequency of sequence reads for the antigen-specific TCR chains, or portion thereof, in the first multiplicity of sequence reads obtained from unstimulated T cells; and (f) identifying one or more TCR chains, or portion thereof, specific for the KRAS G12D neoantigen from the one or more TCR chains, or a portion thereof, identified in step (e), wherein the frequency of the sequence reads for the one or more TCR chains, or portion thereof, specific for the antigen is increased in the multiplicity of sequence reads obtained from each of the subsets of antigen-specific T cells in which the antigen was present in the corresponding reaction mixture.

In some embodiments, the present disclosure provides a T cell receptor (TCR) that binds a KRAS G12D neoantigen comprising a TCR-alpha chain and a TCR-beta chain, the TCR-alpha variable region comprises a CDR1 comprising the amino acid sequence SEQ ID NO: 7, a CDR2 comprising the amino acid sequence SEQ ID NO: 8, and a CDR3 comprising amino the acid sequence SEQ ID NO: 9; and wherein the TCR-beta variable region comprises the following: a CDR1 comprising the amino acid sequence SEQ ID NO: 3, a CDR2 comprising the amino acid sequence SEQ ID NO: 4, and a CDR3 comprising the amino acid sequence SEQ ID NO: 6, and, wherein the antigen specificity of the TCR or portion thereof is determined by a method comprising the steps of: dividing a tissue sample into a first subset and a second subset; sequencing recombined nucleic acids encoding a TCR or a portion thereof from the first subset to provide sequence reads from which clonotypes are determined; exposing the second subset to KRAS G12D neoantigen; separating T cells from the second subset that interact with the antigen to obtain an enriched T cell sample; sequencing recombined nucleic acids encoding a TCR or a portion thereof from the enriched T cell sample to provide sequence reads from which clonotypes are determined; and identifying KRAS G12D neoantigen-specific T cells in the tissue sample as T cells whose clonotype frequencies increase in the enriched T cell sample relative to the frequencies of the same clonotype in the first subset.

In some embodiments, the present disclosure provides a T cell receptor (TCR) that binds a KRAS G12D neoantigen comprising a TCR-alpha chain and a TCR-beta chain, the TCR-alpha variable region comprises a TCR-alpha chain variable region and a TCR-beta chain variable region wherein the TCR comprises (a) a TCR-alpha chain variable region comprising a CDR1, a CDR2, and a CDR3 comprising amino acid sequences SEQ ID NOs: 19, 20, and 21, respectively, and a TCR-beta chain variable region comprising a CDR1, a CDR2, and a CDR3 comprising amino acid sequences SEQ ID NOs: 16, 17, and 18, respectively; or (b) a TCR-alpha chain variable region comprising a CDR1, a CDR2, and a CDR3 comprising amino acid sequences SEQ ID NOs: 29, 30, and 31, respectively, and a TCR-beta chain variable region comprising a CDR1, a CDR2, and a CDR3 comprising amino acid sequences SEQ ID NOs: 26, 27, and 28, respectively; or (c) a TCR-alpha chain variable region comprising a CDR1, a CDR2, and a CDR3 comprising amino acid sequences SEQ ID NOs: 39, 40, and 41, respectively, and a TCR-beta chain variable region comprising a CDR1, a CDR2, and a CDR3 comprising amino acid sequences SEQ ID NOs: 36, 37, and 38, respectively; or (d) a TCR-alpha chain variable region comprising a CDR1, a CDR2, and a CDR3 comprising amino acid sequences SEQ ID NOs: 49, 50, and 51, respectively, and a TCR-beta chain variable region comprising a CDR1, a CDR2, and a CDR3 comprising amino acid sequences SEQ ID NOs: 46, 47, and 48, respectively; or (e) a TCR-alpha chain variable region comprising a CDR1, a CDR2, and a CDR3 comprising amino acid sequences SEQ ID NOs: 59, 60, and 61, respectively, and a TCR-beta chain variable region comprising a CDR1, a CDR2, and a CDR3 comprising amino acid sequences SEQ ID NOs: 56, 57, and 58, respectively; or (f) a TCR-alpha chain variable region comprising a CDR1, a CDR2, and a CDR3 comprising amino acid sequences SEQ ID NOs: 69, 70, and 71, respectively, and a TCR-beta chain variable region comprising a CDR1, a CDR2, and a CDR3 comprising amino acid sequences SEQ ID NOs: 66, 67, and 68, respectively; or (g) a TCR-alpha chain variable region comprising a CDR1, a CDR2, and a CDR3 comprising amino acid sequences SEQ ID NOs: 79, 80, and 81, respectively, and a TCR-beta chain variable region comprising a CDR1, a CDR2, and a CDR3 comprising amino acid sequences SEQ ID NOs: 76, 77, and 78, respectively; or (h) a TCR-alpha chain variable region comprising a CDR1, a CDR2, and a CDR3 comprising amino acid sequences SEQ ID NOs: 89, 90, and 91, respectively, and a TCR-beta chain variable region comprising a CDR1, a CDR2, and a CDR3 comprising amino acid sequences SEQ ID NOs: 86, 87, and 88, respectively, wherein the antigen specificity of the TCR or portion thereof is determined by a method comprising the steps of: dividing a tissue sample into a first subset and a second subset; sequencing recombined nucleic acids encoding a TCR or a portion thereof from the first subset to provide sequence reads from which clonotypes are determined; exposing the second subset to KRAS G12D neoantigen; separating T cells from the second subset that interact with the antigen to obtain an enriched T cell sample; sequencing recombined nucleic acids encoding a TCR or a portion thereof from the enriched T cell sample to provide sequence reads from which clonotypes are determined; and identifying KRAS G12D neoantigen-specific T cells in the tissue sample as T cells whose clonotype frequencies increase in the enriched T cell sample relative to the frequencies of the same clonotype in the first subset.

In some embodiments, viable antigen-specific T cells can be obtained based on acquisition of the cell surface markers CD137/107 (for CD8 antigen-specific T cells) following brief in vitro incubation with peptides as taught by, e.g. Chattopadhyay et al, Nature Medicine, 11: 1113-1117 (2005); Meier et al, Cytometry A, 73: 1035-1042 (2008); Wolfl et al, Blood, 110: 201-210 (2007); Wolfl et al, Cytometry A, 73: 1043-1049 (2008); and the like.

### VII. Identifying Paired T Cell Receptor Chains without Antigen-Specific Selection

In one aspect, the invention provides methods for matching pairs of immune receptor chains from populations of their encoding nucleic acids that have been sequenced. In some embodiments, the invention provides for methods for determining functional KRAS G12D-specific TCRs from subunits selected from separate libraries. In some embodiments, the methods used for determining nucleic acids that encode KRAS G12D-specific TCR chains originating from the same cell is as taught by Faham, et al, U.S. Pat. No. 10,077,478. In some embodiments, the method used is pairSeq as taught by Howie, et al, Sci Transl Med. 2015 Aug. 19; 7(301):301ra131, Erratum in: Sci Transl Med. 2015 Oct. 14; 7(309):309er7.

In one approach, which is applicable to matching all three types of immune receptor pairs, a lymphocyte population is repeatedly divided into a plurality of subsets. Such subsets may be obtained by aliquoting a tissue sample into separate reaction vessels or chambers. Separately from each of a portion, or subpopulation, of the subsets, nucleic acids encoding the two different immune receptor chains are extracted and sequenced, so that two separate lists of sequences are formed without any correspondence between members of each list. As described above, this may be achieved by carrying out separate sequencing runs for each chain, or it may be accomplished by carrying out a single sequence run with the nucleic acids tagged according to the identity of the type of chain it encodes. To illustrate by an example, if a sample containing T cells is aliquoted into 100 sub-samples, so that on average each aliquot contains a subset consisting of about 1/100 of the total number of T cells in the original sample, then 20 such subsets may be randomly selected as a portion of the total number of subsets. (Such portion could be any number greater than one and less than 100, although as described more fully below, a number in the range of from 10 to 20 is a good trade-off between amount of sequencing required and likelihood of identifying receptor pairs present at a frequency of interest). In one embodiment, a plurality of subsets is in the range of from 20 to 2000 and a portion of subsets thereof is in the range of from 10 to 50. In another embodiment, a portion of subsets is in the range of from 10 to 20.

In one aspect, the above embodiment may be carried out by the following steps: (a) obtaining a sample containing T cells; (b) determining nucleotide sequences of TCR alpha chains of T cells from the sample, each TCR alpha chain having a frequency of occurrence in the sample; (c) determining nucleotide sequences of TCR beta chains of T cells from the sample, each TCR beta chain having a frequency of occurrence in the sample; and (d) identifying paired TCR alpha chains and TCR beta chains as those having the same frequency within the sample. Frequencies of the respective TCR alpha chains and TCR beta chains may be determined from the tabulations of encoding nucleic acids, or clonotypes. Alternatively, frequencies of the respective TCR alpha chains and TCR beta chains may be determined from the tabulations of polypeptides encoded by the clonotypes. As mentioned above, clonotype frequencies may be determined by counting clonotypes directly or indirectly by using a tagging scheme as described above.

Another embodiment for identifying matching receptor subunits which may be applied to TCRs and may be used even when receptor frequencies among subunit chains are close or indistinguishable, whether because of experimental error or otherwise. Starting with a sample containing lymphocytes, which may be T cells, subsets are formed by separating or partitioning the sample into a plurality of subsets, 1 through K. In some embodiments, only a portion of the K subset are analyzed; thus, it is not necessary to actually form all K subsets. One may form subsets of only the portion that are actually analyzed. For example, if the sample has a volume of 100 μL and K=100, but only a portion consisting of 20 subset is to be analyzed, then only twenty 1 μL subsets need be formed. From each subset nucleic acids encoding each different immune receptor chain (TCR alpha and TCR beta being under subset 1) are sequenced, thereby forming pairs of lists. Each pair of such lists contains a first list of nucleotide sequences of a first immune receptor chain, e.g. list for TCR alpha of subset 1, and a second list of nucleotide sequences of a second immune receptor chain, e.g. list for TCR beta of subset 1. In one embodiment, the number of subsets, K, is a number in the range of from 5 to 500; in another embodiment, K is a number in the range of from 10 to 100; in another embodiment, K is a number in the range of from 20 to 50. In some embodiments, a portion of subsets analyzed is 10 or fewer subsets; in other embodiments, a portion of subsets analyzed is 20 or fewer subsets; in other embodiments, a portion of subsets analyzed is at least five percent of the subsets; in other embodiments, a portion of subsets analyzed is at least ten percent of the subsets; in other embodiments, a portion of subsets analyzed is at least twenty percent of the subsets.

Each kind of lymphocyte in sample, e.g. lymphocyte, is present in the sample at a particular frequency. The distribution of lymphocytes into the subsets is readily approximated by a binomial model; thus, for an arbitrary lymphocyte having a particular clonotype, (a) its frequency in the sample, (b) the total number of lymphocytes in the sample, and (c) the number of subsets may be related to the expectation of finding at least one of the particular lymphocyte in a predetermined fraction of subsets. This relationship may be expressed as follows: r=(1−f)^{(N/K)}, where r is the fraction of subsets containing at least one of the particular lymphocyte, f is the frequency of the particular lymphocyte in the sample, Nis the total number of lymphocytes in the sample, and K is the number of subsets. Thus, if one sets r=½ and takes N as a constant, then one may select successive values of K so that lymphocytes of different frequencies are present in about half of the subsets. Other values of r could be selected, but r=½ provides results with the highest statistical power, thus the value r≈½ is preferred. Once such lists are obtained they are examined to identify pairs of first and second nucleotide sequences that either occur in a subset together or are both absent from a subset.

In one aspect of the invention, matched first and second chains of lymphocytes from a succession of frequency classes may be determined by carrying out the above process repeatedly for different values of K. For example, a 1 mL sample of peripheral blood of a normal individual contains about 1-4.8×10⁶ lymphocytes of which about 10-15 percent are B cells, about 70-85 percent are T cells and about 10 percent are NK cells; thus, the 1 mL sample may contain from about 7×10⁵ to about 4×10⁶ T cells. If the number of T lymphocytes in a 1 mL sample is N=10⁶, then matching TCR chains of T cells of the following frequencies are matched by identifying those that appear together in fifty percent of the subsets and not at all in the other fifty percent of subsets:

TABLE 1

| Frequency | Number of Subsets | Volume (μL) |
|---|---|---|
| .001 | 1443 | 0.7 |
| .0005 | 722 | 1.4 |
| .0001 | 144 | 6.9 |
| .00005 | 72 | 13.9 |

As mentioned above, not all the subsets at a particular frequency need be analyzed. If there are a large number of lymphocytes that have frequencies at or close to a selected frequency, e.g. f=0.001, they may all be resolved by taking a larger and larger portion of the total number of subsets until every pair that appears together in fifty percent of the subsets can be distinguished from every other pair at the same frequency. This is because the probability of two different lymphocytes occurring in exactly the same subsets of the fifty percent becomes infinitesimal as the portion of subsets is increased.

### VIII. Identifying Paired and Unpaired T Cell Receptor Chains with Antigen-Specific Selection

In some embodiments, the invention is directed to identifying antigen-specific T cells by one or a pair of immune receptor chains, such as TCR alpha, or TCR beta, or TCR alpha and TCR beta together; or TCR delta, or TCR gamma, or TCR delta and TCR gamma together. In some embodiments, the nucleotide sequence encoding a single immune receptor chain, such as TCR beta, is used to identify antigen-specific T cells. Sometimes such nucleotide sequences are referred to herein as a “clonotype,” although clonotypes also may be ordered pairs of nucleotide sequences specific to a particular T cell, such as the nucleotide sequences encoding the T cell's TCR alpha and TCR beta chains, which may be represented (for example) as (Sa, SB), or like notation, where Sa is a sequence of a segment of TCR alpha and Sp is a sequence of a segment of TCR beta, and as a pair they are a clonotype of the cell they originate from.

In some embodiments, to a tissue sample comprising T cells is added antigen under interaction conditions so that T cells specific for antigen may interact with antigen. Such interaction may be direct or indirect. Direct interactions include binding of antigen to antigen-specific T cells, binding of antigen peptide-multimer conjugates to antigen-specific T cells, and the like. Peptide-multimer conjugates, such as tetramers, are well-known reagents to those of ordinary skill, e.g. Bousso, Microbes Infect. 2(4): 425-429 (2000); Klenerman et al, Nature Reviews Immunol., 2(4): 263-272 (2002); and the like. Indirect interactions include presentation of antigen or antigen peptides to antigen-specific T cells by antigen presenting cells, such as, dendritic cells, artificial APCs, and the like. In some interactions, antigen-specific T cells may become activated T cells that may proliferate and/or develop or express activation markers both of which provide means for selecting and/or enriching antigen-specific T cells using conventional techniques. Antigen may comprise a wide variety of compounds or compositions as discussed more fully below. Proteins and peptides derived from one or more proteins are of special interest, particularly when the proteins are associated with cancers or infectious diseases, such as bacterial or virus infections. Antigen may be combined with, exposed to, or added to, tissue sample in a variety of ways known in the art, e.g. Berzofsky et al, J. Clin. Investigation, 113: 1515-1525 (2004). After combining antigen with tissue sample in a reaction mixture, antigen-specific T cells and non-antigen-specific T cells alike are exposed to antigen with which they interact either directly or indirectly.

In some embodiments, antigen-specific T cells are activated, possibly after a period of incubation with antigen. A period of incubation may vary widely. In some embodiments, incubation may be for an interval of from a few minutes (for example, 10 minutes) to an hour or more; in other embodiments, incubation may be for an interval of a few hours (for example, 2 hours) to 8 or more hours. In other embodiments, antigen-specific T cells interact with antigen by binding to or forming complexes with antigen or antigen reagents, such as antigen peptide-multimer conjugates, such that activation may not take place. A step of exposing may include the step of incubating a tissue sample with an antigen. For example, in the case of a protein antigen and a tissue sample that comprises PBMCs, a step of exposing may include combining the tissue sample with peptides derived from the protein antigen such that dendritic cells in the tissue sample present the peptides to antigen-specific T cells in the tissue sample which, in turn, interact with the antigen-presenting dendritic cells and are activated. After exposing T cells to antigen so that antigen-specific T cells interact with antigen, antigen-specific T cells may be selected and/or enriched based on some feature resulting from the interaction, such as antigen peptide-multimer binding, activation markers induced, proliferation of the T cells, or the like. As mentioned above, the step of selecting antigen-specific T cells may be alternatively a step of enriching antigen-specific T cells from the reaction mixture, and/or a step of separating antigen-specific T cells from the reaction mixture, and/or a step of isolating antigen-specific T cells from the reaction mixture. After antigen-specific T cells are enriched, separated, and/or isolated their clonotypes are determined by sequencing a predetermined segment of a recombined nucleic acid that encodes a portion of an immune receptor, such as TCR beta and/or TCR alpha.

A predetermined segment chosen may vary widely; in some embodiments, it encompasses all or a portion of a V (D) J region, so that clonotypes based thereon have maximal diversity for unique identification of cell clones. Determination of clonotypes is described more fully below, but briefly, recombined nucleic acids encoding one or more selected immune receptors (such as TCR beta) are sequenced (for example, by spatially isolating molecules thereof, amplifying such molecules, and carrying out sequencing steps by a high-throughput sequencing chemistry, such as available with commercial next-generation DNA sequencers). As a result of these sequencing steps, sequence reads are produced which are used to determine clonotypes and clonotype frequencies of antigen-specific T cells. Clonotypes and clonotype frequencies are also determined either for T cells of the tissue sample from sequence reads or for non-antigen-specific T cells from sequence reads. Non-antigen-specific T cells may be obtained from a two-way sorting procedure (for example, using FACS or MACS) based on T cells labeled according to an interaction, such as, an interaction of antigen-specific T cells with fluorescently labeled antigen peptide multimers. These data may then be analyzed to identify clonotypes associated with antigen-specific T cells, for example. Briefly, in some embodiments, antigen-specific T cells may be associated with clonotype frequencies that increase in the selected population of T cells relative to frequencies of the same clonotype in populations of non-antigen specific T cells or in the population of T cells in tissue sample.

Exemplary steps for implementing this embodiment of the invention (i.e., for determining clonotypes associated with antigen-specific T cells in a tissue sample) may include the following: (a) exposing the T cells of the sample to an antigen so that T cells specific for the antigen interact with the antigen; (b) sequencing recombined nucleic acids encoding a T cell receptor chain or a portion thereof from a sample of T cells from the tissue sample to provide sequence reads from which clonotypes are determined; (c) isolating antigen-specific T cells from the tissue sample based on their interaction with the antigen; (d) sequencing recombined nucleic acids encoding a T cell receptor chain or a portion thereof from a sample of the isolated antigen-specific T cells to provide sequence reads from which clonotypes are determined; and (e) determining antigen-specific T cells in the tissue sample as T cells whose clonotype frequencies increase in the sample of isolated T cells relative to the frequencies of the same clonotypes in a sample of T cells in the tissue sample.

In some embodiments, a step of exposing may be carried out by reacting under interaction conditions an antigen with a tissue sample; in still other embodiments, a step of exposing may be carried out by reaction under activation conditions an antigen with a tissue sample. As mentioned above the step of exposing for this and other embodiments may vary widely, and its implementation may depend on the nature of the tissue sample and the nature of the antigen, as well as other factors. For example, if a tissue sample includes antigen-presenting cells, such as dendritic cells, then exposing may include either addition of an antigen, such as a protein, directly to the tissue sample, or it may include producing antigenic material from an antigen of interest followed by addition of the antigenic material. More efficient T cell activation to a protein antigen, for example, may be accomplished by exposing a tissue sample to a set of overlapping peptides derived from the protein antigen of interest, using conventional techniques. Alternatively, artificial antigen-presenting compositions may be used in the exposing step or its equivalent, e.g. Oelke et al, Nature Medicine, 9(5): 619-624 (2003).

The step of exposing T cells in a tissue sample may include exposing such T cells to whole cells containing antigen, to gene-modified cells expressing antigen, to whole protein, to peptides derived from a protein antigen, to viral vectors expressing an antigen, to antigen-modified, or loaded, dendritic cells. In some embodiments, a tissue sample is a blood sample; in other embodiments, a tissue sample is a sample of peripheral blood mononuclear cells (PBMCs) derived from peripheral blood using conventional techniques. In some embodiments the step of exposing may be carried out by reacting under activation conditions a tissue sample comprising T cells with an antigen, where various activation conditions are described above. In view of the wide variety of tissue samples and antigens, the step of exposing may be alternatively carried out by a step of reacting under activation conditions a tissue sample comprising T cells with an antigen.

Further exemplary steps for implementing the above method may comprise: (a) reacting under activation conditions a tissue sample comprising T cells to an antigen; (b) sorting from the tissue sample activated T cells and un-activated T cells; (b) sequencing recombined nucleic acids encoding a T cell receptor chain or a portion thereof from a sample of T cells from the activated T cells to provide sequence reads from which clonotypes are determined; (c) sequencing recombined nucleic acids encoding a T cell receptor chain or a portion thereof from a sample of T cells from the un-activated T cells to provide sequence reads from which clonotypes are determined; and (d) determining antigen-specific T cells in the tissue sample as T cells whose clonotype frequencies increase in the sample of activated T cells relative to the frequencies of the same clonotypes in the tissue sample or in a sample of un-activated T cells. Likewise, exemplary steps for implementing the above method may comprise: (a) reacting under interaction conditions a tissue sample comprising T cells with an antigen; (b) sorting T cells of the tissue sample into a first subset of T cells that form complexes with the antigen or antigen reagents thereof and into a second subset of T cells that do not form complexes with the antigen or antigen reagents thereof; (b) sequencing recombined nucleic acids encoding a T cell receptor chain or a portion thereof from a sample of the first subset to provide sequence reads from which clonotypes are determined; (c) sequencing recombined nucleic acids encoding a T cell receptor chain or a portion thereof from a sample of T cells from the tissue sample or the second subset to provide sequence reads from which clonotypes are determined; and (d) determining antigen-specific T cells in the tissue sample as T cells whose clonotype frequencies increase in the sample of T cells of the first subset relative to the frequencies of the same clonotypes in the tissue sample or in a sample of T cells from the second subset. As used herein, the term “antigen reagents” means reagents derived from an antigen designed to bind to, or form complexes with, T cells whose TCRs are specific for the antigen. Exemplary antigen reagents include, but are not limited to, multimers conjugated with peptides derived from an antigen.

In some embodiments, the above method of determining antigen-specific T cells in a tissue sample may be carried out by steps comprising: (a) reacting under activation conditions in a reaction mixture a tissue sample comprising T cells to an antigen or antigen reagents thereof; (b) sequencing recombined nucleic acids encoding a T cell receptor chain or a portion thereof from a sample of T cells from the reaction mixture prior to addition of the antigen to the reaction mixture to provide sequence reads from which clonotypes are determined; (c) incubating the reaction mixture after addition of the antigen or antigen reagent thereof for a predetermined interval; (d) sequencing recombined nucleic acids encoding a T cell receptor chain or a portion thereof from a sample of T cells from the incubated reaction mixture to provide sequence reads from which clonotypes are determined; (d) determining antigen-specific T cells in the tissue sample as T cells whose clonotype frequencies increase in the incubated reaction mixture relative to the frequencies of the same clonotypes in the reaction mixture prior to the addition of antigen. In some embodiments, a predetermined interval for incubation is usually greater than eight hours; in other embodiments, a predetermined interval may be greater than 24 hours; in further embodiments, a predetermined interval may be within a range of from 8 hours to 72 hours.

In some embodiments, step of isolating antigen-specific T cells may be substituted with either a step of separating a sample of antigen-specific T cells from the tissue sample after exposure to an antigen of interest or a step of recovering antigen-specific T cells from the tissue sample after exposure to an antigen of interest. In some embodiments, such step of isolating may be carried out by sorting antigen-interacting and/or activated T cells from a tissue sample; likewise, in some embodiments, non-antigen-specific T cells and/or unactivated T cells may be sorted from a tissue sample. Such steps of the various embodiments may be carried out by a variety of methods including, but not limited to, (i) peptide-MHC multimer staining reagents (such as, tetramers, pentamers, or the like), followed by sorting, panning, or otherwise capturing complexes between such reagents and antigen-specific T cells, (ii) sorting or panning or capturing based on activation markers, such as CD137, CD154, or others (described more fully below), or (iii) proliferation (and therefore, for example, an increase in frequency) of antigen-specific T cells over antigen-non-specific T cells. Thus, in some embodiments, said step of isolating may comprise a step isolating activated T cells; or a step of separating activated T cells from the tissue sample. In some of such embodiments, T cell activation markers, as noted above, may be used to sort, pan or otherwise capture activated T cells, using conventional techniques. Generally, a step is taken for obtaining a sample of T cells from a pool of T cells derived from the tissue sample, which pool is enriched in antigen-specific T cells and/or activated T cells. In some embodiments, T cells with an activation marker may be sorted or isolated using a binding compound, such as an antibody, which specifically binds to the activation marker and which can be directly or indirectly labeled in accordance with conventional methods, e.g. FACS, magnetic bead-based separation, or like techniques.

In one aspect, a similarity measure for use with these embodiments of the invention is a monotonically varying function that maps (or is capable of mapping by a simple transformation) at least two sets of clonotype frequency measurements (e.g. two sequence-based clonotype profiles) to the unit interval [0, 1]. Simple transformations include, but are not limited to, any linear transformation of dependent variables, logarithmic transformations, such as yᵢⱼ=ln(nᵢⱼ+1) (where nᵢⱼ is the number of clonotype i in sample j), or the like. A value of zero means no similarity between clonotype profiles and a value of one means two clonotype profiles are statistically identical.

Exemplary similarity measures that may be implemented in these embodiments are described in Legendre and Legendre, Numerical Ecology (Elsevier, 1998); Magurran, Measurement of Biological Diversity (Wiley-Blackwell, 2003); Wolda, Occologia (Berl), 50: 296-302 (1981). Such similarity measures include, but are not limited to, Czekanowski's index, Dice's coefficient, Horn's information theory index, Canberra metric, Morisita's index, Kaczynski's similarity index, Sorensen's index, Jacquard's index, Bray-Curtis index, and the like. In one aspect, similarity measures are similarity metrics; or in other words, the similarity measures employed have properties of a distance measure, such as, (i) the value of the measure is always non-negative, (ii) the measure is zero if and only if the clonotype profile measurements are identical, (iii) the value of the measure is invariant with respect to the ordering of the clonotype profile measurements (sometimes expressed as d(x,y)=d(y,x)), (iv) the triangle inequality holds with respect to three different clonotype profile measurements. In another aspect, similarity measures may be correlation coefficients (subject to a simple transformation, e.g. taking its absolute value, squaring its value, or the like, so that its value is restricted to the unit interval). Exemplary correlation coefficients include, but are not limited to, Pearson product-moment correlation coefficient and rank correlations, such as Spearman's rank correlation coefficient, Kendall's tau rank correlation coefficient, and the like. In one embodiment a Morisita-Horn index (C₁₂) (including Morisita-Horn index with a logarithmic transformation), as disclosed in Wolda (cited above), is employed with the embodiments.

In another embodiment for identifying pairs of immune receptor chains of antigen-specific T cells, T cell containing reaction mixtures are exposed to a single antigen. A tissue sample is partitioned into subsets from 1 to K and a portion of the subsets may be selected for analysis. In one embodiment, as above, the partitions may be aliquots of the tissue sample, in which approximately equal amounts of tissue sample are provided to each subset, for example, by distributing equal amounts of tissue sample to each of K reaction mixtures, which may be contained by vessels or reactors, such as wells in a multi-well plate. Tissue samples may also be distributed to a plurality of K separate chambers of a microfluidics device in connection with this and/or the embodiments described above. T cells of each subset are exposed to antigen after which reaction mixtures in the K vessels are incubated for a time (for example, a predetermined interval) sufficient for T cells to respond to, or interact with, the antigen, either directly or in a processed form (for example, as an antigen reagent). Such response may include forming a stable complex with antigen or a processed form thereof, or may include the development and/or expression of activation markers by T cells, or may include proliferation by T cells specific for the antigen. Antigen-interacting or antigen-responding T cells are then selected and isolated (for example, sorted) from each of the K chambers, after which recombined nucleic acids encoding predetermined portions of one or both TCR chains are sequenced to provide sequence reads from which clonotypes and clonotype profiles are formed. As above with the embodiment of FIG. 12B, once such profiles are obtained they are examined to identify pairs of first and second nucleotide sequences that either occur in a subset together or are both absent from a subset. By way of example, the members of pair appear in lists of subset 2 and in lists of subset K−1, but neither member of the pair appears in lists or of subsets 1 and K, either alone or together. As above, this reflects the presence or absence of a particular lymphocyte, which in this illustration is in subsets 2 and K−1, but is absent from subsets 1 and K. Such a pattern confirms that the members of pair go together and correspond to the chains of a functional immune receptor that is specific for antigen, Ag₁.

In some embodiments, the above method of determining receptors of antigen-specific T cells in a tissue sample may comprise the following steps: (a) partitioning a tissue sample containing T cells into a plurality of subsets; (b) exposing the T cells of each of a portion of subsets to an antigen so that T cells specific for the antigen are activated; (c) isolating the activated T cells of each subset of the portion; (d) sequencing recombined nucleic acids encoding T cell receptor a chains in each subset of the portion to provide sequence reads from which a chain clonotypes are determined; (e) sequencing recombined nucleic acids encoding T cell receptor beta chains in each subset of the portion to provide sequence reads from which beta chain clonotypes are determined; and (f) identifying as antigen-specific T cell receptors with those pairs of alpha chain clonotypes and beta chain clonotypes that for every subset of the portion (i) either both the a chain clonotype and beta chain clonotype are present in a subset or neither are present in a subset, and (ii) both the a chain clonotype and beta chain clonotype are present in at least one subset of the portion and the a chain clonotype and beta chain clonotype are not present in at least one subset of the portion.

Alternatively, in some embodiments, the above method of determining receptors of antigen-specific T cells in a tissue sample may comprise the following steps: (a) forming a plurality of subsets from a tissue sample containing T cells; (b) reacting under activation conditions the T cells of each subset to an antigen; (c) isolating the antigen-specific T cells of each subset; (d) sequencing recombined nucleic acids encoding T cell receptor a chains in each subset to provide sequence reads from which a chain clonotypes are determined; (e) sequencing recombined nucleic acids encoding T cell receptor beta chains in each subset to provide sequence reads from which beta chain clonotypes are determined; (d) identifying as antigen-specific T cell receptors with those pairs of a chain clonotypes and beta chain clonotypes that for every subset (i) either both the alpha chain clonotype and beta chain clonotype are present in a subset or neither are present in a subset, and (ii) both the a chain clonotype and beta chain clonotype are present in at least one subset and the a chain clonotype and beta chain clonotype are not present in at least one subset. In some of these latter embodiments, the plurality of subsets formed may correspond to a portion of the plurality into which a tissue sample is partitioned in the former embodiments. In some embodiments, the step of forming a plurality of subsets may comprise aliquoting portions of a tissue sample into separate reaction vessels. In some embodiments, such portions are equal portions.

### IX. Methods of Treatment

The present disclosure provides methods for treating KRAS G12D-expressing cancer, wherein the methods comprise administering to a subject a pharmaceutical composition of recombinant T cells expressing one or more of the TCRs provided herein. In some embodiments, the cancer is a solid tumor, such as pancreatic ductal adenocarcinoma, colorectal cancer, or non-small cell lung cancer. In some embodiments, the method comprises obtaining cells (e.g., immune cells as provided herein) from the subject to be treated or from a cell donor source; introducing one or more nucleic acid encoding one or more of the TCRs provided herein into the cells such that the cells exogenously express the TCRs provided herein; optionally further expanding the cells; and transferring the cells to the subject. Thus, in some embodiments, the methods provided herein are autologous cell transfer methods, and in other embodiments the methods provided herein are allogenic cell transfer methods. In some embodiments, the cells transferred to the subject comprise more than one TCR provided herein. For example, in some embodiments, the cells transferred to the subject are present in a population of cells wherein each cell comprises 1, 2, 3, 4, 5, or 6 different exogenous TCRs. In other embodiments, the cells transferred to the subject are present in a population of cells made up of 1, 2, 3, 4, 5, or 6 subpopulations of cells, wherein each subpopulation of cells comprises 1, 2, 3, 4, 5, or 6 different exogenous TCRs.

Cells comprising TCRs reactive against the KRAS G12D neoantigen epitope are suitable for use in adoptive transfer methods to provide treatment to a subject in need of treatment for KRAS G12D-expressing cancer. The approach to such cell therapy generally comprises adoptively transferring to a subject in need thereof isolated cells expressing one or more of the TCRs provided herein under conditions permissive for expression of the TCR in the subject, as will be known to those of skill in the art. Thus, the present disclosure provides methods for treating KRAS G12D-expressing cancer, comprising adoptively transferring to a subject in need thereof isolated cells recombinantly expressing one or more of the TCRs provided herein.

In that respect, the present disclosure provides methods for adoptive transfer of cells comprising the TCRs provided herein. Cells may be isolated from a subject using any method known in the art. For example, cells may be isolated using an isolation kit, Ficoll-Paque density gradient centrifugation, flow cytometer cell sorting, and the like. In some embodiments, isolated cells may be autologous (i.e., derived from the subject that will receive the resultant transduced or transformed cells). For example, the isolated cells may be obtained from PBMCs and/or hematopoietic stem cells of the subject. In other embodiments, isolated cells may be allogenic. In some embodiments, the isolated cell may be an immune cell. The immune cell may be a T cell, which may be a naïve T cell, an effector T cell, a central memory T cell, an effector memory T cell, a CD4+ T cell, a CD8+ T cell, an alpha/beta T cell, a gamma/delta T cell, a regulatory T cell, or any combination thereof. In some embodiments, the isolated cell may be a T cell, such as a CD4+ T cell or a CD8+ T cell. In particular embodiments, the isolated T cell is a CD8+ T cell. CD8+ T cells are also known as cytolytic T cells (CTLs). In some embodiments, the isolated T cells are expanded in vitro after separation from the subject. During expansion, the isolated T cells may be incubated with accessory cells (e.g., PBMC, dendritic cells, B cells, or monocytes) to support expansion of the T cells in vitro prior to transfer to a subject. In some embodiments, unexpanded isolated T cells are transferred, and such T cells may expand and become activated in vivo.

### A. Cancers

In some embodiments, the recombinant TCRs, engineered T cells, or pharmaceutical compositions of the present invention are used in methods of treating cancer. In some embodiments, the subject (also referred to as “the patient”) receiving treatment with the engineered cells or pharmaceutical composition of the invention is HLA-A*11:01-positive. In some embodiments, the patient is KRAS G12D mutation-positive.

In some embodiments, the cancer to be treated is pancreatic ductal adenocarcinoma. In some embodiments, the cancer to be treated is colorectal cancer. In some embodiments, the cancer to be treated is non-small cell lung cancer. These cancers are associated with poor survival rates and limited treatment options. In addition, these cancers are associated with KRAS mutations, including the KRAS G12D mutation.

In some embodiments, the cancer manifests as a solid tumor. In some embodiments the cancer may be a carcinoma, a blastoma, or a sarcoma. In some alternate embodiments, the cancer may be a liquid tumor, such as a leukemia or a lymphoma. In some embodiments, the leukemia or lymphoma is acute T cell lymphoblastic leukemia/lymphoma (T-ALL), juvenile myelomonocytic leukemia, or myelodysplastic/myeloproliferative neoplasm.

In some embodiments, the solid tumor cancer is squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, or various types of head and neck cancer (including squamous cell carcinoma of the head and neck). In some embodiments, the cancer is pancreatic cancer. In some embodiments, the solid tumor cancer is gastric cancer. In some embodiments, the solid tumor cancer is prostate cancer. In some embodiments, the solid tumor cancer is endometrial cancer. In some embodiments, the solid tumor cancer is non-small cell lung cancer. In some embodiments, the solid tumor cancer is colorectal cancer. In some embodiments, the solid tumor cancer is ovarian cancer.

In some embodiments, the cancer is advanced. In some embodiments, the cancer is relapsed. In some embodiments, the cancer is refractory. In some embodiments, the cancer is metastatic. In some embodiments, the cancer is a solid tumor, which may be an advanced solid tumor. In some embodiments, the cancer is a relapsed solid tumor. In some embodiments the cancer is a refractory solid tumor. In some embodiments, the cancer is a metastatic solid tumor. In some embodiments, the cancer is an advanced, and/or relapsed, and/or refractory, and/or metastatic solid tumor.

In some embodiments, the subject having cancer has experienced disease progression during or after standard therapy. In some embodiments, the subject having cancer was intolerant of standard therapy. In some embodiments, the subject having cancer does not have appropriate therapies available to them based on the judgment of a treating physician.

### B. Patient Selection Criteria

In some embodiments, the subject (also referred to as “the patient”) receiving treatment with the engineered cells or pharmaceutical composition of the invention is HLA-A*11:01-positive. In some embodiments, the patient is KRAS G12D mutation-positive. In some embodiments, the patient has cancer, such as PDAC, CRC, or NSCLC. In some embodiments, the patient has histologic or cytologic documentation of PDAC, CRC, or NSCLC.

In some embodiments, for patients with PDAC treated with the recombinant TCR, engineered cells, or pharmaceutical composition of the invention, one, some, or all of the following criteria are met: histologically or cytologically confirmed metastatic PDAC, disease progression with or intolerance to either 5FU- or gemcitabine-based first-line chemotherapy, and patients with endocrine or acinar pancreatic carcinoma are not eligible for the study.

In some embodiments, for patients with CRC treated with the recombinant TCR, engineered cells, or pharmaceutical composition of the invention, one, some, or all of the following criteria are met: histologically confirmed metastatic adenocarcinoma of the colon or rectum, mismatch repair (MMR)/microsatellite instability (MSI) status must be known, patients with dMMR/MSI-H should have received prior treatment with an approved immunotherapy (e.g., anti-PD-1/anti-PD-L1 with or without anti-CTLA4 therapy), and disease progression with or intolerance to a fluoropyrimidine-based regimen that includes either oxaliplatin (e.g., FOLFOX, CAPEOX) or irinotecan (e.g., FOLFIRI).

In some embodiments, for patients with NSCLC treated with the recombinant TCR, engineered cells, or pharmaceutical composition of the invention, one, some, or all of the following criteria are met: histologically confirmed unresectable, locally advanced or metastatic adenocarcinoma of the lung, disease progression with or intolerance to single-agent or combination therapy with an investigational or approved PD-L1/PD-1 inhibitor, and patients whose tumors have a targetable somatic alteration, including those involving EGFR, ALK, ROS1, BRAFV600E, NTRK, MET, RET and KRAS G12C must have experienced disease progression with or intolerance to treatment with a targeted agent.

### C. Atezolizumab

In some embodiments, the methods described herein further comprise treatment of cancer in a subject in need thereof, comprising administering a combination of a recombinant TCR that binds to a KRAS G12D neoantigen, an engineered T cell comprising said recombinant TCR, or a pharmaceutical composition comprising said engineered T cells, and an anti-PD-L1 antibody. In some embodiments, the anti-PD-L1 antibody is atezolizumab.

Atezolizumab is a humanized IgG1 monoclonal antibody that targets PD-L1 and inhibits the interaction between PD-L1 and its receptors, PD-1 and B7-1 (also known as CD80), both of which function as inhibitory receptors expressed on T cells. Therapeutic blockade of PD-L1 binding by atezolizumab has been shown to enhance the magnitude and quality of tumor-specific T cell responses, resulting in improved anti-tumor activity (Fehrenbacher et al, “Atezolizumab versus docetaxel for patients with previously treated non-small-cell lung cancer (POPLAR): a multicentre, open-label, phase 2 randomised controlled trial,” Lancet 2016; 387:1837046; Rosenberg et al, “Atezolizumab in patients with locally advanced and metastatic urothelial carcinoma who have progressed following treatment with platinum-based chemotherapy: a single-arm, multicentre, phase 2 trial,” Lancet 2016; 387:1909□20). Atezolizumab has minimal binding to Fc receptors, thus eliminating detectable Fc effector function and associated antibody-mediated clearance of activated effector T cells.

As a single agent, atezolizumab shows anti-tumor activity in both nonclinical models and in patients with cancer and is being investigated as a potential therapy in a wide variety of malignancies. Atezolizumab is being studied as a single agent in the advanced cancer and adjuvant therapy settings, as well as in combination with chemotherapy, targeted therapy, cancer immunotherapy and cellular therapy.

Atezolizumab is approved for the treatment of urothelial carcinoma, non-small cell lung cancer, small-cell lung cancer, triple-negative breast cancer, hepatocellular carcinoma, and melanoma. Atezolizumab is not approved for the treatment of PDAC or CRC.

### EXAMPLES

### Example 1. Initial Identification of TCR Candidates

In this example, a large pool of TCR candidates were identified and multiple layers of assays were used to progressively narrow the large pool of TCR candidates to the TCR sequences recited and claims in this application. In this process, the tools used for identifying KRAS antigen-specific T cells included exposing a tissue sample comprising T cells to antigen, activating T cell in a tissue sample by antigen, obtaining recombined nucleic acids from T cells of a tissue sample, isolating (or recovering, or sorting, or separating) activated T cells, sequencing recombined nucleic acids, forming clonotypes, and determining clonotypes of antigen-specific T cells.

### A. Tissue Samples:

Characterized PBMCs were collected and thawed, washed and either lysed with RLT plus buffer (Qiagen) for nucleic acid purification or cultured overnight in the presence of KRAS peptides (see below) to identify antigen-specific T cells.

### B. Antigen-Specific T Cell Assays, Flow Cytometry and Cell Sorting:

Antigen-specific cells were identified using a variety of assays: pentamer binding, cell surface marker upregulation (CD137, CD107) following short-term peptide incubation, and proliferation following relatively long-term peptide incubation. Pentamer-specific T cells were identified by incubating PBMCs with known KRAS antigenic peptides according to manufacturer's instructions.

Briefly, complete media containing 15% Fetal Bovine Serum (FBS), non-essential amino acids, glutamine and antibiotics was used for peptide incubations. Thawed PBMCs were washed and suspended at ~400,000 cells/well (96-well i-bottom plates) in complete media. Unconjugated antibodies directed against CD28 and CD49d were then added to the wells containing the suspended cells. Peptides derived from KRAS were added directly to the cell/antibody mixture. Following addition of peptides, cells were incubated at 37° C. for ~8 hours. Negative control incubations were prepared as outlined above without addition of peptides.

At the end of the incubation, cells were harvested from the culture and stained with antibodies for analysis and sorting by flow cytometry. For each CD8 antigen-specific assay (CD137 and CD107), fluorescently conjugated antibodies to the following cell surface markers were used for flow cytometry: CD8, CD3 and either CD137 or CD107a and CD107b. Cells were then washed and suspended in PBS containing FBS (2%) and 4′,6-diamidino-2-phenylindole (DAPI) for exclusion of non-viable cells. Carboxyfluorescein diacetate, succinimidyl ester (CFSE)-labeled PBMCs were incubated as outlined above for 6 days in the presence of peptide and antibodies directed against CD28 and CD49d. Antigen-specific CD8+ T cells were identified and sorted based on CFSE dilution at day 6. Cells were acquired and sorted using a FACSAria (BD Biosciences) instrument. Sorted antigen-specific (CD3⁺CD8⁺CMVpentamer⁺, CD3⁺CD8⁺CD137⁺, CD3⁺CD8⁺CD107a/b⁺, or CD8⁺CFSE^{low}) and non-antigen-specific (CD3⁺CD8⁺CD137⁻, CD3⁺CD8⁺CD107a/b⁻) cells were pelleted and lysed in RLT Plus buffer for nucleic acid isolation. Analysis of flow cytometry data files was performed.

C. TCR beta Amplification and Sequencing:

Isolated DNA from antigen-specific T cells was amplified using locus specific primer sets for TCR beta. This amplification reaction reproducibly amplified all possible RNA transcripts found in the sample containing the rearranged TCR beta locus regardless of which variable (V) segment and which common constant (C) region allele each rearranged molecule possessed, while appending the necessary sequences for cluster formation and sample indexing.

First stage primers were designed so as to allow for the amplification of all known alleles of the germline sequences, as described above and in the following; Faham et al, Blood, 120: 5173-5180 (2012). At the 5′ ends of the V segment primers, universal sequences complementary to second stage PCR primers were appended. Primers were optimized such that each possible V and C segment was amplified at a similar rate so as to minimally skew the repertoire frequency distribution during the amplification process. Specificity of the primers was, in contrast, not optimized as the primer sequences could be mapped and removed from the eventual sequence read. Thus, a given sequence may have been amplified by multiple primers.

In the second stage PCR, primers on the C side annealed to the C segment with a 5′ tail that contained the sequence primer and the P5 sequence used for cluster formation in the Illumina Genome Analyzer sequencer. Primers on the V side annealed to the V segment with a 5′ tail that contained the sequence primer and the P7 sequence used for cluster formation. For each sample, one pair of primers is used in the second stage. On the C side, it is always the same primer. On the V side, it is one of a set of primers which differs by a 6 base index. Specifically, the primers on the V side of the amplification constituted one of a set of primers, each of which had a 3′ region that annealed to the overhang sequence appended in the first reaction but which further contained one of multiple 6 base pair indices that allowed for sample multiplexing on the sequencer. Each of these primers further contained a 5′ tail with the adapter sequence used in an Illumina sequencer.

First stage PCR was carried out for 16 cycles. A second stage PCR was carried out for 22 cycles on 1/100 of the amplification products from the first stage PCR. Each sample contained a unique identifying tag. Samples were pooled and purified then cluster formation and sequencing in both directions was carried out per the manufacturer protocol (Illumina, Inc., La Jolla, Calif.). Specifically, three sequencing reactions were performed. First 115 bp were sequenced from the C side sufficient to sequence through the junctional sequence from C to V. At this point, the synthesized strand was denatured and washed off. A second sequencing primer was annealed that allowed the sample index to be sequenced for 6 cycles to identify the sample. At this point the reverse complement strand was generated per the Illumina protocol. A final sequencing read of 95 bp was obtained from the V-to-C direction providing ample sequence to map the V segment accurately. The sequencing data was then analyzed to determine the clonotype sequences, as described above.

### D. Clonotype Determination:

A clonotype was defined when at least 2 identical sequence reads were obtained. Briefly, after exclusion of low quality reads, sequence data were then analyzed to determine the clonotype sequences including mapping to germline V and J consensus sequences. First, the sample index sequences were used to identify which of the sequences originate from which of the pooled samples. Sequences whose index were not a perfect match to one of the indices used in a specific run were excluded. Next the forward read was used to map the J segment. Since all the sequences started from the same position of the J segments, all the J segments started at a predefined sequencing position. The first 25 bp of the J segments were used to map the J segment. Any read with more than 5 high quality mismatches to the known J segments was excluded from further analysis.

After J segment identification, V segments were mapped. The reverse read was used for this purpose. First, the V primer was mapped and excluded. Thereafter, the next 70 bases of the reverse read were mapped to the known V segments. Reads that did not map to J and V segments were excluded. The next step in mapping involved identifying the frame that related the forward and reverse reads and this allowed a continuous sequence from J to V to be constructed. This was done using the last 15 bases of the forward read which were reliably within the V segment regardless of NDN length. While these bases could be of relatively lower sequence quality as they were at the terminal end of a long read, they could be used to map within a single identified V segment in order to identify the position at which the two reads could be joined. Finally, the known V and J sequences to which the reads map were used to identify the point in the forward read at which the sequences at the junctions diverged from these mapped segments.

Following clonotype determination, relative frequencies of the clonotypes were analyzed in the unsorted, antigen-specific and non-antigen-specific populations. Clonotypes that are present in sample A but not in sample B (where frequencies in sample A and B are being compared) were represented to have the frequency of a clonotype with a single read in sample B. Therefore the frequency of the missing clonotype in a sample depends on the sequencing depth of a particular sample. In these cases where a clonotype is missing in a sample, because the frequency of a single read is assigned to these clonotypes, the observed frequency was overestimated. Thus, in the vast majority of these cases, the real clonotype frequency was likely to be overestimated. Clonotypes absent in both samples appear where the axes intersect. Clonotypes present in one sample but not the other however lie along either the x- or y-axis.

Clonotypes from the antigen-specific T cell analyses were selected based on three criteria. First, selected clonotypes had a frequency in sorted antigen-specific populations that was increased by at least 10-fold over the frequency in non-antigen-specific or unsorted cell populations. Second, these clonotypes were also present at lower frequencies in sorted, non-antigen specific cells compared to unsorted cells if greater than 1/100,000 in order to avoid sub-sampling error (Poisson noise) associated with very low frequency clonotypes in sorted samples. Third, because of the limited number of input antigen-specific cells after sorting (generally <30,000 cells), a greater than 20-cell equivalent threshold was applied based on the relatively low input number of cells in these samples. This minimum threshold enabled exclusion of clonotypes that appeared enriched in sorted antigen-specific samples but were due only to the presence of one or a few cells in the sample. For example, consider a sorted population of 10,000 pentamer+ cells (where the pentamer is a MHC-antigen complex) out of a sample with a million T cells. Pentamer+ cells are those cells on which peptide-HLA molecules have bound the TCR of T cells and represent those cells with the desired specificity to the peptide-HLA complex. If a single cell with a frequency of 1 per million in the unsorted sample is incidentally sorted in the pentamer+sample, its frequency in the sorted sample will be 1/10,000 and would appear to be 100-fold enriched in the pentamer+sample compared to the unsorted sample. To ameliorate this problem, a clonotype was required to represent at least 20 cells in the sorted pentamer+sample. Specifically, the log₁₀ frequency threshold required in the pentamer+sample was calculated as log₁₀(1/(n/20)), where n is the number of input sorted cells for that sample as determined by flow cytometry.

Those sequences meeting the three criteria outlined above were classified as antigen-specific T cell clonotypes. Antigen-specific T cells so identified were then further characterized for KRAS peptide activation, cytotoxicity, cytokine secretion and avidity as further described below. Alpha and beta chain matches for the T cells with the most desirable characteristics were also performed.

30,350 TCRs were identified specific to KRAS G12X using this method (by G12X, is meant a mutation with any other amino acid at the G12 position). The KRAS-p.G12 mutations that were screened for included G12A, G12C, G12D, G12R, G12S and G12V.

### Example 2. Identifying Optimal Clinical TCR Candidates

Naïve CD8+ T cells isolated from healthy donors were expanded ~14 days and then subjected to MIRA (Multiplexed Identification of Receptors for Antigen) using a panel of 376 selected neoantigenic epitopes. TCR beta chain sequences were assigned to neoantigens in silico via MIRA, while their paired TCR alpha chain sequences were discovered in parallel using pairSeq. TCR alpha/beta pairs (henceforward ‘TCRs’) deemed specific for mutant KRAS epitopes by MIRA analysis were selected from HLA-A11:01-positive donors for gene synthesis and further investigation. TCRs were subjected first to a cellular reactivity assay: briefly, TCR constructs were electroporated into a modified Jurkat-based cell line, which were then exposed to monoallelic HLA-A11:01-positive antigen-presenting cells bearing either the KRAS-p.G12D peptide VVVGADGVGK (SEQ ID NO: 1) or the wild type KRAS peptide VVVGAGGVGK (SEQ ID NO: 102). 73 TCRs showing specificity for the mutant peptide but no reactivity toward the wild type peptide were selected for further analysis (the remainder of the TCRs being typically specific for the KRAS-p.G12V peptide VVVGAVGVGK (SEQ ID NO: 103), and/or for other KRAS-derived peptide/HLA combinations). These 73 TCRs were further subject to a peptide titration experiment using the same Jurkat-based assay: 18 TCRs with the most optimal dose-responsiveness (e.g. those capable of responding to the lowest peptide concentrations) were further selected for downstream assays as described in Example 3. Briefly, these TCRs were electroporated into expanded primary CD8+human T cells, then these T cells were exposed to a series of KRAS-transgene-transfected target cells and naturally KRAS-p.G12D mutant tumor cell lines. Among these, the EE209_2 TCR (SEQ ID Nos: 12 and 14) was selected for its superior ability to recognize KRAS-p.G12D-transfected cell lines, as well as naturally KRAS-p.G12D-expressing tumor cell lines. Additionally, at this time, KRAS G12D-specific TCRs PD6_KRASmut_9 (SEQ ID Nos: 44 and 45), PD6_KRASmut_22 (SEQ ID Nos: 34 and 35), PD22_KRASmutwt_1 (SEQ ID Nos: 54 and 55), PD45_KRASmutwt_4 (SEQ ID Nos: 64 and 65), EE208_KRAS_1 (SEQ ID Nos: 74 and 75), EE184_KRAS_8a (SEQ ID Nos: 24 and 25), OX92_KRAS_36 (SEQ ID Nos: 84 and 85), and EE231_KRAS_52 (SEQ ID Nos: 94 and 95) were also selected for their properties.

### Example 3. Functional Assessment of Select KRAS-G12D Specific TCRs

Cytolysis, cell activation, and cytokine secretion assays were performed to assess the antigen-specific killing capacities of select KRAS-G12D specific TCRs.

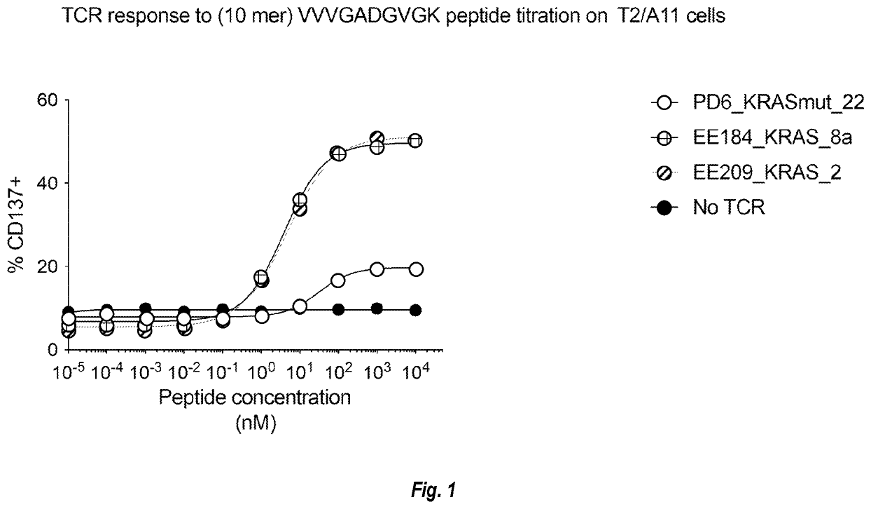

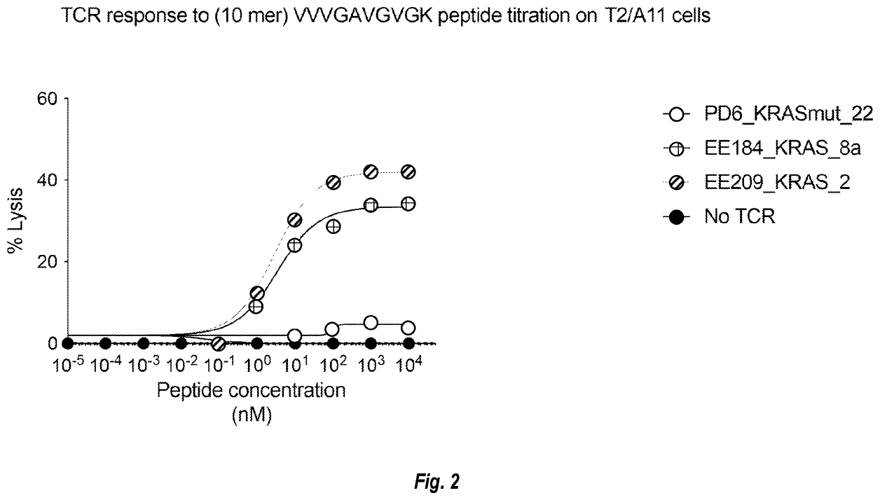

### FIGS. 1 and 2: T Cell Activation and Killing of Peptide Loaded Targets (% CD137 and % Lysis).

The T2 cell line (deficient in the TAP protein) was modified using a viral vector expressing HLA-A*11:01. These T2/A*11:01 cells were then incubated with increasing concentrations of KRAS 10-mer mutant peptide (VVVGADGVGK) (SEQ ID NO: 1) for 30 mins at room temperature, after which excess peptide was washed away with R10 media (10% FBS+RPMI). Concurrently, a separate batch of unmodified T2 cells (T2) was washed with PBS, then incubated with 1× CellTrace Far Red (ThermoFisher C34564) for 30 mins. After the incubation period, the excess CellTrace stain was washed off with R10 media. T2/A*11:01 peptide pulsed cells and CellTrace labeled T2 cells were then mixed at a 1:1 ratio. In essence, each mixture contained peptide pulsed T2/A*11:01 cells presenting a specific concentration of peptide as well as CellTrace labeled (non-peptide pulsed) T2 cells in equal amounts.

Polyclonally expanded human CD8+ T cells were transfected with no TCR or the indicated KRAS-G12D specific TCRs, then incubated with the mixture of peptide-pulsed/cell-trace labeled (T2/A*11:01+T2) cells. After 16 hours of incubation, these polyclonal human CD8 T cells were evaluated by flow cytometry for activation by evaluating the percentage of CD137 positive cells. FIG. 1. The cytolysis potential of each TCR was also evaluated by comparing the percentage of viable peptide pulsed T2/A*11:01 between TCR and No TCR control. The presence of CellTrace labeled cells with no peptide presentation acts as a control to allow the calculation of specific lysis due to the presence of the mutant KRAS peptide. FIG. 2. The data here show that compared to the No TCR control, all TCRs used were activated in the presence of peptide in a dose dependent manner. FIG. 1. A higher percentage of cells with EE184_KRAS_8a and EE209 KRAS 2 TCRs were activated compared to those cells with PD6_KRASmut_22, and were activated at lower peptide doses. Likewise, lysis was observed with the most active TCRs (EE184 KRAS 8a and EE209_KRAS_2) in a dose dependent manner. FIG. 2.

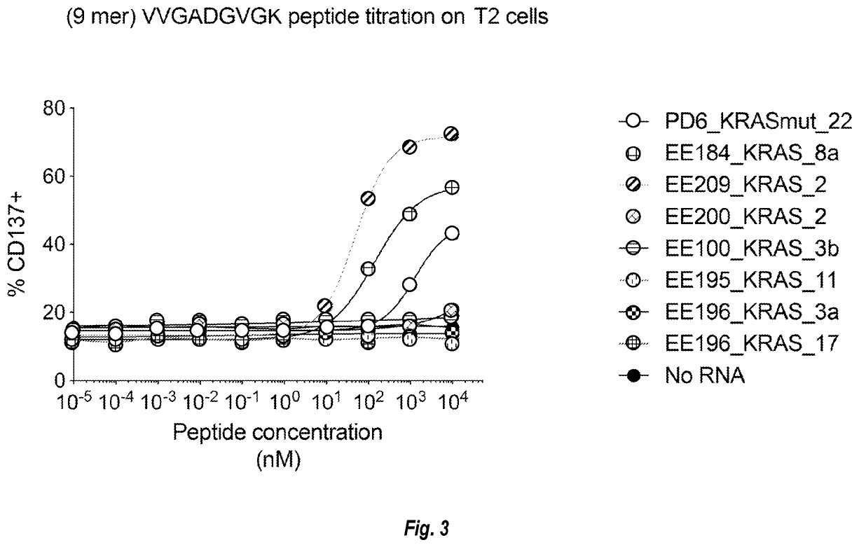

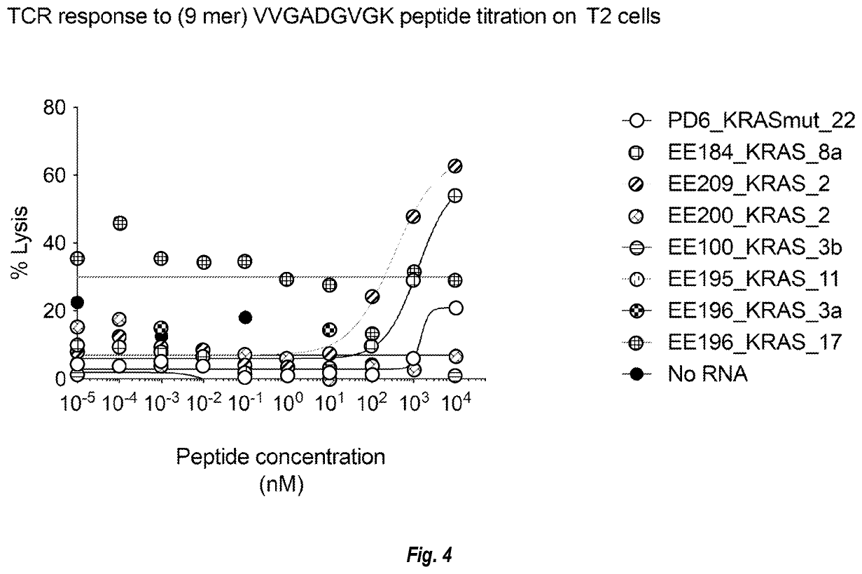

FIGS. 3 and 4: T cell activation and killing of peptide loaded targets (% CD137 and % Lysis). The T2 cell line (deficient in the TAP protein) was modified using a viral vector expressing HLA-A*11:01. These T2/A*11:01 cells were then incubated with increasing concentrations of KRAS 9-mer mutant peptide (VVGADGVGK) (SEQ ID NO: 2) for 30 mins at room temperature, after which excess peptide was washed away with R10 media (10% FBS+RPMI). Concurrently, unmodified K562 cells (K562 plain) were washed with PBS, then incubated with 1× CellTrace Far Red (ThermoFisher C34564) for 30 mins. After the incubation period, the excess CellTrace stain was washed off with R10 media. Peptide pulsed T2/A*11:01 cells and CellTrace labeled K562 plain cells were then mixed at a 1:1 ratio. In essence, each mixture contained peptide pulsed T2/A*11:01 cells presenting a specific concentration of peptide as well as CellTrace labeled (non-peptide pulsed) K562 plain cells in equal amounts.

After 16 hours of incubation, these polyclonal human CD8+ T cells were evaluated by flow cytometry for activation by evaluating the percentage of CD137 positive cells as shown in FIG. 3. The cytolysis potential of each TCR was also evaluated by comparing the percentage of viable peptide pulsed T2/A*11:01 between TCR and No TCR control. The presence of CellTrace labeled cells with no peptide presentation acts as a control to allow the calculation of specific lysis due to the presence of the mutant KRAS peptide as shown in FIG. 4. The data here show that compared to the No TCR control, PD6_KRASmut_22, EE184_KRAS 8a and EE209_KRAS_2 were activated in the presence of peptide in a dose dependent manner. FIG. 3. A higher percentage of cells with EE184_KRAS_8a and EE209_KRAS_2 TCRs were activated compared to those cells with PD6_KRASmut_22, and were activated at lower peptide doses. Likewise, lysis was observed with the most active TCRs (EE184_KRAS_8a, EE209 KRAS 2 and to some extent, PD6_KRASmut_22) in a dose dependent manner. FIG. 4. None of the other TCRs tested (EE200_KRAS_2, EE100_KRAS_3b, EE195_KRAS_11, EE196_KRAS 3a and EE196_KRAS 17) were activated or exhibited lysis due to T2/A11 peptide presentation.

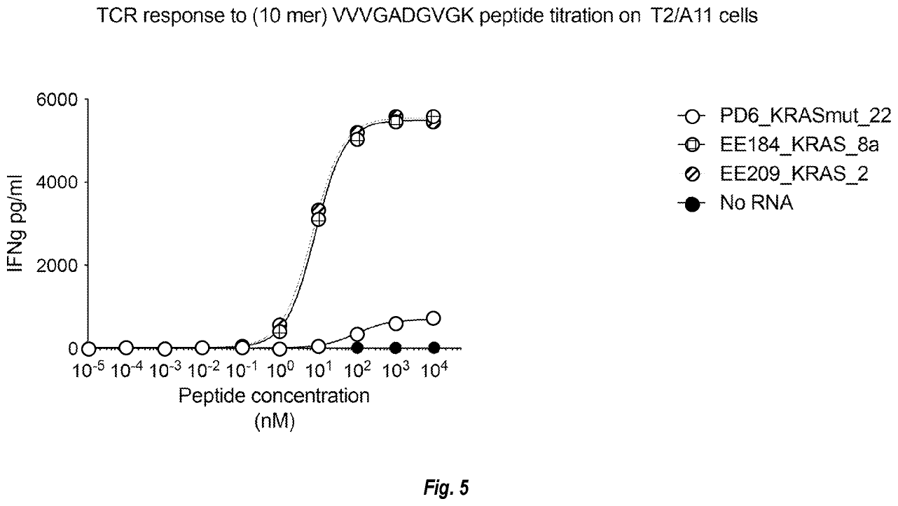

FIG. 5: Cytokine secretion from activated T cells incubated with peptide loaded targets. For evaluation of cytokine secretion, supernatants from T cells expressing KRAS-specific TCRs after incubation with the KRAS 10-mer mutant peptide (VVVGADGVGK) (SEQ ID NO: 1) peptide pulsed T2/A*11:01+CellTrace T2 mixture were assessed. IFNγ secretion was assessed using a Human IFN-γ Flex set cytokine bead array kit from BD Biosciences (CBA, Cat #560111). The data shows that T cells secreted IFNγ in a mutant-KRAS-specific and dose dependent manner. PD6_KRASmut_22 had the lowest levels of secretion while EE184_KRAS_8a and EE209_KRAS_2 had comparable levels of IFNg secretion and were capable of IFNγ secretion at lower doses of peptide than PD6_KRASmut_22.

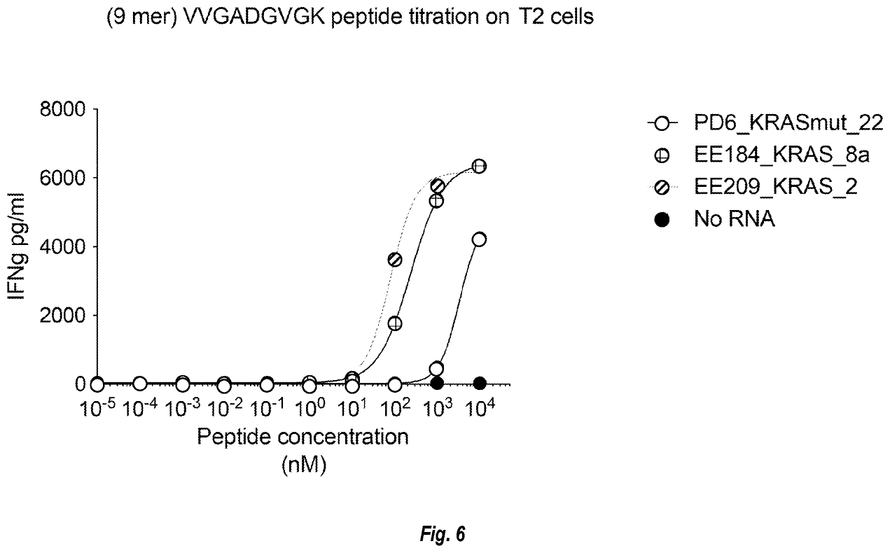

FIG. 6: Cytokine secretion from activated T cells incubated with peptide loaded targets. The T2 cell line (deficient in the TAP protein) was modified using a viral vector expressing HLA-A*11:01. These T2/A*11:01 cells were then incubated with increasing concentrations of KRAS 9-mer mutant peptide (VVGADGVGK) (SEQ ID NO: 2) for 30 mins at room temperature, after which excess peptide was washed away with R10 media (10% FBS+RPMI). Concurrently, unmodified K562 cells (K562 plain) were washed with PBS, then incubated with 1× CellTrace Far Red (ThermoFisher C34564) for 30 mins. After the incubation period, the excess CellTrace stain was washed off with R10 media. Peptide pulsed T2/A*11:01 cells and CellTrace labeled K562 plain cells were then mixed at a 1:1 ratio. In essence, each mixture contained peptide pulsed T2/A*11:01 cells presenting a specific concentration of peptide as well as CellTrace labeled (non-peptide pulsed) K562 plain cells in equal amounts.

Polyclonally expanded human CD8+ T cells were transfected with no TCR or the indicated KRAS-G12D specific TCRs, then incubated with the mixture of peptide-pulsed/cell-trace labeled (T2/A*11:01+K562 plain) cells. After 16 hours of incubation, supernatants from T cells expressing KRAS-specific TCRs after incubation with the aforementioned peptide pulsed T2/A*11:01+CellTrace T2 mixture were assessed as shown in FIG. 6. IFNγ secretion was assessed using a Human IFN-γ Flex set cytokine bead array kit from BD Biosciences (CBA, Cat #560111). The data shows that TCRs secreted IFNγ in a mutant-KRAS-specific and dose dependent manner. PD6_KRASmut 22 had the lowest levels of secretion while EE184 KRAS 8a and EE209_KRAS_2 had comparable levels of IFNg secretion and were capable of IFNγ secretion at lower doses of peptide than PD6_KRASmut_22. FIG. 6.

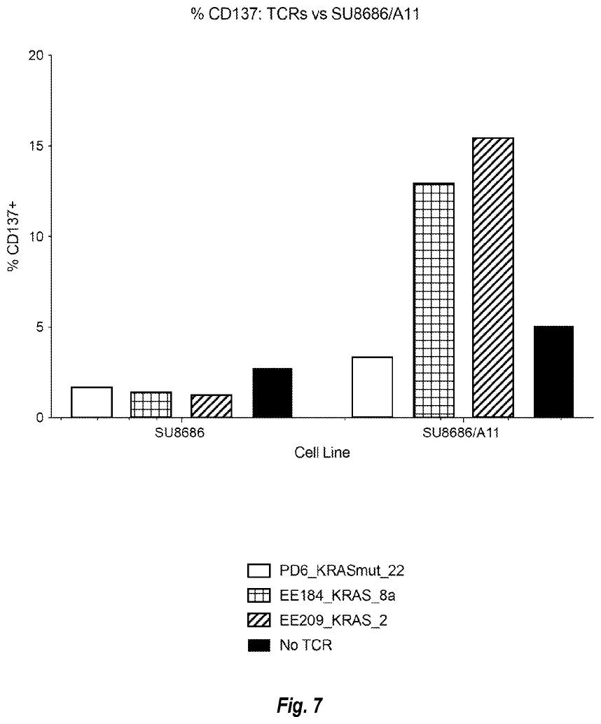

FIGS. 7, 8, and 9: T cell activation in response to cell lines harboring the KRAS-p.G12D mutation (−/+HLA A*11:01). The following tumor cell lines from a variety of cancer types expressing the KRAS-p.G12D mutation were utilized: SNU1 (gastric carcinoma), SU8686 (pancreatic carcinoma), HuCCT1 (liver (cholangio) carcinoma). Additionally, the following tumor cell lines without the KRAS G12D mutation were also utilized: NCIH1373 (lung carcinoma), RKN (ovarian carcinoma), CFPAC1 (pancreatic carcinoma), NCIH727 (lung carcinoma) and SW527 (colorectal carcinoma). All the aforementioned cell lines with the exception of HuCCT1 (which naturally expresses HLA-A11*01) were transduced with a viral vector expressing HLA A*11:01.

Polyclonal human CD8 T cells were transfected with the indicated TCRs and incubated for 16 hours with the tumor cell lines. Response of TCRs to the tumor cell lines with or without HLA A*11:01 expression was evaluated by flow cytometry by measuring activation via percentage of CD137 positive cells. None of the TCRs were activated in the absence of HLA A*11:01 on the SU8686 tumor cell line. But EE184 KRAS 8a and EE209 KRAS 2 were activated when incubated with SU8686/A*11:01 cells. FIG. 7.

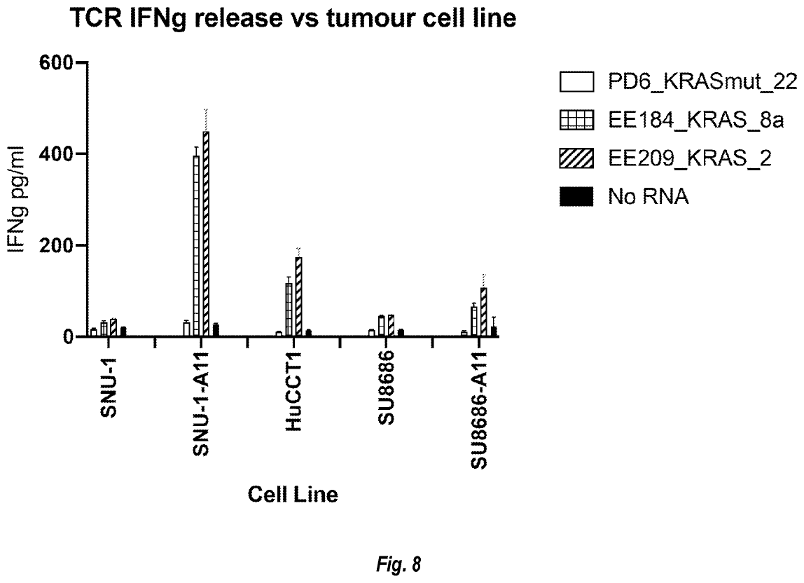

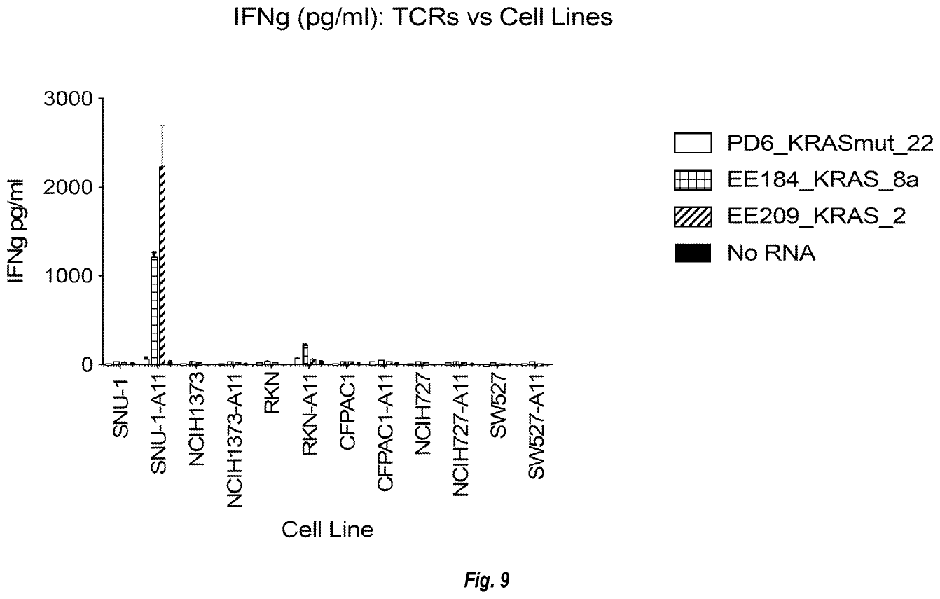

FIGS. 8 and 9: Cytokine secretion from expanded panel of cell lines with KRAS-p.G12D mutation (−/+A*11:01). For the cytokine evaluation, supernatants were assessed for IFNγ secretion using a Human IFN-γ Flex set cytokine bead array kit from BD Biosciences (CBA, Cat #560111). IFNγ was assessed for each TCR after incubation with the tumor cell lines with or without HLA A*11:01 expression. As shown, EE184 KRAS 8a and EE209 KRAS 2 secreted IFNγ in response to tumor cell lines that were positive for both KRAS G12D and HLA A*11:01. FIG. 8. None of the TCRs responded to any cell lines that did not express KRAS G12D, even if the cell lines expressed HLA A*11:01. FIG. 9.

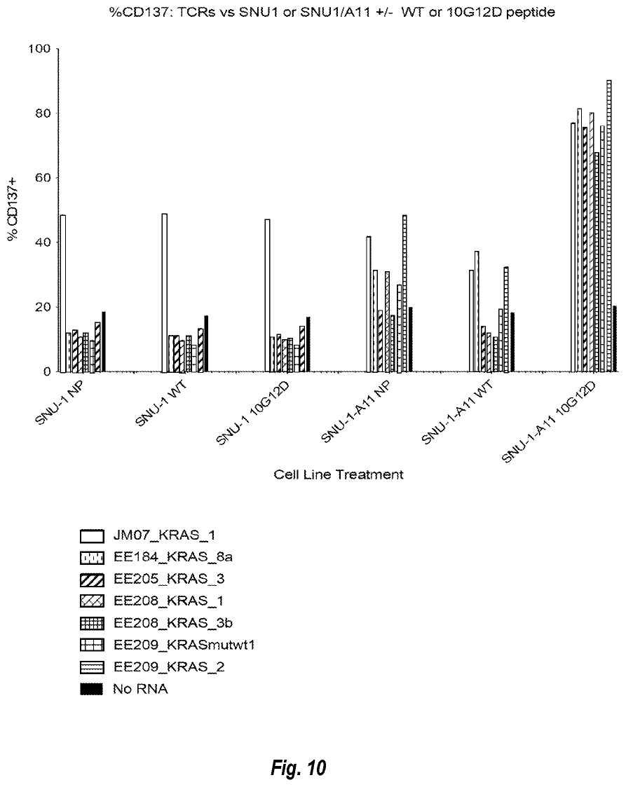

FIG. 10: T cell activation in response to cell lines harboring the KRAS-p.G12D mutation (−/+HLA A*11:01) in addition to treatment with 10-mer peptides. The gastric carcinoma cell line: SNU1 was transduced with a viral vector expressing HLA A*11:01. SNU1 and SNU1/A*11:01 cells were then incubated for 30 mins with 10-mer wildtype KRAS peptide (VVVGAGGVGK) (SEQ ID NO: 102), 10-mer mutant KRAS G12D peptide (VVVGADGVGK) (SEQ ID NO: 1) or no peptide was added. After the incubation period, excess peptide was washed off with R10 media (10% FBS+RPMI).

Polyclonal human CD8+ T cells were transfected with the indicated TCRs and incubated for 16 hours with the tumor cell lines with no peptides (SNU-1 NP or SNU-1-A11 NP) or with wildtype peptides (SNU-1 WT or SNU-1-A11 WT) or with mutant peptides (SNU-1 9G12D or SNU-1-A11 9G12D). Response of TCRs to the aforementioned tumor cell line preparations with or without HLA A*11:01 expression was evaluated by flow cytometry by measuring activation via percentage of CD137 positive cells.

JM07_KRAS_1 was activated both in the presence or absence of HLA A*11:01 expression as shown in FIG. 10. EE184_KRAS_8a, EE209_KRAS_2, EE208 KRAS_1, EE209_KRASmutwt1, EE205_KRAS_3 and EE208_KRAS_3b were activated only in the presence of HLA A*11:01. FIG. 10. The addition of 10G12D mutant peptide increased the activation of all TCRs tested compared to no peptide or WT peptide treatments.

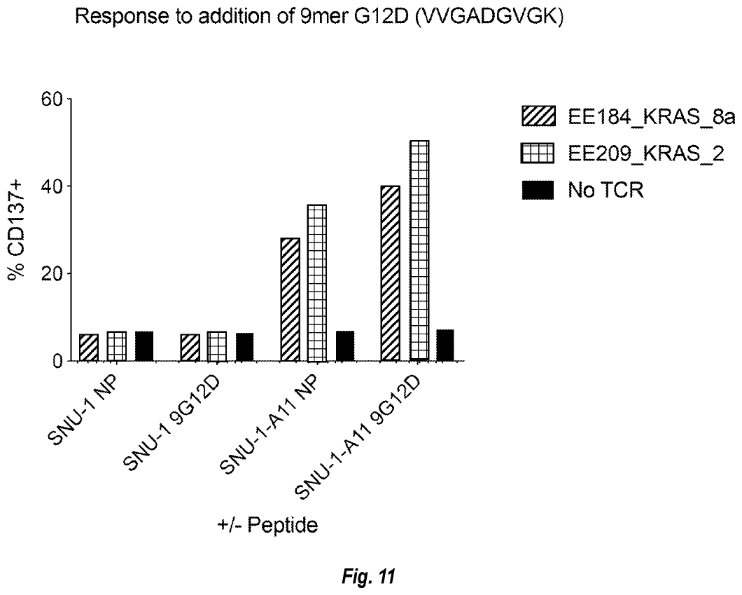

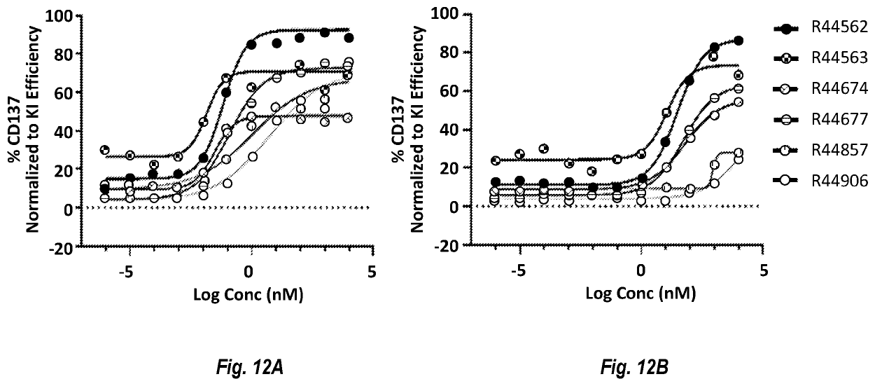

FIG. 11: T cell activation in response to cell lines harboring the KRAS-p.G12D mutation (−/+HLA A*11:01) in addition to treatment with 9-mer peptides. The gastric carcinoma cell line SNU1 was transduced with a viral vector expressing HLA A*11:01. SNU1 and SNU1/A*11:01 cells were then incubated for 30 mins with 9-mer wildtype KRAS peptide (VVGAGGVGK) (SEQ ID NO: 104), 9-mer mutant G12D KRAS peptide (VVGADGVGK) (SEQ ID NO: 2) or no peptide was added. After the incubation period, excess peptide was washed off with R10 media (10% FBS+RPMI).

Polyclonal human CD8+ T cells were transfected with the indicated TCRs and incubated for 16 hours with the tumor cell lines with no peptides (SNU-1 NP or SNU-1-A11 NP) or with wildtype peptides (SNU-1 WT or SNU-1-A11 WT) or with mutant peptides (SNU-1 10G12D or SNU-1-A11 10G12D). Response of TCRs to the aforementioned tumor cell line preparations with or without HLA A*11:01 expression was evaluated by flow cytometry by measuring activation via percentage of CD137 positive cells.

EE184_KRAS_8a and EE209_KRAS_2 were activated only in the presence of HLA A*11:01 as shown in FIG. 11. The addition of 9G12D mutant peptide also increased the activation of EE184_KRAS_8a and EE209_KRAS_2 cells compared to no peptide or WT peptide treatments. FIG. 11.

### Example 4. Functional Avidity Assessment of EE209_2 TCR Using HLA-A*11:01+B-Cells

Additional cytolysis, cell activation, and cytokine secretion assays were performed to functionally assess the EE209_2 TCR (SEQ ID NOs: 12 and 13).

### A. Flow Cytometry to Measure Specific Lysis and T Cell Activation

HLA-A*11:01+ B cells obtained from the International Histocompatibility Working Group (IHW01109) were suspended at a cell density of 1 million cells per mL in B cell media (RPMI with 15% FBS+1× glutamine+1×NEAA+1× sodium pyruvate) and pulsed with varying concentrations of 9-mer and 10-mer KRAS G12D mutant and wildtype KRAS peptides (covering 11 points of 10-fold dilutions from 10 μM to 1 fM). Target B cells were incubated with peptides for at least 2 hours at 37° C. and 5% CO₂ in a humidified incubator. After peptide pulsing, target B cells were washed twice with B cell media, counted using the Vi-Cell Blu, and resuspended at 1 million cells per mL. 50 μl of this dilution was added to a 96-well round bottom plate (Corning, Cat. #3799) for 50,000 cells per well.

HLA-A*11:01-negative B cells from the International Histocompatibility Working Group (IHW01117) were included as a non-target B cell population in the co-cultures and used to calculate specific lysis of the target B cell population. Non-target B cells were suspended at 1 million cells per mL and labeled with 1 μM of Cell-trace violet (CTV) dye for 10 minutes at 37° C. and 5% CO₂. Following the 10-minute incubation, non-target B cells were washed twice with B cell media, incubated at 37° C. and 5% CO₂ for at least 1 hour, and washed one final time to remove excess dye. CTV-labeled cells were added to the wells containing the HLA-A*11:01+target B cells at 1:1 ratio (50,000 cells each).

TCR KO T cells and T cells expressing the KRAS G12D-specific EE209_2 TCR were thawed and rested overnight in T cell media supplemented with 25 ng/ml IL-7 and 50 ng/ml IL-15. The following day, T cells were harvested, washed twice with T cell media, and counted using the Vi-Cell Blu cell viability reader. 50,000 viable T cells were added to the mixture of target and non-target B cells, resulting in an E:T ratio of 1:1 for T cells to peptide-pulsed target B cells.

T cells and B cells were co-cultured for 18-20 hours at 37° C. and 5% CO₂ in a humidified incubator. After incubation, supernatants and cells were harvested using a Tecan 480 liquid handler. Supernatants were collected in a 384-well V-bottom plate (Greiner Bio, Catalog #784201) using a Tecan 480 liquid handler, then frozen at −80° C.

Cells harvested from the co-cultures were stained for the following markers for flow cytometric analysis: Live/Dead viability dye APC 780 (Invitrogen, Catalog #65-0865-14), CD19 PE (Biologend, Catalog #392506), CD20 APC (Biolegend, Catalog #302310), CD8 BUV395 (BD Biosciences, Catalog #563795), and CD137 brilliant violet 605 (Biolegend, Catalog #309822). Cell-trace violet signal was used to identify non-target B cells. B cell and T cell co-cultures were washed twice with PBS (1200 RPM for 5 minutes at room temperature), and then stained with the viability dye for 30 minutes on ice, protected from light. Cells were washed twice using FACS buffer (2% Bovine Serum Albumin in PBS), and then incubated in 25 μl of Fc block diluted in FACS buffer for 5 minutes on ice. An equal volume of the antibody cocktail listed in Table 2 was added to the cells in Fc block, and cells were incubated for an additional 30 minutes on ice, protected from light. Cells were washed twice with FACS buffer, then fixed with 1% paraformaldehyde. Cells were analyzed on a Cytoflex LX cytometer (Beckman Coulter) and data were analyzed in FlowJo software. CD137 positive T cells were identified and gated from CD8 positive T cells from single live lymphocytes, and target and non-target B cells were identified and gated from CD8 negative and CD19 positive from single live lymphocytes. Upregulation of CD137 in response to 10-mer G12D and 9-mer G12D peptide by CD8+ T cells expressing the KRAS G12D-specific EE209_2 TCR is shown in FIGS. 12(A) and 12(B), respectively.

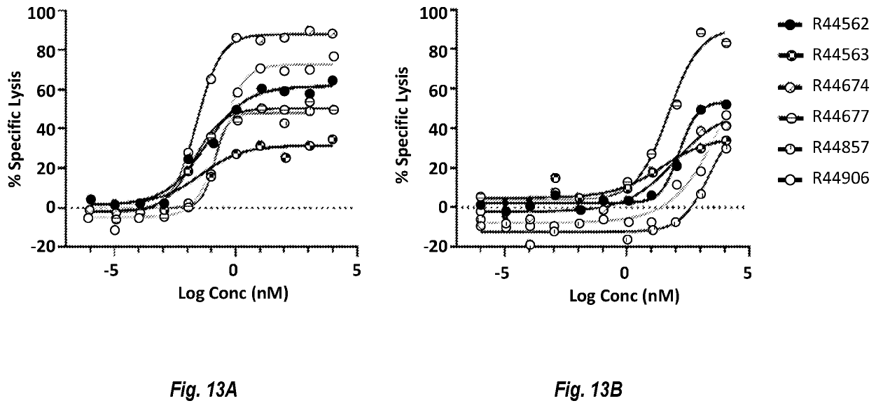

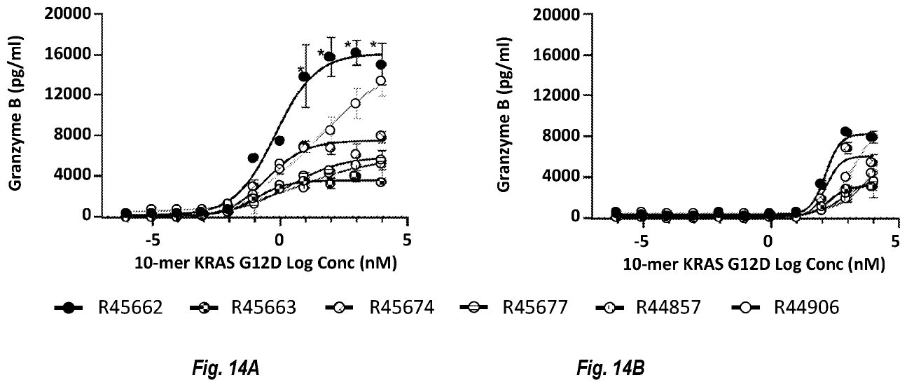

To calculate the percentage of specific lysis, the ratio of target to non-target B cells was calculated and normalized using Equation 1 shown below. Specific lysis of HLA-A*11:01+target B cells by CD8+ T cells expressing the KRAS G12D-specific EE209_2 TCR in the presence of 10-mer or 9-mer KRAS G12D mutant peptides is shown in FIGS. 13A and 13B. EC₅₀ values were calculated using four parameter sigmoidal fit with Graphpad Prism 8.4.3 software.

$$\% Specific Lysis = ( ( 1 - target to non - target B cell ratio in no peptide control condition ) / target to non - target B cell ratio in experim ental condition ) \times 100 \qquad Equation 1$$

TABLE 2
Antibodies used for analysis of T cell and B cell co-cultures

| Antibody | Fluorophore | Clone | Company | Cat# |
|---|---|---|---|---|
| Viability Dye | APCCy7 | — | Invitrogen | 65-0865-14 |
| | | | (Waltham, MA) | |
| Proliferation | Cell-Trace | — | Invitrogen | C34557 |
| Dye | Violet | | (Waltham, MA) | |
| Anti-CD19 | PE | 4G7 | BioLegend | 392506 |
| | | | (San Diego, CA) | |
| Anti-CD20 | APC | 2H7 | BioLegend | 302310 |
| | | | (San Diego, CA) | |
| CD8 | BUV395 | RPA-T8 | BD Biosciences | 563795 |
| | | | (Franklin | |
| | | | Lakes, NJ) | |
| CD137 | BV-605 | 4B4-1 | BioLegend | 309804 |
| | | | (San Diego, CA) | |

### B. Luminex for Cytokine Quantification

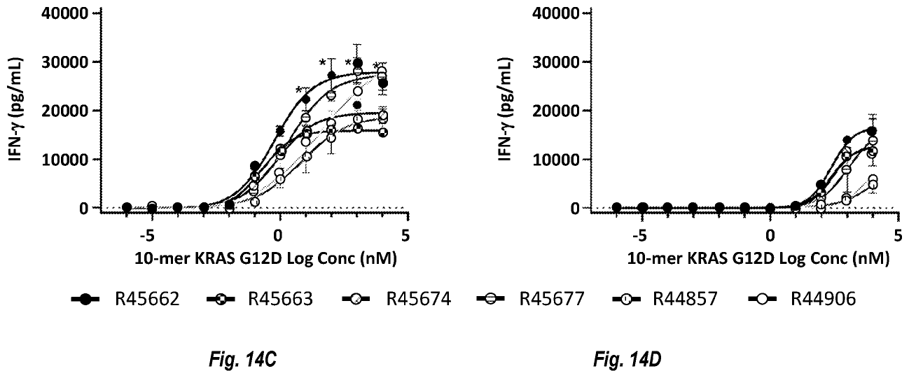

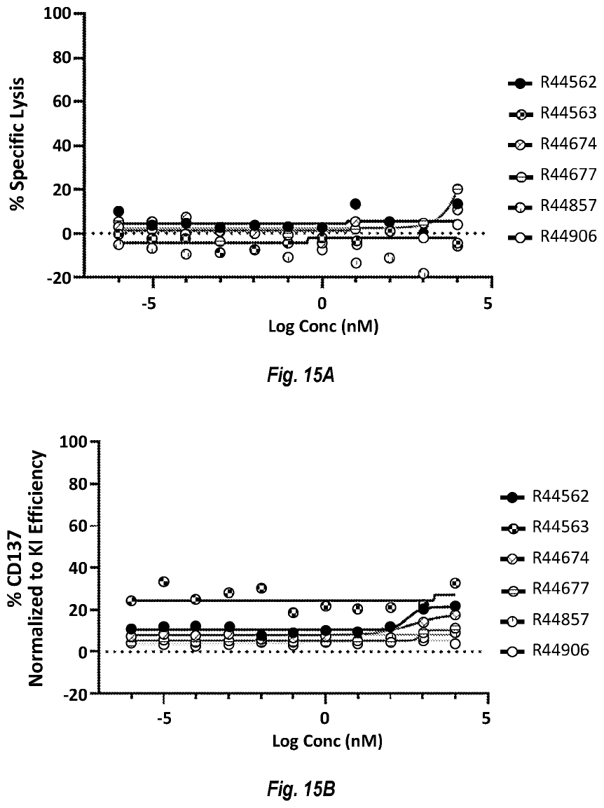

Supernatants were analyzed for granzyme B and IFNγ using Luminex magnetic beads from the Human Luminex Discovery Assay kit (R&D Systems; Minneapolis, MN; Catalog No. LXSAHM-06) and a Luminex FLEXMAP 3D® reader (Austin, TX). Standards were prepared according to the manufacturer's protocol and diluted supernatants (1:2 or 1:10 dilution) were added to wells containing 6 μL of Luminex magnetic beads and incubated at 4° C. overnight with shaking (600 rpm). The next day, plates were warmed to room temperature, spun down for 2 minutes at 1200 rpm, and washed twice using a Blue® Washer magnetic carrier (BlueCatBio; Concord, MA). Six microliters of a 1:10 dilution of biotin antibody was added to each well and the plates were incubated using the following protocol: 1 minute of shaking using a Genie® microplate mixer set at 2.5, followed by 1 hour of shaking using an ORBI-SHAKER™ plate shaker (Benchmark Scientific; Sayreville, NJ) set at 600 rpm. After incubation, plates were washed twice using a Blu® Washer magnetic carrier and then incubated for an additional 30 minutes after the addition of 6 μL streptavidin-PE. Beads were washed twice, 40 μL of 1x wash buffer was added, and the samples were read using a Luminex FLEXMAP 3D® reader. Median fluorescence intensity values were exported to Genedata software (Basel, Switzerland) for quantitation using a standard curve for granzyme B and IFNγ. Calculated concentrations were visualized using TIBCO Spotfire® (Palo Alto, CA). EC₅₀ values were calculated using four parameter sigmoidal fit with Graphpad Prism 8.4.3 software. The lower limit of detection (LLOD) for IFN-γ was 184.5 pg/ml for donors R44906 and R44857, and 104.6 μg/mL for donors R44562, R44563, R45674, and R45677. The lower limit of detection (LLOD) for Granzyme B was 42.6 μg/ml for donors R44906 and R44857, and 36.3 μg/mL for donors R44562, R44563, R45674, and R45677. For data points where the measured values fell below the LLOD of the assay, the LLOD value was graphed and used to calculate EC₅₀ values. Production of (A, B) Granzyme B and (C, D) IFN-γ by EE209_2 TCR-expressing CD8+ T cells is shown in FIG. 14.

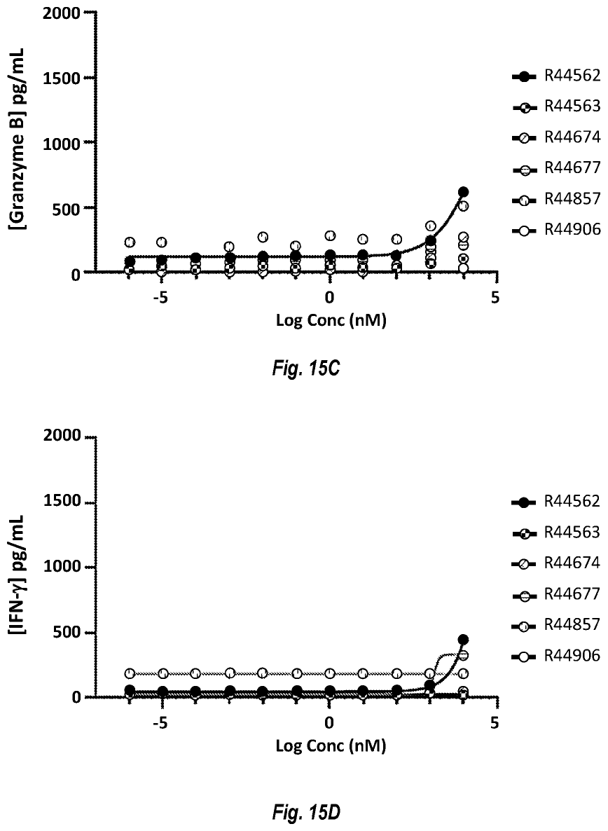

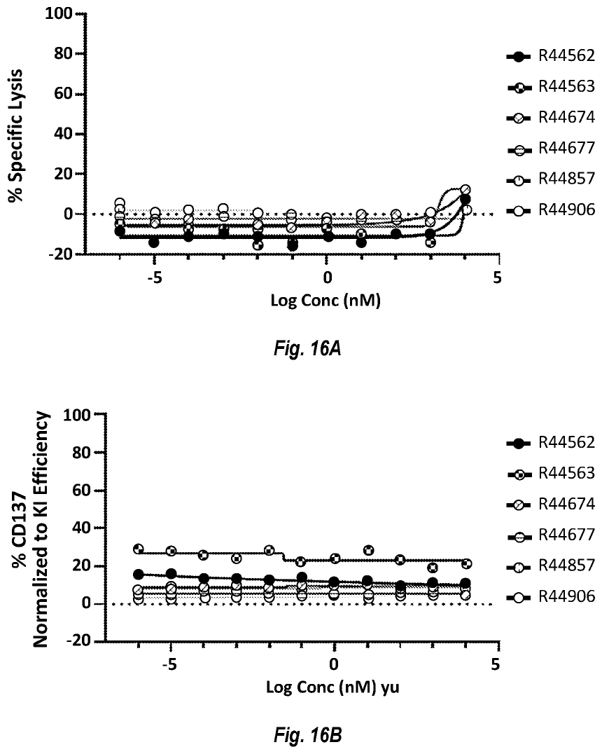

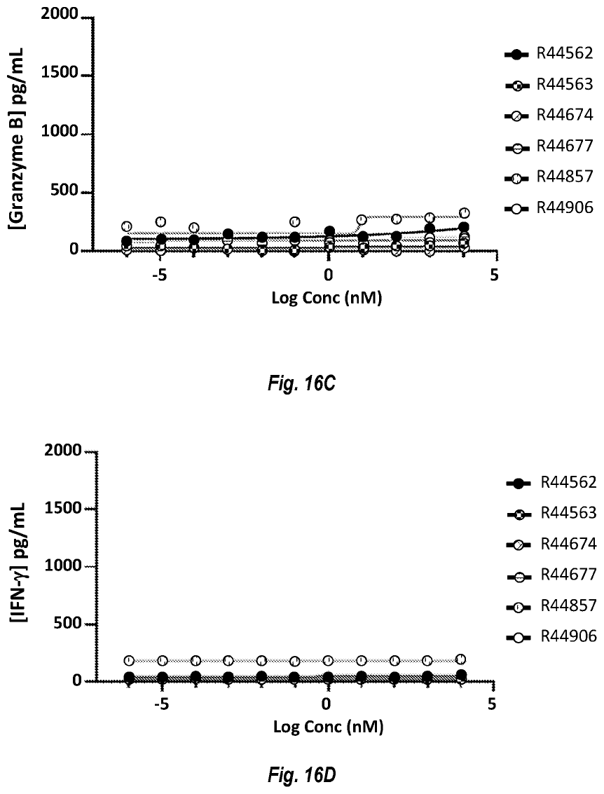

The activity, including A) target cell lysis, (B) upregulation of CD137, normalized to TCR knock-in efficiency, (C) Granzyme B production, and (D) IFN-γ production, of CD8+ T cells expressing EE209_2 TCR against the 10-mer wildtype KRAS peptide and 9-mer wildtype KRAS peptide is shown in FIG. 15(A)-(D) and FIG. 16(A)-(D), respectively.

### C. Conclusions

CD8+ T cells expressing the KRAS G12D-specific EE209_2 TCR were activated by both the 10-mer and 9-mer G12D peptides, but showed greater sensitivity to the 10-mer G12D peptide for specific target cell lysis, upregulation of CD137, granzyme B production, and IFN-g production. In addition, the KRAS G12D-specific EE209_2 TCR was specific for the mutant KRAS G12D peptide and was not activated by either the 10-mer or 9-mer wildtype KRAS peptide.

### Example 5. Assessment of EE209_2 TCR Using HLA-A*11:01⁺ KRAS G12D-Mutant Tumor Cell Lines

Additional cytolysis, cell activation, and cytokine secretion assays were performed to functionally assess the EE209_2 TCR (SEQ ID NOs: 12 and 13). The following tumor cell lines were used:

TABLE 3
Tumor Cell Lines

| Cell Line | Tissue | KRAS | HLA-A*11:01 |
|---|---|---|---|
| CL-40 | Colon | G12D | Endogenous |
| HuCCT1 | Liver | G12D | Endogenous |
| Panc 05.04 | Pancreas | G12D | Endogenous |
| SNU-601 | Stomach | G12D | Endogenous |
| PANC-1 | Pancreas | G12D | Endogenous |
| PK-59-A11 | Pancreas | G12D | Lentiviral |
| | | | expression |
| Panc 02.03-A11 | Pancreas | G12D | Lentiviral |
| | | | expression |

### A. T Cell-Dependent Cellular Cytotoxicity Assay: Impedance Assay

Prior to adding tumor cells to 96-well E-Plate VIEW plates (Agilent; Santa Clara, CA; Catalog No. 300601030), 50 mL of T cell media (RPMI with 10% FBS, 1x glutamine, 1×NEAA, 1× sodium pyruvate, 1×BME, and 1×HEPES) was added to each well to determine background measurements using an Agilent xCELLigence RTCA MP instrument (located in a humidified incubator at 37° C. with 5% CO2). After the background assessment was completed, the E-plates were removed from the xCELLigence RTCA MP instrument and 1.5×10⁴ tumor cells were added to each well. Tumor cells were allowed to settle for 30 minutes at room temperature before the E-plates were returned to the xCELLigence RTCA MP instrument to initiate the impedance/cell index measurements.

On the day of tumor cell seeding, TCR knock-out and KRAS G12D TCR CD8+ T cells were thawed and cultured overnight in media supplemented with 25 ng/ml hIL-7 and 50 ng/ml hIL-15. The following day, TCR knock-out and KRAS G12D TCR T cells were harvested, washed twice with T cell media, counted using a Vi-CELL BLU cell viability reader (Beckman Coulter; Indianapolis, IN), and resuspended in T cell media.

Once the T cells were prepared, E-plates were removed from the xCELLigence RTCA MP instrument and 10 mM of 10-mer KRAS G12D peptide was added to tumor cells to generate peptide-pulsed control conditions. Pulsing the tumor lines with the 10-mer KRAS G12D peptide served as a positive control for T cell activation in conditions where we have provided high amounts of the peptide. Although the tumor lines are endogenous for KRAS G12D, providing exogenous peptide and assessing T cell activation was used to establish that the tumor cells all express sufficient levels of the KRAS G12D peptide to activate T cells.

TCR knock-out and KRAS G12D TCR T cells were added to tumor cells at varying effector-to-target (E:T) ratios (1:1, 3:1, and 9:1). All manipulations were conducted with the E-plate on a 37° C. hot plate to minimize effects of temperature change on cell morphology. Following the addition of the T cells, E-plates were returned to the xCELLigence RTCA MP instrument for an additional 24 hours of impedance/cell index measurements.

To calculate the percentage of specific tumor cell lysis, the cell index of tumor cells from coculture conditions were normalized to the cell index of tumor cells from tumor-only control wells using the equation shown below. The cell index is the measurement of focal cell adhesion within a well and is derived from the impedance value of a given well.

$$\% Killing = \frac{Cell Index ( tumor only ) - Cell Index ( experiment )}{Cell Index ( tumor only )} \times 100$$

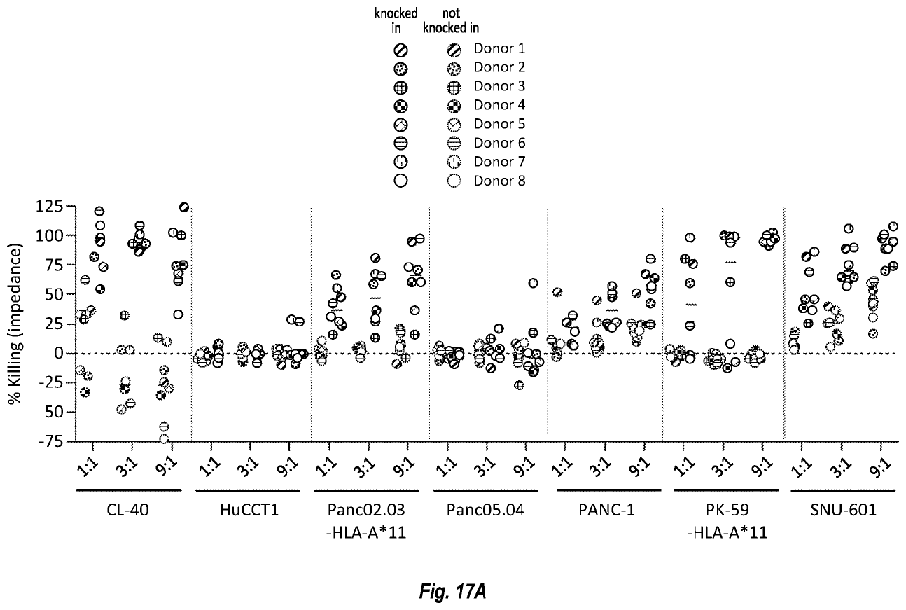

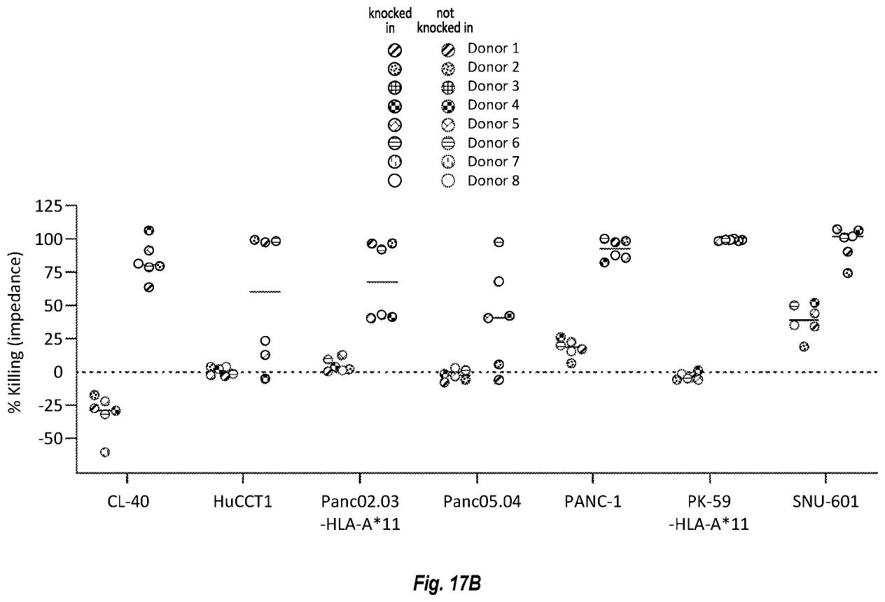

Percentage killing of HLA-A*11:01+ KRAS G12D-mutant tumor cell lines by KRAS G12D EE209_2 TCR knock in (KI) T cells at varying E:T ratios is shown in FIG. 17(A). Percentage killing of HLA-A*11:01⁺ KRAS G12D-mutant tumor cell lines pulsed with 10 mM 10-mer KRAS G12D peptide by KRAS G12D TCR KI T cells (E:T ratio=9:1) after tumor lines were pulsed with KRAS G12D 10-mer peptide is shown in FIG. 17(B). Circles represent individual donors. Filled circles represent TCR KI T cells and open circles represent TCR KO T cells. Matched border and fill colors indicate TCR KO and KI cells generated from the same donor.

### B. Coculture of T Cells and Tumor Cells for T Cell Activation Assay

Adherent tumor cell lines were plated at 1×10⁴ cells per well in 384-well Black/Clear Round Bottom Ultra-Low Attachment Spheroid Microplates (Corning; Glendale, AZ; Catalog No. 3830) for analysis of T cell activation (by flow cytometry and Luminex). After seeding, tumor cells were spun down for 1 minute at 1200 rpm to facilitate the formation of spheroids. Plated tumor cells were incubated at 37° C. overnight, and T cells were added to the cultures the following day. Cocultures were incubated in a humidified incubator at 37° C. with 5% CO₂ for 18 hours.

### C. Flow Cytometry to Measure T Cell Activation

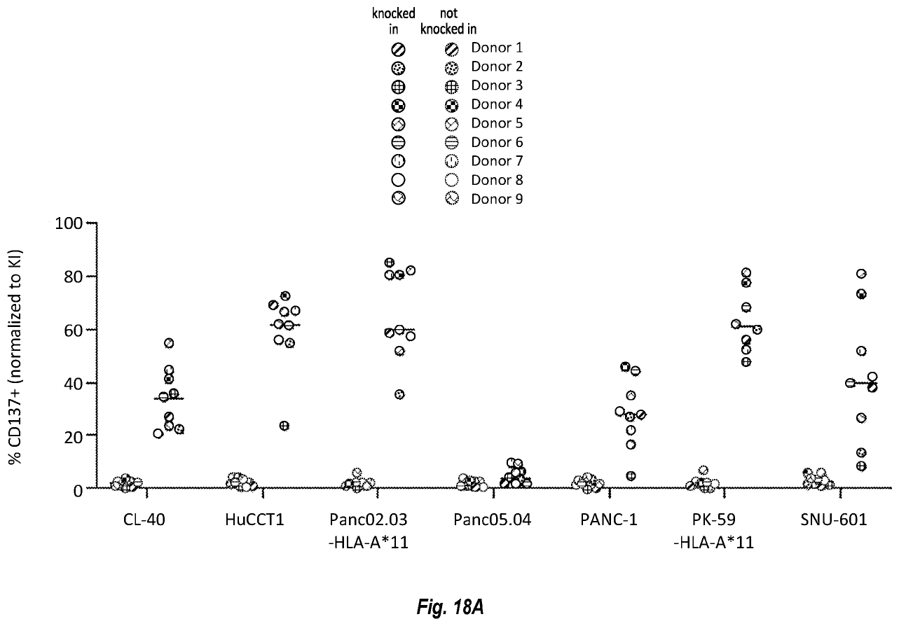

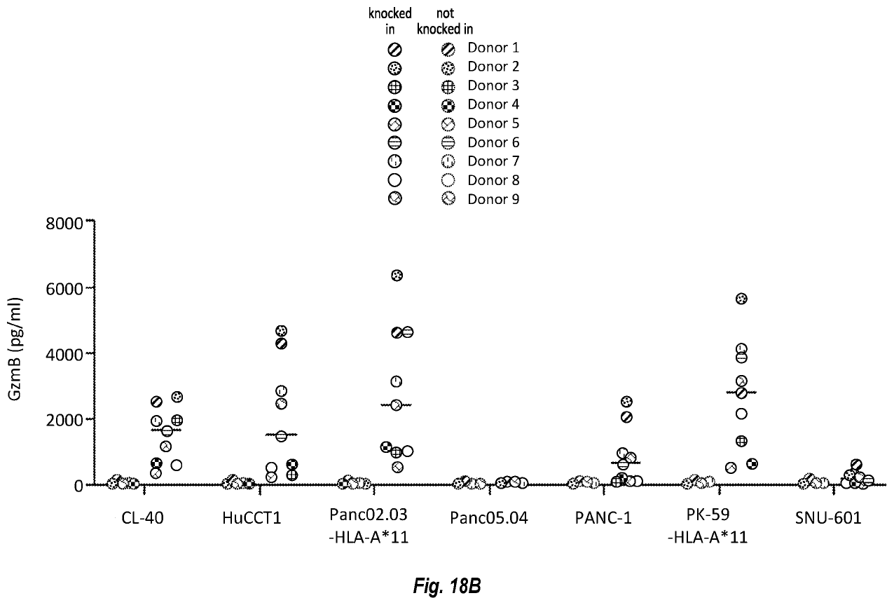

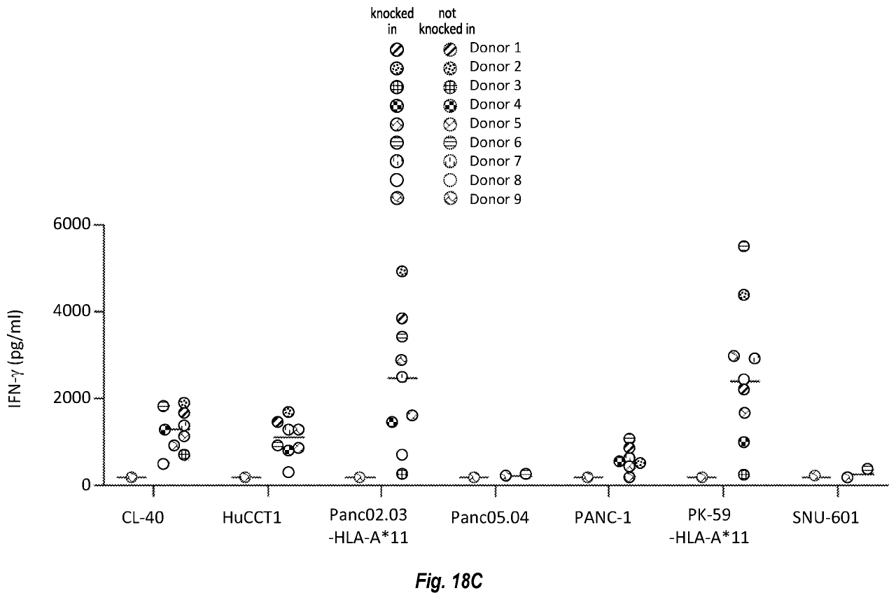

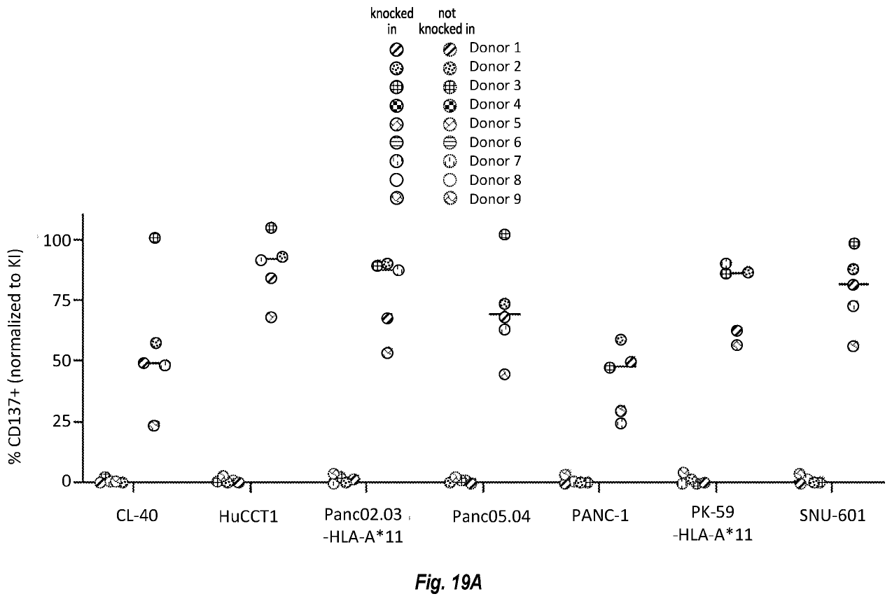

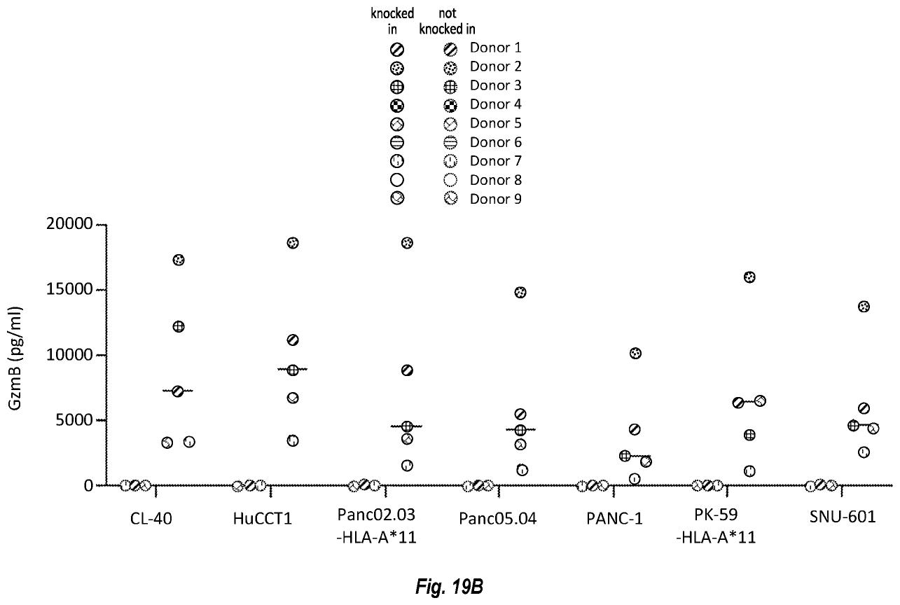

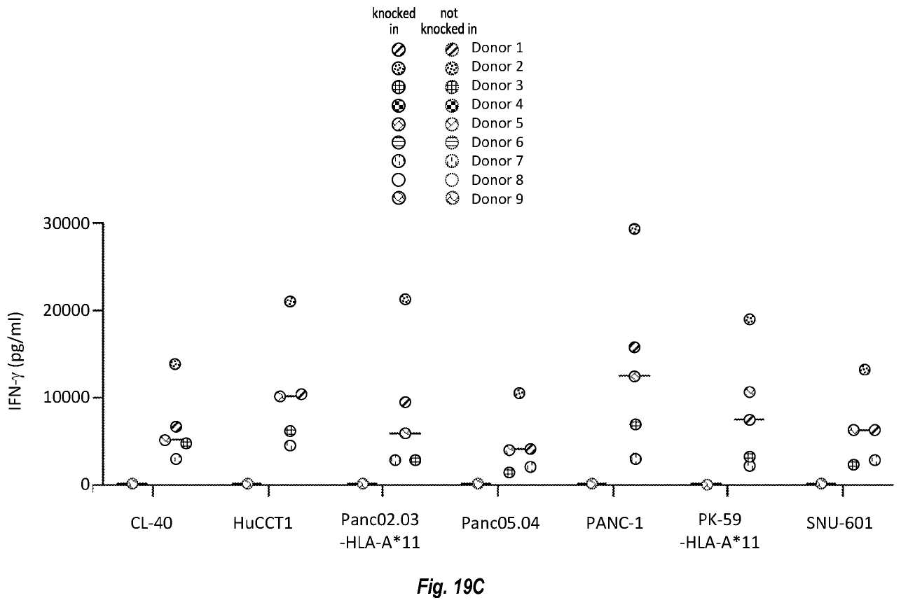

Cells were collected and consolidated into a 96-well V-bottom plate (Greiner Bio-One, Monroe, NC; Catalog No. 651201) using the Tecan Fluent 480 liquid handling instrument (Mannedorf, Switzerland). Cells were washed twice with 100 μL PBS at 1200 rpm for 5 minutes at room temperature, and then 100 μL of 1:1000 diluted Fixable Viability Dye eFluor™ 780 (see Table 4) was added to each well. The cells were incubated for 30 minutes on ice while protected from light. After 2 washes with FACS buffer (2% BSA in PBS), 25 μL of Fc block (Human TruStain FcX™) in FACS buffer was added and incubated on ice for 5 minutes. Then an equal volume of diluted antibody staining cocktail (CD8 BUV395; see Table 4) and CD137 Brilliant Violet 605™ (see Table 4) were added, and the cells were incubated on ice for 30 minutes while protected from light. Cells were washed twice with FACS buffer and then fixed with 1% paraformaldehyde. Cells were analyzed on a CytoFLEX LX Flow Cytometer (Beckman Coulter) and data were analyzed with FlowJo software. CD137+ T cells were identified and gated from CD8+ T cells from single live lymphocytes. The percentage of CD137 upregulation by KRAS G12D TCR KIT cells is shown in FIG. 18(A) and FIG. 19(A).

TABLE 4
Tumor Cell/T Cell Coculture Antibody Staining Information

| Antibody | Fluorophore | Clone | Company | Catalog No. | Concentration |
|---|---|---|---|---|---|
| Fixable | APCCy7 | NA | eBioscience | 65-0865-14 | 1:1000 |
| Viability | | | (San Diego, CA) | | |
| Dye | | | | | |
| CD8 | BUV395 | RPA-T8 | BD Biosciences. | 563795 | 1:40 |
| | | | (Franklin Lakes, NJ) | | |
| CD137 | BV-605 | 4B4-1 | BioLegend | 309822 | 1:40 |
| | | | (San Diego, CA) | | |

NA = not applicable.

### D. Luminex for Cytokine Quantification

Supernatants were analyzed for granzyme B and IFNγ using Luminex magnetic beads from the Human Luminex Discovery Assay kit (R&D Systems; Minneapolis, MN; Catalog No. LXSAHM-06) and a Luminex FLEXMAP 3D® reader (Austin, TX). Standards were prepared according to the manufacturer's protocol and diluted supernatants (1:2 dilution) were added to wells containing 6 μL of Luminex magnetic beads and incubated at 4° C. overnight with shaking (600 rpm). The next day, plates were warmed to room temperature, spun down for 2 minutes at 1200 rpm, and washed twice using a Blue® Washer magnetic carrier (BlueCatBio; Concord, MA). Six microliters of a 1:10 dilution of biotin antibody was added to each well and the plates were incubated using the following protocol: 1 minute of shaking using a Genie® microplate mixer set at 2.5, followed by 1 hour of shaking using an ORBI-SHAKER™ plate shaker (Benchmark Scientific; Sayreville, NJ) set at 600 rpm. After incubation, plates were washed twice using a Blu® Washer magnetic carrier and then incubated for an additional 30 minutes after the addition of 6 μL streptavidin-PE. Beads were washed twice, 40 μL of 1× wash buffer was added, and the samples were read using a Luminex FLEXMAP 3D® reader. Median fluorescence intensity values were exported to Genedata software (Basel, Switzerland) for 50% effective concentration (EC50) analysis and visualized using TIBCO Spotfire® (Palo Alto, CA). EC₅₀ values were calculated using a 4-parameter sigmoidal fit. The lower limit of detection (LLOD) for granzyme B and IFNγ was 42.7 and 206.2 μg/mL, respectively. Values below the LLOD were described as not detected. Granzyme B levels and IFNγ levels in culture supernatants are shown in FIG. 18(B)-(C) and FIG. 19(B)-(C).

### E. Conclusions

Human CD8+ T cells engineered to express the KRAS G12D-specific EE209_2 TCR exhibited killing activity against the majority of HLA A*11:01+ tumor cell lines with endogenous KRAS G12D expression (5 of 7 lines). The majority of HLA A*11:01+ KRAS G12D-mutant tumor cell lines also induced CD137 upregulation (7 of 7 lines), granzyme B production (6 of 7 lines), and IFNg production (5 of 7 lines) by KRAS G12D-specific CD8+ T cells. KRAS G12D-specific CD8+ T cells exhibited activity (killing, CD137 upregulation, and granzyme B and IFNg production) against all tumor cell lines when exogenous 10 mer KRAS G12D peptide was provided, indicating that all of the cell lines expressed sufficient HLA-A*11:01 molecules to activate T cells.

### Example 6. KRAS G12D Specific TCR as Monotherapy and in Combination with Atezolizumab

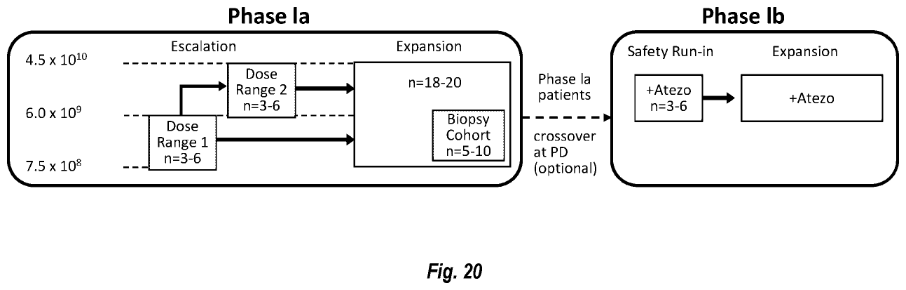

This example provides details of a Phase I, non-randomized open-label, multicenter, dose-escalation and expansion study designed to evaluate the safety, cellular kinetics, pharmacokinetics and preliminary anti-tumor activity of T cells engineered to express EE209_2 TCR (SEQ ID NOs: 12 and 13) (TCR T cell therapy) as a single agent (Phase Ia dose escalation and expansion stages) and in combination with atezolizumab (Phase Ib safety run-in and expansion stages). FIG. 20. This study will recruit patients with advanced pancreatic ductal adenocarcinoma (PDAC), colorectal cancer (CRC), or non-small cell lung cancer NSCLC who are HLA-A*11:01-positive and have KRAS G12D-positive tumors. Up to approximately 32 patients will be enrolled at approximately 15 sites globally.

In Phase 1a of the study, treatment with engineered T cells as a single agent will be evaluated in two stages, a dose-escalation stage and an expansion stage. Patients will undergo long-term follow-up (LFTU) assessments and monitoring for adverse events for up to 5 years after the engineered T cell infusion. Following disease progression, patients will have the opportunity to enroll in an optional Phase Ib crossover to treatment with atezolizumab. After the LTFU period, patients will be transitioned to a separate protocol for an extended follow-up period for up to 15 years after the engineered T cell infusion for continued monitoring of delayed adverse events.

### A. Primary Study Objectives

Primary Objective: To evaluate the safety of engineered T cells as a single agent and in combination with atezolizumab.

### Corresponding Endpoints:

Incidence and nature of dose limiting toxicities (DLTs)
Incidence and severity of adverse events, with severity determined according to National Cancer Institute's Common Terminology Criteria for Adverse Events (CTCAE) v5.0, except in cases of cytokine release syndrome (CRS) and immune effector cell-associated neurotoxicity syndrome (ICANS), which will be assessed according to (American Society of Transplantation and Cellular Therapy) ASTCT criteria
Incidence and severity of serious adverse events, adverse events of special interest, and select reportable new clinical conditions for up to five years after administration of engineered T cells
Change from baseline in selected vital signs
Change from baseline in selected clinical laboratory test results
Change from baseline in electrocardiograms (ECGs) and selected ECG parameters

### B. Secondary Study Objectives

Objective 1: To characterize the cellular kinetic profile of engineered T cells.

### Corresponding Endpoints:

Number of cells expressing EE209_2 TCR in blood at specified timepoints
Number of EE209_2 transgene copies in blood at specified timepoints

Objective 2: To make a preliminary assessment of the anti-tumor activity of engineered T cells as a single agent and in combination with atezolizumab.

### Corresponding Endpoints:

Objective response rate (ORR), defined as the proportion of patients with a complete response (CR) or partial response (PR) on two consecutive occasions ≥4 weeks apart, as determined by the investigator according to Response Evaluation Criteria in Solid Tumors (RECIST) v1.1

Objective 3: To evaluate the immune response to engineered T cells as a single agent and in combination with atezolizumab

### Corresponding Endpoints:

ORR, defined as the proportion of patients with a CR or PR on two consecutive occasions ≥4 weeks apart, as determined by the investigator according to RECIST v1.1
Prevalence of anti-drug antibodies (ADAs) to EE209_2 TCR at baseline and incidence of ADAs to EE209_2 TCR during the study

### C. Exploratory Study Objectives

To evaluate potential relationships between engineered T cell dose/selected covariates and the exposure of engineered T celk.

To evaluate potential relationships between dose/exposure of engineered T cell and response.

To characterize the pharmacokinetics of atezolizumab when given in combination with engineered T cell (Phase Ib only).

To make a preliminary assessment of the anti-tumor activity of engineered T cells as a single agent and in combination with atezolizumab.

To evaluate the immune response to atezolizumab when given in combination with engineered T cells.

To evaluate potential effects of EE209_2 TCR and atezolizumab ADAs.

To evaluate cellular immunogenicity to engineered T cells and potential effects of cellular immunogenicity.

Preliminary assessment of biomarkers that are predictive of response to engineered T cells when administered as a single agent (Phase Ia) or in combination with the anti-PD-L1 antibody, atezolizumab, (Phase Ib) (i.e., predictive biomarkers), can provide evidence of engineered T cells activity (i.e., pharmacodynamic biomarkers), are associated with progression to a more severe disease state (i.e., prognostic biomarkers).

Preliminary assessment of biomarker that are associated with resistance to engineered T cells, are associated with susceptibility to developing adverse events or can lead to improved adverse event monitoring or investigation (i.e., safety biomarkers), or can increase the knowledge and understanding of disease biology and drug safety or pharmacokinetics.

### D. Screening

### 1. Screening Part 1

Patients who sign consent for the study will undergo initial determination of eligibility based on tumor type, HLA-A*11:01 expression, presence of KRAS G12D mutation, and infectious disease testing. HLA-A*11:01 and KRAS G12D mutation testing will be performed centrally. Patients may receive other anti-cancer therapies while participating in Screening Part 1. Patients who meet eligibility criteria during Screening Part 1 may continue with Screening Part 2.

### 2. Screening Part 2 and Leukapheresis

Screening Part 2 is a determination of eligibility for enrollment based on review of all inclusion and exclusion criteria, provided in part herein and typical of clinical trials of this nature. Patients who meet eligibility criteria during Screening Part 2 will undergo leukapheresis to collect T cells for manufacturing of engineered T cells. Manufacturing of engineered T cells may take approximately 5 to 6 weeks. Patients may receive other anti-cancer therapies (hereafter referred to as bridging therapy) while participating in Screening Part 2 and waiting for the completion of engineered T cell manufacturing. However, patients will need to follow wash-out periods for anti-cancer therapy.

### 3. Screening Part 3

Approximately 14 days prior to the start of the lymphodepleting chemotherapy regimen, eligibility will be re-confirmed by review of all inclusion and exclusion criteria. Patients who meet eligibility criteria during Screening Part 3 may start the lymphodepleting chemotherapy regimen.

### E. Lymphodepleting Chemotherapy

Receipt of engineered T cells at the clinical site should be confirmed prior to starting lymphodepleting chemotherapy. A lymphodepleting chemotherapy regimen will occur prior to administration of the engineered T cells.

### F. Phase Ia Treatment

Patients in the Phase Ia portion of the study will receive engineered T cells as a single dose IV infusion on Day 1 of the study. Phase Ia will be enrolled in two stages: a dose-escalation stage and an expansion stage, as shown in FIG. 20.

### 1. Phase Ia Dose-Escalation Stage

Between 6 to 12 patients will be enrolled in the dose-escalation stage. At least two dose ranges, with a defined minimum and maximum permitted total dose of engineered T cells will be evaluated. In the first dose range (DR1), patients will receive at least 7.5×10⁸ total cells and may receive up to a maximum dose of 6×10⁹ total cells. In the second dose range (DR2), patients will receive greater than 6×10⁹ total cells up to a maximum dose of 4.5×10¹⁰ total cells. Each dose range will enroll 3 to 6 patients in accordance with the dose-escalation rules described below. RO7658589 administration between the first and second patient in each dose range will be separated by a minimum of 3 weeks. RO7658589 administration between each subsequent patient must be separated by a minimum of 2 weeks. Engineered T cell will be administered as a single infusion on Day 1. Following RO7578589 infusion, all patients will be hospitalized for a minimum of 7 days and will remain hospitalized until pre-specified criteria are met for discharge or at the investigator's discretion.

Engineered T cells are made on a per-patient basis, and heterogeneity is expected in the number and attributes of engineered T cells manufactured for each patient. Administered doses will be based on what can be manufactured for administration and may fall anywhere within the range of doses allowed by the protocol. For any dose range, if the number of cells that are manufactured is above the maximum dose allowed for the assigned dose range, the manufactured dose will be administered up to the maximum limit for that dose range. If the number of engineered T cells manufactured is below the minimum dose required for the assigned dose range (but higher than the minimum dose acceptable in the lowest dose range (i.e., 7.5×10⁸ total cells), patients may be enrolled and treated in the expansion stage at a dose that falls within any lower dose range that has cleared DLT evaluation. Patients whose total manufactured product does not equal or exceed 7.5×10⁸ cells will be ineligible for treatment.

Patients will be closely monitored for adverse events during the DLT assessment window, defined as Day 1 through Day 28. Adverse events identified as DLTs, as defined below, will be reported to the Sponsor within 24 hours.

### 2. Dose-Escalation Rules

This study will evaluate different dose ranges of engineered T cells. If the MTD is exceeded at any dose range tested, additional patients may be enrolled to evaluate engineered T cells at a lower dose range with agreement of the Internal Safety Committee (ISC).

Dose escalation for engineered T cells will occur in accordance with the rules listed below:

A minimum of 3 patients will initially be enrolled in each dose range.

If none of the first 3 DLT-evaluable patients experiences a DLT and in agreement with the ISC, enrollment at the next higher dose range may proceed. In the Phase Ia portion, enrollment in the expansion stage at or below the current dose range will be enabled.

If 1 of the first 3 DLT-evaluable patients experiences a DLT, the dose range or a portion of that dose range will be expanded to 6 patients. If there are no further DLTs in the first 6 DLT-evaluable patients and in agreement with the ISC, enrollment at the next higher dose range may proceed. In the Phase Ia portion, enrollment in the Phase Ia expansion stage at or below the current dose range will be enabled.

If 2 or more DLT-evaluable patients in a dose range experience a DLT, the MTD will have been exceeded and dose escalation will stop. An additional 3 patients will be evaluated for DLTs at the preceding dose range, unless 6 patients have already been evaluated at that range. Additional patients may also be evaluated at intermediate dose ranges that may overlap with previously evaluated dose ranges, if agreed upon by the ISC.

If the MTD is exceeded at any dose range, the highest dose range at which fewer than 2 of 6 DLT-evaluable patients (i.e., <33%) experience a DLT will be evaluated by the ISC in order to determine the MTD.

If the MTD is not exceeded at any dose range, the highest dose range administered in this study will be evaluated by the ISC in order to determine the MAD. Additional patients may also be evaluated at higher dose ranges, if agreed upon by the ISC.

Based on a review of real-time safety data and available preliminary CK data, including adverse events that occur outside of the DLT assessment window, dose escalation may be halted or modified by the Sponsor as deemed appropriate.

### 3. Phase Ia Expansion Stage

Up to 20 patients will be enrolled in the expansion stage, of which approximately 5-10 patients will be enrolled in a mandatory biopsy cohort. Patients in the expansion stage will be treated at or below the highest dose range deemed tolerable and agreed upon by the ISC.

The expansion stage will open once the DR1 (from 7.5×10⁸ to 6×10⁹ total cells) dose escalation cohort has cleared its DLT assessment period. During accrual of the DR2 cohort (from >6×10⁹ to 4.5×10¹⁰ total cells), patients whose total manufactured product does not exceed 6×10⁹ total cells will be permitted to enroll in the expansion stage at DR1. Clearance of DR2 then enables the full dose range (7.5×10⁸ total cells−4.5×10¹⁰ total cells) enabled by the manufacturing process in the expansion stage. Enrollment in the expansion stage will always be at or below the highest dose range that has cleared in the dose-escalation stage. Once the MTD or MAD is determined, enrollment in the expansion stage must be at or below the MTD or MAD. Patients whose total manufactured product is below the minimum dose acceptable in the lowest dose range (i.e., 7.5×10⁸ total cells) will be ineligible for treatment but may be considered for a second trial of leukapheresis and engineered T cell manufacture.

Patients will be treated in the expansion stage to obtain additional safety, tolerability, biomarker, and cellular kinetics data, as well as preliminary evidence of clinical activity. If the frequency of Grade 3 or 4 toxicities or other unacceptable toxicities at any dose range in the expansion stage suggests that the MTD has been exceeded, accrual at that dose range will be halted. Continued enrollment at lower dose ranges in the expansion stage may continue, with the agreement of the ISC.

To explore potential tumor markers of PD activity of engineered T cell, approximately 5-10 patients within the Phase Ia expansion stage will be enrolled in a mandatory biopsy cohort. These patients must have safely accessible tumor lesions, as they will be required to undergo a biopsy approximately 28 to 56 days after the engineered T cell infusion. The biopsy cohort within the Phase Ia expansion stage is designed to obtain early evidence of biologic activity and to characterize pharmacodynamic modulation of engineered T cells. Additional, optional biopsies may also be collected at the investigator's discretion and with the consent of the patient, preferably at the time of radiographic response or progression.

### G. Phase 1b Treatment

The Phase Ib portion of the study will assess the safety of atezolizumab treatment in patients who had previously received engineered T cells in the Phase Ia portion of the study. The Phase Ib may be open only after DR1 in the Phase Ia portion of the study has cleared its DLT assessment period.

1. Phase Ib Eligibility and Treatment with Atezolizumab

Patients treated with engineered T cells as a single agent in the Phase Ia portion of the study who have radiographically documented disease progression may be eligible to receive treatment with atezolizumab in the Phase Ib portion after their engineered T cell infusion to minimize potential for atezolizumab to exacerbate the severity of engineered T cell-related toxicities during the cell-expansion phase. Eligibility criteria for Phase Ib, including the presence of detectable engineered T cells in peripheral blood or detectable EE209 2 TCR in peripheral blood or tumor tissue. Lymphodepleting chemotherapy and engineered T cell will not be re administered.

Assessments obtained at the last visit in the Phase Ia portion of the study may be used as crossover screening assessments for treatment with atezolizumab in the Phase Ib portion. The following crossover screening assessments must be repeated/obtained within 14 days prior to the first day of starting treatment with atezolizumab, in order to re-establish baseline pretreatment clinical and disease status: limited physical examination, ECOG status, hematology and serum chemistry laboratory tests, amylase, lipase, thyroid function tests, and urine analysis.

A radiographic tumor assessment, including a CT scan of the chest, abdomen, and pelvis, must also be performed, unless already performed to document disease progression, within 4 weeks prior to the first day of starting treatment with atezolizumab, as this assessment becomes the new baseline scan for the patient starting treatment in the Phase Ib portion of the study.

Treatment day numbering will not re-start when a patient starts atezolizumab treatment in the Phase Ib portion (i.e., all activities are relative to the day of engineered T cell infusion in Part Ia).

Eligible patients will receive a fixed dose of atezolizumab 1680 mg IV infusion every 4 weeks (Q4W) (i.e., 1680 mg on Day 1 of each 28-day cycle) until unacceptable toxicity or loss of clinical benefit as determined by the investigator after an integrated assessment of radiographic and biochemical data, local biopsy results (if available), and clinical status (e.g., symptomatic deterioration such as pain secondary to disease). Because of the possibility of an initial increase in tumor burden caused by immune-cell infiltration in the setting of a T cell response (termed “pseudoprogression”) with atezolizumab treatment, radiographic progression per RECIST v1.1 may not be indicative of true disease progression. In the absence of unacceptable toxicity, patients who meet criteria for disease progression per RECIST v1.1 while receiving atezolizumab will be permitted to continue atezolizumab if they meet all of the following criteria:
Evidence of clinical benefit, as determined by the investigator following a review of all available data
Absence of symptoms and signs (including laboratory values, such as new or worsening hypercalcemia) indicating unequivocal progression of disease
Absence of decline in ECOG Performance Status that can be attributed to disease progression
Absence of tumor progression at critical anatomical sites (e.g., leptomeningeal disease) that cannot be managed by protocol-allowed medical interventions

### 2. Phase Ib Safety Run-In and Expansion

The Phase Ib will include a safety run-in and an expansion stage, as shown in FIG. 20. The safety run-in will initially enroll approximately 3-6 patients. Enrollment of the first 3 patients will be staggered such that atezolizumab is administered ≥1 week between each patient. After the first 3 patients have completed 28 days after atezolizumab infusion, the ISC will review all safety data to determine whether study treatment is tolerable, in which there is an acceptable assessment of risk by the ISC in consultation with the investigators to allow additional patients to receive atezolizumab. If study treatment is tolerable, the Sponsor will then enroll approximately 3 additional patients in the safety run-in to further assess safety and tolerability of atezolizumab. If study treatment is deemed tolerable in the safety run-in by the ISC and in consultation with investigators after a minimum of 6 patients have enrolled and have completed 28 days of study treatment, enrollment may begin in the expansion stage.

### H. Duration of Participation

Treatment will continue until disease progression per RECIST v1.1. The total duration of study participation for each patient is expected to range from 1 day to up to 5 years. Starting at Year 6, patients will be transitioned to a separate extended LTFU protocol for up to 15 years after engineered T cell infusion for continued monitoring of delayed adverse events.

### I. Inclusion Criteria

Patients must meet at least the following inclusion criteria, provided in part herein, for study entry:

### 1. Inclusion Criteria for Screening Part 1

Patients must meet the following criteria for inclusion in Screening Part 1:
Histologic or cytologic documentation of one of the following unresectable, locally advanced or metastatic cancer types: pancreatic ductal adenocarcinoma, colorectal cancer, and non-small cell lung cancer

### 2. Inclusion Criteria for Screening Part 2 and Part 3

Patients must meet the following additional inclusion criteria for study entry:
HLA-A*11:01-positive
KRAS G12D mutation-positive determined by central testing or local testing using a sponsor-approved assay and after review and acceptance of local results by the sponsor
Measurable disease per RECIST v1.1

### 3. Additional Inclusion Criteria for Screening Part 3

Patients must meet the following additional inclusion criteria for study entry at screening Part 3:
Manufactured engineered T cell product for patient meets minimum total cell dose requirement (at least 7.5×10⁸ total cells)
Disease that has progressed after at least one available standard therapy; or for whom standard therapy has proven to be ineffective or intolerable or is considered inappropriate; or for whom a clinical trial of an investigational agent is a recognized standard of care
For patients with PDAC, the following criteria must be met:
   Histologically or cytologically confirmed metastatic PDAC
   Disease progression with or intolerance to either 5FU- or gemcitabine-based first-line chemotherapy.
   Patients with endocrine or acinar pancreatic carcinoma are not eligible for the study.
For patients with CRC, the following criteria must be met:
   Histologically confirmed metastatic adenocarcinoma of the colon or rectum
   Mismatch repair (MMR)/microsatellite instability (MSI) status must be known
   Patients with dMMR/MSI-H should have received prior treatment with an approved immunotherapy (e.g., anti-PD-1/anti-PD-L1 with or without anti-CTLA4 therapy), if available and approved by local regulatory authorities.
   Disease progression with or intolerance to a fluoropyrimidine-based regimen that includes either oxaliplatin (e.g., FOLFOX, CAPEOX) or irinotecan (e.g., FOLFIRI)
   Patients with tumors of appendiceal origin are not eligible for the study.
For patients with NSCLC, the following criteria must be met:
   Histologically confirmed unresectable, locally advanced or metastatic adenocarcinoma of the lung
   Disease progression with or intolerance to single-agent or combination therapy with an investigational or approved PD-L1/PD-1 inhibitor.
   Patients whose tumors have a targetable somatic alteration, including those involving EGFR, ALK, ROS1, BRAFV600E, NTRK, MET, RET and KRAS G12C must have experienced disease progression with or intolerance to treatment with a targeted agent, if available and approved by local regulatory authorities

### 4. Additional Inclusion Criteria for Phase Ib Portion of the Study

Patients crossing over to the Phase Ib portion of the study must meet either one of the following criteria:
Patients transferring into the Phase Ib portion of the study must also meet all of the following criteria:
Presence of detectable engineered T cells in peripheral blood or EE209 2 TCR is detectable in the peripheral blood or in tumor as determined by central testing
Resolution to either Grade 1 or better, or to baseline, of any engineered T cell-related adverse events in the Phase Ia portion of the study on or before initiation of treatment with atezolizumab
RECIST target lesions should not be biopsied
Treatment with atezolizumab after disease progression following engineered T cell infusion must be considered acceptable as determined after a careful assessment of available treatment options and discussion of the benefit-risk balance with the patient by the investigator and in consultation with the Medical Monitor.

### J. Study Treatment Formulation and Packaging

### 1. Engineered T Cells

Engineered T cells will be supplied by the Sponsor as a cell suspension at 7.5×10⁷ cells/mL concentration.

### 2. Atezolizumab

Atezolizumab will be supplied by the Sponsor as a sterile liquid in a single-use, 20-mL glass vial. The vial contains approximately 20 mL (1200 mg) of atezolizumab solution.

### 3. Lymphodepleting Chemotherapy

Lymphodepleting chemotherapy regimen consisting of cyclophosphamide and fludarabine will be supplied by the site.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, all numbers in the specification may be considered to be modified by the term about. The term about refers to a numeric value, including, for example, whole numbers, fractions, and percentages, whether or not explicitly indicated. The fact that ranges are provided for some numbers and not others does not alter that each is modified by the term about. The term about generally refers to a range of numerical values (e.g., +/−10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

### APPENDIX A: SEQUENCE SUMMARY

### DESCRIPTION OF THE SEQUENCES

### APPENDIX A PROVIDES A LISTING OF CERTAIN SEQUENCES REFERENCED HEREIN. THE AMINO ACID SEQUENCES PROVIDED ARE FROM N-TERMINUS TO C-TERMINUS. THE NUCLEIC ACID SEQUENCES ARE FROM 5′ TO 3′.

| SEQ ID | | |
|---|---|---|
| NO. | NAME | SEQUENCE |
| 1 | 10-MER KRAS | VVVGADGVGK |
| | G12D-DERIVED | |
| | PEPTIDE | |
| 2 | 9-MER KRAS | VVGADGVGK |
| | G12D-DERIVED | |
| | PEPTIDE | |
| 3 | EE209_KRAS_2/ | WSHSY |
| | EE209_2 TCR | |
| | BETA CDR1B | |
| 4 | EE209_2 TCR | SAAADI |
| | BETA CDR2B | |
| 5 | UNASSIGNED | N/A |
| 6 | EE209_KRAS_2/ | CASSVGRGGTEAFF |
| | EE209_2 TCR | |
| | BETA CDR3B | |
| 7 | EE209_KRAS 2/ | SSNFYA |
| | EE209_2 TCR | |
| | alpha CDRla | |
| 8 | EE209_KRAS 2/ | MTLNGDE |
| | EE209_2 TCR | |
| | alpha CDR2a | |
| 9 | EE209_KRAS 2/ | CAFLPGYSTLTF |
| | EE209_2 TCR | |
| | alpha CDR3a | |
| 10 | EE209_KRAS 2/ | ILNVEQSPQSLHVQEGDSTNFTCSFPSSNFYALHWYRWETAKSP |
| | EE209_2 TCR | EALFVMTLNGDEKKKGRISATLNTKEGYSYLYIKGSQPEDSATY |
| | alpha variable | LCAF |
| | region | |
| 11 | EE209_KRAS 2/ | DAGITQSPRYKITETGRQVTLMCHQTWSHSYMFWYRQDLGHGLR |
| | EE209_2 TCR | LIYYSAAADITDKGEVPDGYVVSRSKTENFPLTLESATRSQTSV |
| | beta variable | YFCASSV |
| | region | |
| 12 | EE209_KRAS_2/ | MEKNPLAAPLLILWFHLDCVSSILNVEQSPQSLHVQEGDSTNFT |
| | EE209_2 TCR | CSFPSSNFYALHWYRWETAKSPEALFVMTLNGDEKKKGRISATL |
| | alpha | NTKEGYSYLYIKGSQPEDSATYLCAFLPGYSTLTFGKGTMLLVS |
| | | PDIQNPDPAVYQLRDSKSSDKSVCLFTDFDSQTNVSQSKDSDVY |
| | | ITDKTVLDMRSMDFKSNSAVAWSNKSDFACANAFNNSIIPEDTF |
| | | FPSPESSCDVKLVEKSFETDTNLNFQNLSVIGFRILLLKVAGFN |
| | | LLMTLRLWSS |
| 13 | EE209_KRAS 2/ | MGTRLFFYVALCLLWAGHRDAGITQSPRYKITETGRQVTLMCHQ |
| | EE209_2 TCR | TWSHSYMFWYRQDLGHGLRLIYYSAAADITDKGEVPDGYVVSRS |
| | beta using | KTENFPLTLESATRSQTSVYFCASSVGRGGTEAFFGQGTRLTVV |
| | TRBC2-201 | EDLKNVFPPKVAVFEPSEAEISHTQKATLVCLATGFYPDHVELS |
| | (Bold and | WWVNGKEVHSGVSTDPQPLKEQPALNDSRYCLSSRLRVSATFWQ |
| | underlined | NPRNHFRCQVQFYGLSENDEWTQDRAKPVTQIVSAEAWGRADCG |
| | portions are | FTSESYQQGVLSATILYEILLGKATLYAVLVSALVLMAMVKRKD |
| | differences with | SRG |
| | SEQ ID NO: 14) | |
| 14 | EE209_KRAS 2/ | MGTRLFFYVALCLLWAGHRDAGITQSPRYKITETGRQVTLMCHQ |
| | EE209_2 TCR | TWSHSYMFWYRQDLGHGLRLIYYSAAADITDKGEVPDGYVVSRS |
| | beta using | KTENFPLTLESATRSQTSVYFCASSVGRGGTEAFFGQGTRLTVV |
| | TRBC1-201 | EDLNKVFPPEVAVFEPSEAEISHTQKATLVCLATGFFPDHVELS |
| | (Bold and | WWVNGKEVHSGVSTDPQPLKEQPALNDSRYCLSSRLRVSATFWQ |
| | underlined | NPRNHFRCQVQFYGLSENDEWTQDRAKPVTQIVSAEAWGRADCG |
| | portions are | FTSVSYQQGVLSATILYEILLGKATLYAVLVSALVLMAMVKRKD |
| | differences with | F |
| | SEQ ID NOs: 13) | |
| 15 | EE209_KRAS 2/ | MGTRLFFYVALCLLWAGHRDAGITQSPRYKITETGRQVTLMCHQ |
| | EE209_2 TCR | TWSHSYMFWYRQDLGHGLRLIYYSAAADITDKGEVPDGYVVSRS |
| | beta using | KTENFPLTLESATRSQTSVYFCASSVGRGGTEAFFGQGTRLTVV |
| | TRBC2-202 | EDLKNVFPPEVAVFEPSEAEISHTQKATLVCLATGFYPDHVELS |
| | (Bold and | WWVNGKEVHSGVSTDPQPLKEQPALNDSRYCLSSRLRVSATFWQ |
| | underlined | NPRNHFRCQVQFYGLSENDEWTQDRAKPVTQIVSAEAWGRADCG |
| | portions are | FTSESYQQGVLSATILYEILLGKATLYAVLVSALVLMAMVKRKD |
| | differences with | SRG |
| | SEQ ID NO: 13) | |
| 16 | EE184_KRAS_8A | LNHDA |
| | TCR beta CDR1b | |
| 17 | EE184_KRAS_8A | SQIVND |
| | TCR beta CDR2b | |
| 18 | EE184_KRAS_8A | CASSTGAGAFRTEAFF |
| | TCR beta CDR3b | |
| 19 | EE184_KRAS_8A | TSESDYY |
| | TCR alpha CDRla | |
| 20 | EE184_KRAS_8A | QEAYKQQN |
| | TCR alpha CDR2a | |
| 21 | EE184_KRAS_8A | CAYKKYNDMRF |
| | TCR alpha CDR3a | |
| 22 | EE184_KRAS_8A | AQTVTQSQPEMSVQEAETVTLSCTYDTSESDYYLFWYKQPPSRQ |
| | TCR alpha | MILVIRQEAYKQQNATENRFSVNFQKAAKSFSLKISDSQLGDAA |
| | variable region | MYFCAYK |
| 23 | EE184_KRAS_8A | DGGITQSPKYLFRKEGQNVTLSCEQNLNHDAMYWYRQDPGQGLR |
| | TCR beta variable | LIYYSQIVNDFQKGDIAEGYSVSREKKESFPLTVTSAQKNPTAF |
| | region | YLCASST |
| 24 | EE184_KRAS_8A | MACPGFLWALVISTCLEFSMAQTVTQSQPEMSVQEAETVTLSCT |
| | TCR alpha chain | YDTSESDYYLFWYKQPPSRQMILVIRQEAYKQQNATENRFSVNF |
| | | QKAAKSFSLKISDSQLGDAAMYFCAYKKYNDMRFGAGTRLTVKP |
| | | NIQNPDPAVYQLRDSKSSDKSVCLFTDFDSQTNVSQSKDSDVYI |
| | | TDKTVLDMRSMDFKSNSAVAWSNKSDFACANAENNSIIPEDTFF |
| | | PSPESSCDVKLVEKSFETDTNLNFQNLSVIGFRILLLKVAGFNL |
| | | LMTLRLWSS |
| 25 | EE184_KRAS_8A | MSNQVLCCVVLCFLGANTVDGGITQSPKYLFRKEGQNVTLSCEQ |
| | TCR beta chain | NLNHDAMYWYRQDPGQGLRLIYYSQIVNDFQKGDIAEGYSVSRE |
| | | KKESFPLTVTSAQKNPTAFYLCASSTGAGAFRTEAFFGQGTRLT |
| | | VVEDLNKVFPPEVAVFEPSEAEISHTQKATLVCLATGFFPDHVE |
| | | LSWWVNGKEVHSGVSTDPQPLKEQPALNDSRYCLSSRLRVSATF |
| | | WQNPRNHFRCQVQFYGLSENDEWTQDRAKPVTQIVSAEAWGRAD |
| | | CGFTSVSYQQGVLSATILYEILLGKATLYAVLVSALVLMAMVKR |
| | | KDF |
| 26 | PD6_KRASMUT_ | SQVTM |
| | 22 TCR beta | |
| | CDR1b | |
| 27 | PD6_KRASMUT_ | ANQGSEA |
| | 22 TCR beta | |
| | CDR2b | |
| 28 | PD6_KRASMUT_ | CSVTWAGGWSYGYTF |
| | 22 TCR beta | |
| | CDR3b | |
| 29 | PD6_KRASMUT_ | TSESDYY |
| | 22 TCR alpha | |
| | CDR1a | |
| 30 | PD6_KRASMUT_ | QEAYKQQN |
| | 22 TCR alpha | |
| | CDR2a | |
| 31 | PD6_KRASMUT_ | CAYYSGGGADGLTF |
| | 22 TCR alpha | |
| | CDR3a | |
| 32 | PD6_KRASMUT_ | AQTVTQSQPEMSVQEAETVTLSCTYDTSESDYYLFWYKQPPSRQ |
| | 22 TCR alpha | MILVIRQEAYKQQNATENRFSVNFQKAAKSFSLKISDSQLGDAA |
| | variable region | MYFCAYYS |
| 33 | PD6_KRASMUT_ | SAVISQKPSRDICQRGTSLTIQCQVDSQVTMMFWYRQQPGQSLT |
| | 22 TCR beta_ | LIATANQGSEATYESGFVIDKFPISRPNLTFSTLTVSNMSPEDS |
| | variable region | SIYLCSVT |
| 34 | PD6_KRASMUT_ | MACPGFLWALVISTCLEFSMAQTVTQSQPEMSVQEAETVTLSCT |
| | 22 TCR alpha | YDTSESDYYLFWYKQPPSRQMILVIRQEAYKQQNATENRFSVNF |
| | chain | QKAAKSFSLKISDSQLGDAAMYFCAYYSGGGADGLTFGKGTHLI |
| | | IQPYIQNPDPAVYQLRDSKSSDKSVCLFTDFDSQTNVSQSKDSD |
| | | VYITDKTVLDMRSMDFKSNSAVAWSNKSDFACANAFNNSIIPED |
| | | TFFPSPESSCDVKLVEKSFETDTNLNFQNLSVIGFRILLLKVAG |
| | | FNLLMTLRLWSS |
| 35 | PD6_KRASMUT_ | MLSLLLLLLGLGSVFSAVISQKPSRDICQRGTSLTIQCQVDSQV |
| | 22 TCR beta | TMMFWYRQQPGQSLTLIATANQGSEATYESGFVIDKFPISRPNL |
| | chain | TFSTLTVSNMSPEDSSIYLCSVTWAGGWSYGYTFGSGTRLTVVE |
| | | DLNKVFPPEVAVFEPSEAEISHTQKATLVCLATGFFPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSVSYQQGVLSATILYEILLGKATLYAVLVSALVLMAMVKRKDF |
| 36 | PD6_KRASMUT_ | SGHNS |
| | 9 TCR beta | |
| | CDR1b | |
| 37 | PD6_KRASMUT_ | FNNNVP |
| | 9 TCR beta | |
| | CDR2b | |
| 38 | PD6_KRASMUT_ | CASSPGPGRHNSPLHF |
| | 9 TCR beta | |
| | CDR3b | |
| 39 | PD6_KRASMUT_ | TRDTTYY |
| | 9 TCR alpha | |
| | CDR1a | |
| 40 | PD6_KRASMUT_ | RNSFDEQN |
| | 9 TCR alpha | |
| | CDR2a | |
| 41 | PD6_KRASMUT_ | CALSELTTSGTYKYIF |
| | 9 TCR alpha | |
| | CDR3a | |
| 42 | PD6_KRASMUT_ | AQKVTQAQTEISVVEKEDVTLDCVYETRDTTYYLFWYKQPPSGE |
| | 9 TCR alpha | LVFLIRRNSFDEQNEISGRYSWNFQKSTSSFNFTITASQVVDSA |
| | variable region | VYFCALSE |
| 43 | PD6_KRASMUT_ | DAGVIQSPRHEVTEMGQEVTLRCKPISGHNSLFWYRQTMMRGLE |
| | 9 TCR beta | LLIYFNNNVPIDDSGMPEDRFSAKMPNASFSTLKIQPSEPRDSA |
| | variable region | VYFCASSP |
| 44 | PD6_KRASMUT_ | MLTASLLRAVIASICVVSSMAQKVTQAQTEISVVEKEDVTLDCV |
| | 9 TCR alpha | YETRDTTYYLFWYKQPPSGELVFLIRRNSFDEQNEISGRYSWNF |
| | chain | QKSTSSFNFTITASQVVDSAVYFCALSELTTSGTYKYIFGTGTR |
| | | LKVLANIQNPDPAVYQLRDSKSSDKSVCLFTDFDSQTNVSQSKD |
| | | SDVYITDKTVLDMRSMDFKSNSAVAWSNKSDFACANAFNNSIIP |
| | | EDTFFPSPESSCDVKLVEKSFETDTNLNFQNLSVIGFRILLLKV |
| | | AGFNLLMTLRLWSS |
| 45 | PD6_KRASMUT_ | MDSWTFCCVSLCILVAKHTDAGVIQSPRHEVTEMGQEVTLRCKP |
| | 9 TCR beta chain | ISGHNSLFWYRQTMMRGLELLIYFNNNVPIDDSGMPEDRFSAKM |
| | | PNASFSTLKIQPSEPRDSAVYFCASSPGPGRHNSPLHFGNGTRL |
| | | TVTEDLNKVFPPEVAVFEPSEAEISHTQKATLVCLATGFFPDHV |
| | | ELSWWVNGKEVHSGVSTDPQPLKEQPALNDSRYCLSSRLRVSAT |
| | | FWQNPRNHFRCQVQFYGLSENDEWTQDRAKPVTQIVSAEAWGRA |
| | | DCGFTSVSYQQGVLSATILYEILLGKATLYAVLVSALVLMAMVK |
| | | RKDF |
| 46 | PD22_KRASMUT | MDHEN |
| | WT_1 TCR beta | |
| | CDR1b | |
| 47 | PD22_KRASMUT | SYDVKM |
| | WT_1 TCR beta | |
| | CDR2b | |
| 48 | PD22_KRASMUT | CASSLGPGQHNSPLHF |
| | WT_1 TCR beta | |
| | CDR3b | |
| 49 | PD22_KRASMUT | TRDTTYY |
| | WT_1 TCR | |
| | alpha CDR1a | |
| 50 | PD22_KRASMUT | RNSFDEQN |
| | WT_1 TCR | |
| | alpha CDR2a | |
| 51 | PD22_KRASMUT | CALSEALAGGSYIPTF |
| | WT_1 TCR | |
| | alpha CDR3a | |
| 52 | PD22_KRASMUT | AQTEISVVEKEDVTLDCVYETRDTTYYLFWYKQPPSGELVFLIR |
| | WT_1 TCR | RNSFDEQNEISGRYSWNFQKSTSSFNFTITASQVVDSAVYFCAL |
| | alpha variable | SE |
| | region | |
| 53 | PD22_KRASMUT | DVKVTQSSRYLVKRTGEKVFLECVQDMDHENMFWYRQDPGLGLR |
| | WT_1 TCR beta | LIYFSYDVKMKEKGDIPEGYSVSREKKERFSLILESASTNQTSM |
| | variable region | YLCASSL |
| 54 | PD22_KRASMUT | MLTASLLRAVIASICVVSSMAQKVTQAQTEISVVEKEDVTLDCV |
| | WT_1 TCR | YETRDTTYYLFWYKQPPSGELVFLIRRNSFDEQNEISGRYSWNF |
| | alpha chain | QKSTSSFNFTITASQVVDSAVYFCALSEALAGGSYIPTEGRGTS |
| | | LIVHPYIQNPDPAVYQLRDSKSSDKSVCLFTDFDSQTNVSQSKD |
| | | SDVYITDKTVLDMRSMDFKSNSAVAWSNKSDFACANAFNNSIIP |
| | | EDTFFPSPESSCDVKLVEKSFETDTNLNFQNLSVIGFRILLLKV |
| | | AGFNLLMTLRLWSS |
| 55 | PD22_KRASMU | MGIRLLCRVAFCFLAVGLVDVKVTQSSRYLVKRTGEKVFLECVQ |
| | TWT_1 TCR beta | DMDHENMFWYRQDPGLGLRLIYFSYDVKMKEKGDIPEGYSVSRE |
| | chain | KKERFSLILESASTNQTSMYLCASSLGPGQHNSPLHFGNGTRLT |
| | | VTEDLNKVFPPEVAVFEPSEAEISHTQKATLVCLATGFFPDHVE |
| | | LSWWVNGKEVHSGVSTDPQPLKEQPALNDSRYCLSSRLRVSATF |
| | | WQNPRNHFRCQVQFYGLSENDEWTQDRAKPVTQIVSAEAWGRAD |
| | | CGFTSVSYQQGVLSATILYEILLGKATLYAVLVSALVLMAMVKR |
| | | KDF |
| 56 | PD45_KRASMUT | SNHLY |
| | WT_4 TCR beta | |
| | CDR1b | |
| 57 | PD45_KRASMUT | FYNNEI |
| | WT_4 TCR beta | |
| | CDR2b | |
| 58 | PD45_KRASMUT | CASSAGSYEQYF |
| | WT_4 TCR beta | |
| | CDR3b | |
| 59 | PD45_KRASMUT | DSAIYN |
| | WT_4 TCR | |
| | alpha CDR1a | |
| 60 | PD45_KRASMUT | IQSSQRE |
| | WT_4 TCR | |
| | alpha CDR2a | |
| 61 | PD45_KRASMUT | CAVNSWSGAGSYQLTF |
| | WT_4 TCR | |
| | alpha CDR3a | |
| 62 | PD45_KRASMUT | KQEVTQIPAALSVPEGENLVLNCSFTDSAIYNLQWFRQDPGKGL |
| | WT_4 TCR | TSLLLIQSSQREQTSGRLNASLDKSSGRSTLYIAASQPGDSATY |
| | alpha variable | LCAV |
| | region | |
| 63 | PD45_KRASMUT | EPEVTQTPSHQVTQMGQEVILRCVPISNHLYFYWYRQILGQKVE |
| | WT_4 TCR beta | FLVSFYNNEISEKSEIFDDQFSVERPDGSNFTLKIRSTKLEDSA |
| | variable region | MYFCASSA |
| 64 | PD45_KRASMUT | METLLGLLILWLQLQWVSSKQEVTQIPAALSVPEGENLVLNCSF |
| | WT_4 TCR | TDSAIYNLQWFRQDPGKGLTSLLLIQSSQREQTSGRLNASLDKS |
| | alpha chain | SGRSTLYIAASQPGDSATYLCAVNSWSGAGSYQLTFGKGTKLSV |
| | | IPNIQNPDPAVYQLRDSKSSDKSVCLFTDFDSQTNVSQSKDSDV |
| | | YITDKTVLDMRSMDFKSNSAVAWSNKSDFACANAFNNSIIPEDT |
| | | FFPSPESSCDVKLVEKSFETDTNLNFQNLSVIGFRILLLKVAGF |
| | | NLLMTLRLWSS |
| 65 | PD45_KRASMUT | MDTWLVCWAIFSLLKAGLTEPEVTQTPSHQVTQMGQEVILRCVP |
| | WT_4 TCR beta | ISNHLYFYWYRQILGQKVEFLVSFYNNEISEKSEIFDDQFSVER |
| | chain | PDGSNFTLKIRSTKLEDSAMYFCASSAGSYEQYFGPGTRLTVTE |
| | | DLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDS |
| | | RG |
| 66 | EE208_KRAS_1 | MGHDK |
| | TCR beta CDR1b | |
| 67 | EE208_KRAS_1 | SYGVNS |
| | TCR beta CDR2b | |
| 68 | EE208_KRAS_1 | CASSESLYNEQFF |
| | TCR beta CDR3b | |
| 69 | EE208_KRAS_1 | ATGYPS |
| | TCR alpha CDR1a | |
| 70 | EE208_KRAS_1 | ATKADDK |
| | TCR alpha CDR2a | |
| 71 | EE208_KRAS_1 | CALSDGAGGFKTIF |
| | TCR alpha CDR3a | |
| 72 | EE208_KRAS_1 | GNSVTQMEGPVTLSEEAFLTINCTYTATGYPSLFWYVQYPGEGL |
| | TCR alpha | QLLLKATKADDKGSNKGFEATYRKETTSFHLEKGSVQVSDSAVY |
| | variable region | FCALS |
| 73 | EE208_KRAS_1 | EADIYQTPRYLVIGTGKKITLECSQTMGHDKMYWYQQDPGMELH |
| | TCR beta variable | LIHYSYGVNSTEKGDLSSESTVSRIRTEHFPLTLESARPSHTSQ |
| | region | YLCASSE |
| 74 | EE208_KRAS_1 | MNYSPGLVSLILLLLGRTRGNSVTQMEGPVTLSEEAFLTINCTY |
| | TCR alpha chain | TATGYPSLFWYVQYPGEGLQLLLKATKADDKGSNKGFEATYRKE |
| | | TTSFHLEKGSVQVSDSAVYFCALSDGAGGFKTIFGAGTRLFVKA |
| | | NIQNPDPAVYQLRDSKSSDKSVCLFTDFDSQTNVSQSKDSDVYI |
| | | TDKTVLDMRSMDFKSNSAVAWSNKSDFACANAFNNSIIPEDTFF |
| | | PSPESSCDVKLVEKSFETDINLNFQNLSVIGFRILLLKVAGENL |
| | | LMTLRLWSS |
| 75 | EE208_KRAS_1 | MTIRLLCYMGFYFLGAGLMEADIYQTPRYLVIGTGKKITLECSQ |
| | TCR beta chain | TMGHDKMYWYQQDPGMELHLIHYSYGVNSTEKGDLSSESTVSRI |
| | | RTEHFPLTLESARPSHTSQYLCASSESLYNEQFFGPGTRLTVLE |
| | | DLKNVEPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDS |
| | | RG |
| 76 | OX92_KRAS_36 | SGHVS |
| | TCR beta CDR1b | |
| 77 | OX92_KRAS_36 | FQNEAQ |
| | TCR beta CDR2b | |
| 78 | OX92_KRAS_36 | CASSLTGAGEQYF |
| | TCR beta CDR3b | |
| 79 | OX92_KRAS_36 | DSSSTY |
| | TCR alpha CDR1a | |
| 80 | OX92_KRAS_36 | IFSNMDM |
| | TCR alpha CDR2a | |
| 81 | OX92_KRAS_36 | CAERDPGNNRKLIW |
| | TCR alpha CDR3a | |
| 82 | OX92_KRAS_36 | GEDVEQSLFLSVREGDSSVINCTYTDSSSTYLYWYKQEPGAGLQ |
| | TCR alpha | LLTYIFSNMDMKQDQRLTVLLNKKDKHLSLRIADTQTGDSAIYF |
| | variable region | CAE |
| 83 | OX92_KRAS_36 | GAGVSQSPRYKVAKRGQDVALRCDPISGHVSLFWYQQALGQGPE |
| | TCR beta variable | FLTYFQNEAQLDKSGLPSDRFFAERPEGSVSTLKIQRTQQEDSA |
| | region | VYLCASSL |
| 84 | OX92_KRAS_36 | MKTFAGFSFLFLWLQLDCMSRGEDVEQSLFLSVREGDSSVINCT |
| | TCR alpha chain | YTDSSSTYLYWYKQEPGAGLQLLTYIFSNMDMKQDQRLTVLINK |
| | | KDKHLSLRIADTQTGDSAIYFCAERDPGNNRKLIWGLGTSLAVN |
| | | PNIQNPDPAVYQLRDSKSSDKSVCLFTDFDSQTNVSQSKDSDVY |
| | | ITDKTVLDMRSMDFKSNSAVAWSNKSDFACANAFNNSIIPEDTF |
| | | FPSPESSCDVKLVEKSFETDTNLNFQNLSVIGFRILLLKVAGFN |
| | | LLMTLRLWSS |
| 85 | OX92_KRAS_36 | MGTRLLCWVVLGFLGTDHTGAGVSQSPRYKVAKRGQDVALRCDP |
| | TCR beta chain | ISGHVSLFWYQQALGQGPEFLTYFQNEAQLDKSGLPSDRFFAER |
| | | PEGSVSTLKIQRTQQEDSAVYLCASSLTGAGEQYFGPGTRLTVT |
| | | EDLKNVFPPEVAVFEPSEAEISHTQKATLVCLATGFYPDHVELS |
| | | WWVNGKEVHSGVSTDPQPLKEQPALNDSRYCLSSRLRVSATFWQ |
| | | NPRNHFRCQVQFYGLSENDEWTQDRAKPVTQIVSAEAWGRADCG |
| | | FTSESYQQGVLSATILYEILLGKATLYAVLVSALVLMAMVKRKD |
| | | SRG |
| 86 | EE231_KRAS_52 | MDHEN |
| | TCR beta CDR1b | |
| 87 | EE231_KRAS_52 | SYDVKM |
| | TCR beta CDR2b | |
| 88 | EE231_KRAS_52 | CASSFGPGQHNSPLHF |
| | TCR beta CDR3b | |
| 89 | EE231_KRAS_52 | RDTTYY |
| | TCR alpha CDR1a | |
| 90 | EE231_KRAS_52 | RNSFDEQN |
| | TCR alpha CDR2a | |
| 91 | EE231_KRAS_52 | CALSEARTGGSYIPTF |
| | TCR alpha CDR3a | |
| 92 | EE231_KRAS_52 | AQKVTQAQTEISVVEKEDVTLDCVYETRDTTYYLFWYKQPPSGE |
| | TCR alpha | LVFLIRRNSFDEQNEISGRYSWNFQKSTSSENFTITASQVVDSA |
| | variable region | VYFCALSE |
| 93 | EE231_KRAS_52 | DVKVTQSSRYLVKRTGEKVFLECVQDMDHENMFWYRQDPGLGLR |
| | TCR beta variable | LIYFSYDVKMKEKGDIPEGYSVSREKKERFSLILESASTNQTSM |
| | region | YLCASSF |
| 94 | EE231_KRAS_52 | MLTASLLRAVIASICVVSSMAQKVTQAQTEISVVEKEDVTLDCV |
| | TCR alpha chain | YETRDTTYYLFWYKQPPSGELVFLIRRNSFDEQNEISGRYSWNF |
| | | QKSTSSFNFTITASQVVDSAVYFCALSEARTGGSYIPTFGRGTS |
| | | LIVHPYIQNPDPAVYQLRDSKSSDKSVCLFTDFDSQTNVSQSKD |
| | | SDVYITDKTVLDMRSMDFKSNSAVAWSNKSDFACANAFNNSIIP |
| | | EDTFFPSPESSCDVKLVEKSFETDTNLNFQNLSVIGFRILLLKV |
| | | AGFNLLMTLRLWSS |
| 95 | EE231_KRAS_52 | MGIRLLCRVAFCFLAVGLVDVKVTQSSRYLVKRTGEKVFLECVQ |
| | TCR beta chain | DMDHENMFWYRQDPGLGLRLIYFSYDVKMKEKGDIPEGYSVSRE |
| | | KKERFSLILESASTNQTSMYLCASSFGPGQHNSPLHEGNGTRLT |
| | | VTEDLNKVFPPEVAVFEPSEAEISHTQKATLVCLATGFFPDHVE |
| | | LSWWVNGKEVHSGVSTDPQPLKEQPALNDSRYCLSSRLRVSATF |
| | | WQNPRNHFRCQVQFYGLSENDEWTQDRAKPVTQIVSAEAWGRAD |
| | | CGFTSVSYQQGVLSATILYEILLGKATLYAVLVSALVLMAMVKR |
| | | KDF |
| 96 | sgTRAC | AGAGTCTCTCAGCTGGTACA |
| 97 | sgTRBC | GGAGAATGACGAGTGGACCC |
| 98 | sgRAB11A | GGTAGTCGTACTCGTCGTCG |
| 99 | sgCD4 | GGCAGGTCTTCTTCTCACTG |
| 100 | sgTNFRSF9 | GTTGAGGACCAGCAACAGAG |
| 101 | sgAAVS1 | GGGACCACCTTATATTCCCA |
| 102 | 10-MER | VVVGAGGVGK |
| | WILDTYPE | |
| | KRAS PEPTIDE | |
| 103 | 10-MER KRAS- | VVVGAVGVGK |
| | P.G12V PEPTIDE | |
| 104 | 9-MER | VVGAGGVGK |
| | WILDTYPE | |
| | KRAS PEPTIDE | |

What is claimed is:

1. A recombinant T cell receptor (TCR) that binds to a Kirsten rat sarcoma viral oncogene homolog (KRAS) G12D neoantigen comprising:
a TCR-alpha chain comprising a TCR-alpha variable region; and
a TCR-beta chain comprising a TCR-beta variable region;
wherein the TCR-alpha variable region comprises the following:
a CDR1 comprising the amino acid sequence SEQ ID NO: 7,
a CDR2 comprising the amino acid sequence SEQ ID NO: 8, and
a CDR3 comprising amino the acid sequence SEQ ID NO: 9; and
wherein the TCR-beta variable region comprises the following:
a CDR1 comprising the amino acid sequence SEQ ID NO: 3,
a CDR2 comprising the amino acid sequence SEQ ID NO: 4, and
a CDR3 comprising the amino acid sequence SEQ ID NO: 6.

2. The recombinant TCR of claim 1, wherein the TCR-alpha chain variable region comprises at least 95%, at least 96%, at least 97%, at least 98%, at least 99%, or 100% sequence identity to the amino acid sequence set forth in SEQ ID NO: 10 and the TCR-beta chain variable region comprises at least 95%, 96%, 97%, 98%, 99%, or 100% sequence identity to the amino acid sequence set forth in SEQ ID NO: 11.

3. The recombinant TCR of claim 1 or 2, wherein the TCR-alpha chain comprises at least 95%, at least 96%, at least 97%, at least 98%, at least 99%, or 100% sequence identity to the amino acid sequence set forth in SEQ ID NO: 12.

4. The recombinant TCR of any one of claims 1-3, wherein the TCR-beta chain comprises at least 95%, at least 96%, at least 97%, at least 98%, at least 99%, or 100% sequence identity to the amino acid sequence set forth in SEQ ID NO: 13, SEQ ID NO: 14, or 15.

5. The recombinant TCR of any one of claims 1-4, wherein the TCR binds the KRAS G12D neoantigen in a subject who is HLA-A*11:01 positive.

6. The recombinant TCR of any one of claims 1-5, wherein the TCR is HLA 11:01 restricted.

7. The recombinant TCR of any one of claims 1-6, wherein the TCR activates a T cell upon binding to the KRAS G12D neoantigen.

8. The recombinant TCR of any one of claims 1-7, wherein the neoantigen comprises SEQ ID NO: 1 or SEQ ID NO: 2.

9. An engineered cell that expresses the recombinant TCR of any one of claims 1-8.

10. The engineered cell of claim 9 wherein the cell is an iPSC-derived T cell, a patient-derived autologous T cell, a donor-derived T cell, or an iPSC cell, optionally wherein the patient-derived autologous T cell, donor-derived T cell, or iPSC-derived T cell is a CD8+ T cell.

11. The engineered cell of claim 9 or 10 wherein the cell is an iPSC-derived T cell.

12. The engineered cell of claim 9 or 10 wherein the cell is a patient-derived autologous T cell.

13. The engineered cell of any one of claims 9-12, wherein the T cell, which is optionally an iPSC-derived T cell, or iPSC cell comprises at least one nucleic acid sequence comprising at least one heterologous gene inserted into one or both of:
a. an endogenous T cell receptor alpha subunit constant gene (TRAC), and
b. an endogenous T cell receptor beta subunit constant gene (TRBC),
wherein the at least one heterologous gene comprises at least one of:
a. a variable region of a heterologous human TCR-a chain gene, and
b. a variable region of a heterologous human TCR-β chain gene.

14. The engineered cell of any one of claims 9-13, wherein the at least one nucleic acid sequence does not comprise a viral vector.

15. The engineered cell of any one of claims 9-14, wherein the T cell, which is optionally an iPSC-derived T cell, or iPSC cell comprises at least one nucleic acid sequence comprising at least one heterologous gene non-virally inserted into one or both of:
a. an endogenous T cell receptor alpha subunit constant gene (TRAC), and
b. an endogenous T cell receptor beta subunit constant gene (TRBC),
wherein the at least one heterologous gene comprises at least one of:
c. a variable region of a heterologous human TCR-a chain gene, and
d. a variable region of a heterologous human TCR-β chain gene.

16. The engineered cell of any one of claims 9-15, wherein the nucleic acid comprises a heterologous TCR-alpha subunit chain and a heterologous TCR-beta subunit chain.

17. The engineered cell of any one of claims 9-16, wherein the nucleic acid is inserted into the endogenous TRAC and the endogenous TRBC is deleted.

18. The engineered cell of any one of claims 9-17, wherein the nucleic acid comprises the variable region and constant region of a heterologous human TCR-β chain gene and the variable region of a heterologous human TCR-a chain gene.

19. The engineered cell of any one of claims 9-18, wherein the nucleic acid comprises, from N-terminus to C-terminus:
a. a first self-cleaving peptide sequence;
b. the variable region and constant region of a heterologous human TCR-β chain gene;
c. a second self-cleaving peptide sequence;
d. the variable region of a heterologous human TCR-a chain gene; and
e. a portion of the N-terminus of the endogenous TRAC.

20. The engineered cell of any one of claims 9-19, wherein the at least one heterologous gene replaces a placeholder TCR variable region.

21. A pharmaceutical composition comprising the engineered T cells of any one of claims 9-20.

22. A method for treating cancer in a subject in need thereof, comprising administering to the subject a therapeutically effective amount of the engineered T cell of any one of claims 9-20 or the pharmaceutical composition of claim 21.

23. The method of claim 22, wherein the subject is HLA-A*11:01 positive.

24. The method of claim 22 or 23, wherein the subject received prior therapy for treating the cancer.

25. The method of any one of claims 22-24, wherein the cancer is locally advanced, unresectable, metastatic, refractory, or recurrent cancer.

26. The method of any one of claims 22-25, wherein engineered T cells are administered at a dose of ≥7.5×10⁸ cells and ≤4.5×10¹⁰ cells.

27. The method of any one of claims 22-26, wherein the engineered T cells are administered via intravenous infusion.

28. The method of any one of claims 22-27 wherein the cancer is selected from the group consisting of: pancreatic cancer (e.g., pancreatic ductal adenocarcinoma (PDAC)), colorectal cancer (CRC), lung cancer (e.g., non-small cell lung cancer).

29. The method of any one of claims 22-28, further comprising administering an anti-PD-L1 antibody.

30. The method of claim 29, wherein the anti-PD-L1 antibody is atezolizumab.

31. The method of any one of claims 22-30, wherein the method further comprises administering to the subject a lymphodepleting chemotherapy regimen prior to administration of the engineered T cells.

32. The method of claim 31, wherein the lymphodepleting chemotherapy regimen comprises fludarabine and cyclophosphamide.

33. A recombinant T cell receptor (TCR) that binds to a KRAS G12D neoantigen, comprising a TCR-alpha chain variable region and a TCR-beta chain variable region, wherein
the TCR-alpha chain variable region comprises:
a. a CDR1 sequence comprising an amino acid sequence set forth in SEQ ID NOs: 19, 29, 39, 49, 59, 69, 79, or 89; and
b. a CDR2 sequence comprising an amino acid sequence set forth in SEQ ID NOs: 20, 30, 40, 50, 60, 70, 80, or 90; and
c. a CDR3 sequence comprising an amino acid sequence set forth in SEQ ID NOs: 21, 31, 41, 51, 61, 71, 81, or 91; and
the TCR-beta chain variable region comprises:
d. a CDR1 sequence comprising an amino acid sequence set forth in SEQ ID NOs: 16, 26, 36, 46, 56, 66, 76, or 86; and
e. a CDR2 sequence comprising an amino acid sequence set forth in SEQ ID NOs: 17, 27, 37, 47, 57, 67, 77, or 87; and
f. a CDR3 sequence comprising an amino acid sequence set forth in SEQ ID NOs: 18, 28, 38, 48, 58, 68, 78, or 88.

34. The recombinant TCR of claim 33, wherein the TCR comprises:
a. a TCR-alpha chain variable region comprising a CDR1, a CDR2, and a CDR3 comprising amino acid sequences SEQ ID NOs: 19, 20, and 21, respectively, and a TCR-beta chain variable region comprising a CDR1, a CDR2, and a CDR3 comprising amino acid sequences SEQ ID NOs: 16, 17, and 18, respectively; or
b. a TCR-alpha chain variable region comprising a CDR1, a CDR2, and a CDR3 comprising amino acid sequences SEQ ID NOs: 29, 30, and 31, respectively, and a TCR-beta chain variable region comprising a CDR1, a CDR2, and a CDR3 comprising amino acid sequences SEQ ID NOs: 26, 27, and 28, respectively; or
c. a TCR-alpha chain variable region comprising a CDR1, a CDR2, and a CDR3 comprising amino acid sequences SEQ ID NOs: 39, 40, and 41, respectively, and a TCR-beta chain variable region comprising a CDR1, a CDR2, and a CDR3 comprising amino acid sequences SEQ ID NOs: 36, 37, and 38, respectively; or
d. a TCR-alpha chain variable region comprising a CDR1, a CDR2, and a CDR3 comprising amino acid sequences SEQ ID NOs: 49, 50, and 51, respectively, and a TCR-beta chain variable region comprising a CDR1, a CDR2, and a CDR3 comprising amino acid sequences SEQ ID NOs: 46, 47, and 48, respectively; or
e. a TCR-alpha chain variable region comprising a CDR1, a CDR2, and a CDR3 comprising amino acid sequences SEQ ID NOs: 59, 60, and 61, respectively, and a TCR-beta chain variable region comprising a CDR1, a CDR2, and a CDR3 comprising amino acid sequences SEQ ID NOs: 56, 57, and 58, respectively; or
f. a TCR-alpha chain variable region comprising a CDR1, a CDR2, and a CDR3 comprising amino acid sequences SEQ ID NOs: 69, 70, and 71, respectively, and a TCR-beta chain variable region comprising a CDR1, a CDR2, and a CDR3 comprising amino acid sequences SEQ ID NOs: 66, 67, and 68, respectively; or
g. a TCR-alpha chain variable region comprising a CDR1, a CDR2, and a CDR3 comprising amino acid sequences SEQ ID NOs: 79, 80, and 81, respectively, and a TCR-beta chain variable region comprising a CDR1, a CDR2, and a CDR3 comprising amino acid sequences SEQ ID NOs: 76, 77, and 78, respectively; or
h. a TCR-alpha chain variable region comprising a CDR1, a CDR2, and a CDR3 comprising amino acid sequences SEQ ID NOs: 89, 90, and 91, respectively, and a TCR-beta chain variable region comprising a CDR1, a CDR2, and a CDR3 comprising amino acid sequences SEQ ID NOs: 86, 87, and 88, respectively.

35. The recombinant TCR of claim 33 or 34, wherein the TCR-alpha chain variable region comprises at least 95%, at least 96%, at least 97%, at least 98%, at least 99%, or 100% sequence identity to the amino acid sequence set forth in SEQ ID NO: 22, 32, 42, 52, 62, 72, 82, or 92.

36. The recombinant TCR of any one of claims 33-35, wherein the TCR-beta chain variable region comprises at least 95%, at least 96%, at least 97%, at least 98%, at least 99%, or 100% sequence identity to the amino acid sequence set forth in SEQ ID NO: 23, 33, 43, 53, 63, 73, 83, or 93.

37. The recombinant TCR of any one of claims 33-36, comprising:
a. a TCR-alpha chain variable region and a TCR-beta chain variable region having at least 95%, at least 96%, at least 97%, at least 98%, at least 99%, or 100% sequence identity to SEQ ID NO: 22 and SEQ ID: 23, respectively; or
b. a TCR-alpha chain variable region and a TCR-beta chain variable region having at least 95%, at least 96%, at least 97%, at least 98%, at least 99%, or 100% sequence identity to SEQ ID NO: 32 and SEQ ID: 33, respectively; or
c. a TCR-alpha chain variable region and a TCR-beta chain variable region having at least 95%, at least 96%, at least 97%, at least 98%, at least 99%, or 100% sequence identity to SEQ ID NO: 42 and SEQ ID: 43, respectively; or
d. a TCR-alpha chain variable region and a TCR-beta chain variable region having at least 95%, at least 96%, at least 97%, at least 98%, at least 99%, or 100% sequence identity to SEQ ID NO: 52 and SEQ ID: 53, respectively; or
e. a TCR-alpha chain variable region and a TCR-beta chain variable region having at least 95%, at least 96%, at least 97%, at least 98%, at least 99%, or 100% sequence identity to SEQ ID NO: 62 and SEQ ID: 63, respectively; or
f. a TCR-alpha chain variable region and a TCR-beta chain variable region having at least 95%, at least 96%, at least 97%, at least 98%, at least 99%, or 100% sequence identity to SEQ ID NO: 72 and SEQ ID: 73, respectively; or
g. a TCR-alpha chain variable region and a TCR-beta chain variable region having at least 95%, at least 96%, at least 97%, at least 98%, at least 99%, or 100% sequence identity to SEQ ID NO: 82 and SEQ ID: 83, respectively; or
h. a TCR-alpha chain variable region and a TCR-beta chain variable region having at least 95%, at least 96%, at least 97%, at least 98%, at least 99%, or 100% sequence identity to SEQ ID NO: 92 and SEQ ID: 93, respectively.

38. The recombinant TCR of any one of claims 33-37, comprising:
a. a TCR-alpha chain and a TCR-beta chain having at least 95%, at least 96%, at least 97%, at least 98%, at least 99%, or 100% sequence identity to SEQ ID NO: 24 and SEQ ID: 25, respectively; or
b. a TCR-alpha chain and a TCR-beta chain having at least 95%, at least 96%, at least 97%, at least 98%, at least 99%, or 100% sequence identity to SEQ ID NO: 34 and SEQ ID: 35, respectively; or
c. a TCR-alpha chain and a TCR-beta chain having at least 95%, at least 96%, at least 97%, at least 98%, at least 99%, or 100% sequence identity to SEQ ID NO: 44 and SEQ ID: 45, respectively; or
d. a TCR-alpha chain and a TCR-beta chain having at least 95%, at least 96%, at least 97%, at least 98%, at least 99%, or 100% sequence identity to SEQ ID NO: 54 and SEQ ID: 55, respectively; or
e. a TCR-alpha chain and a TCR-beta chain having at least 95%, at least 96%, at least 97%, at least 98%, at least 99%, or 100% sequence identity to SEQ ID NO: 64 and SEQ ID: 65, respectively; or
f. a TCR-alpha chain and a TCR-beta chain having at least 95%, at least 96%, at least 97%, at least 98%, at least 99%, or 100% sequence identity to SEQ ID NO: 74 and SEQ ID: 75, respectively; or
g. a TCR-alpha chain and a TCR-beta chain having at least 95%, at least 96%, at least 97%, at least 98%, at least 99%, or 100% sequence identity to SEQ ID NO: 84 and SEQ ID: 85, respectively; or
h. a TCR-alpha chain and a TCR-beta chain having at least 95%, at least 96%, at least 97%, at least 98%, at least 99%, or 100% sequence identity to SEQ ID NO: 94 and SEQ ID: 95, respectively.

39. The recombinant TCR of any one of claims 33-38, wherein the TCR binds the KRAS G12D neoantigen in a subject who is HLA-A*11:01 positive.

40. The recombinant TCR of any one of claims 33-39, wherein the TCR is HLA 11:01 restricted.

41. The recombinant TCR of any one of claims 33-40, wherein the TCR activates a T cell upon binding to the KRAS G12D neoantigen.

42. The recombinant TCR of any one of claims 33-41, wherein the neoantigen comprises SEQ ID NO: 1 or SEQ ID NO: 2.

43. An engineered cell that expresses the recombinant TCR of any one of claims 33-42.

44. The engineered cell of claim 43 wherein the cell is an iPSC-derived T cell, a patient-derived autologous T cell, a donor-derived T cell, or an iPSC cell, optionally wherein the patient-derived autologous T cell, donor-derived T cell, or iPSC-derived T cell is a CD8+ T cell.

45. The engineered cell of claim 43 or 44 wherein the cell is an iPSC-derived T cell.

46. The engineered cell of claim 43 or 44 wherein the cell is a patient-derived autologous T cell.

47. The engineered cell of any one of claims 43-46, wherein the T cell, which is optionally an iPSC-derived T cell, or iPSC cell comprises at least one nucleic acid sequence comprising at least one heterologous gene inserted into one or both of:
a. an endogenous T cell receptor alpha subunit constant gene (TRAC), and
b. an endogenous T cell receptor beta subunit constant gene (TRBC),
wherein the at least one heterologous gene comprises at least one of:
e. a variable region of a heterologous human TCR-a chain gene, and
f. a variable region of a heterologous human TCR-β chain gene.

48. The engineered cell of claim 43-47, wherein the at least one nucleic acid sequence does not comprise a viral vector.

49. The engineered cell of claim 43-48, wherein the T cell, which is optionally an iPSC-derived T cell, or iPSC cell comprises at least one nucleic acid sequence comprising at least one heterologous gene non-virally inserted into one or both of:
a. an endogenous T cell receptor alpha subunit constant gene (TRAC), and
b. an endogenous T cell receptor beta subunit constant gene (TRBC),
wherein the at least one heterologous gene comprises at least one of:
g. a variable region of a heterologous human TCR-a chain gene, and
h. a variable region of a heterologous human TCR-β chain gene.

50. The engineered cell of claim 43-49, wherein the nucleic acid comprises a heterologous TCR-alpha subunit chain and a heterologous TCR-beta subunit chain.

51. The engineered cell of claim 43-50, wherein the nucleic acid is inserted into the endogenous TRAC and the endogenous TRBC is deleted.

52. The engineered cell of claim 43-51, wherein the nucleic acid comprises the variable region and constant region of a heterologous human TCR-β chain gene and the variable region of a heterologous human TCR-a chain gene.

53. The engineered cell of claim 43-52, wherein the nucleic acid comprises, from N-terminus to C-terminus:
a. a first self-cleaving peptide sequence;
b. the variable region and constant region of a heterologous human TCR-β chain gene;
c. a second self-cleaving peptide sequence;
d. the variable region of a heterologous human TCR-a chain gene; and
e. a portion of the N-terminus of the endogenous TRAC.

54. The engineered cell of claim 43-53, wherein the at least one heterologous gene replaces a placeholder TCR variable region.

55. A pharmaceutical composition comprising the engineered T cells of any one of claims 43-54.

56. A method for treating cancer in a subject in need thereof, comprising administering to the subject a therapeutically effective amount of the engineered T cell of any one of claims 43-54 or the pharmaceutical composition of claim 55.

57. The method of claim 56, wherein the subject is HLA-A*11:01 positive.

58. The method of claim 56 or 57, wherein the subject received prior therapy for treating the cancer.

59. The method of any one of claims 56-58, wherein the cancer is locally advanced, unresectable, metastatic, refractory, or recurrent cancer.

60. The method of any one of claims 56-59, wherein engineered T cells are administered at a dose of ≥7.5×10⁸ cells and ≤4.5×10¹⁰ cells.

61. The method of any one of claims 56-60, wherein the engineered T cells are administered via intravenous infusion.

62. The method of any one of claims 56-61 wherein the cancer is selected from the group consisting of: pancreatic cancer (e.g., pancreatic ductal adenocarcinoma (PDAC)), colorectal cancer (CRC), lung cancer (e.g., non-small cell lung cancer).

63. The method of any one of claims 56-62, further comprising administering an anti-PD-L1 antibody.

64. The method of claim 62, wherein the anti-PD-L1 antibody is atezolizumab.

65. The method of any one of claims 56-64, wherein the method further comprises administering to the subject a lymphodepleting chemotherapy regimen prior to administration of the engineered T cells.

66. The method of claim 65, wherein the lymphodepleting chemotherapy regimen comprises fludarabine and cyclophosphamide.

67. An engineered cell that expresses a recombinant TCR comprising:
a TCR-alpha chain comprising a TCR-alpha variable region; and
a TCR-beta chain comprising a TCR-beta variable region;
wherein the TCR-alpha variable region comprises the following:
a. a CDR1 comprising the amino acid sequence SEQ ID NO: 7,
b. a CDR2 comprising the amino acid sequence SEQ ID NO: 8, and
c. a CDR3 comprising amino the acid sequence SEQ ID NO: 9; and
wherein the TCR-beta variable region comprises the following:
d. a CDR1 comprising the amino acid sequence SEQ ID NO: 3,
e. a CDR2 comprising the amino acid sequence SEQ ID NO: 4, and
f. a CDR3 comprising the amino acid sequence SEQ ID NO: 6; and
wherein the T cell, which is optionally an iPSC-derived T cell, or iPSC cell comprises at least one nucleic acid sequence comprising at least one heterologous gene inserted into one or both of:
a. an endogenous T cell receptor alpha subunit constant gene (TRAC), and
b. an endogenous T cell receptor beta subunit constant gene (TRBC),
wherein the at least one heterologous gene comprises at least one of:
a. a variable region of a heterologous human TCR-a chain gene, and
b. a variable region of a heterologous human TCR-β chain gene; and
wherein intracellular delivery of the at least one heterologous gene comprises introducing the at least one heterologous gene using a viral vector.

68. An engineered cell that expresses a recombinant TCR comprising:
a TCR-alpha chain comprising a TCR-alpha variable region; and
a TCR-beta chain comprising a TCR-beta variable region;
wherein the TCR-alpha chain variable region comprises at least 95%, at least 96%, at least 97%, at least 98%, at least 99%, or 100% sequence identity to the amino acid sequence set forth in SEQ ID NO: 10 and the TCR-beta chain variable region comprises at least 95%, 96%, 97%, 98%, 99%, or 100% sequence identity to the amino acid sequence set forth in SEQ ID NO: 11; and
wherein the T cell, which is optionally an iPSC-derived T cell, or iPSC cell comprises at least one nucleic acid sequence comprising at least one heterologous gene inserted into one or both of:
a. an endogenous T cell receptor alpha subunit constant gene (TRAC), and
b. an endogenous T cell receptor beta subunit constant gene (TRBC),
wherein the at least one heterologous gene comprises at least one of:
a. a variable region of a heterologous human TCR-a chain gene, and
b. a variable region of a heterologous human TCR-β chain gene; and
wherein intracellular delivery of the at least one heterologous gene comprises introducing the at least one heterologous gene using a viral vector.

69. An engineered cell that expresses a recombinant TCR comprising:
a TCR-alpha chain comprising a TCR-alpha variable region; and
a TCR-beta chain comprising a TCR-beta variable region;
wherein the TCR-alpha chain comprises at least 95%, at least 96%, at least 97%, at least 98%, at least 99%, or 100% sequence identity to the amino acid sequence set forth in SEQ ID NO: 12; and
wherein the TCR-beta chain comprises at least 95%, at least 96%, at least 97%, at least 98%, at least 99%, or 100% sequence identity to the amino acid sequence set forth in SEQ ID NO:
13, SEQ ID NO: 14, or 15; and
wherein the T cell, which is optionally an iPSC-derived T cell, or iPSC cell comprises at least one nucleic acid sequence comprising at least one heterologous gene inserted into one or both of:
a. an endogenous T cell receptor alpha subunit constant gene (TRAC), and
b. an endogenous T cell receptor beta subunit constant gene (TRBC),
wherein the at least one heterologous gene comprises at least one of:
a. a variable region of a heterologous human TCR-a chain gene, and
b. a variable region of a heterologous human TCR-β chain gene; and
wherein intracellular delivery of the at least one heterologous gene comprises introducing the at least one heterologous gene using a viral vector.

70. A method of preparing the engineered cell of any one of claims 67-69 comprising
a. providing a cell, and
b. introducing at least one heterologous gene in a viral vector into the cell,
wherein the viral vector comprises a nucleic acid sequence encoding at least the TCR alpha subunit and/or the TCR beta subunit,
wherein optionally the TCR alpha and/or TCR beta subunits are inserted into the host cell at the TCR alpha locus and/or TCR beta locus, respectively, and/or at a locus outside of the endogenous TCR alpha locus and/or TCR beta locus.
